SEQUENCE LISTING (29 sequences)

SEQ ID NO: 1 - protein, 4 aa, Artificial Sequence
  Synthetic peptide
KRFR

SEQ ID NO: 2 - protein, 4 aa, Artificial Sequence
  Synthetic peptide
ITAM

SEQ ID NO: 3 - protein, 4 aa, Artificial Sequence
  Synthetic peptide
KRFH

SEQ ID NO: 4 - protein, 61 aa, Homo sapiens
LPIMFDVLIHDPSHFLNYSTINYKWSFGDNTGLFVSTNHTVNHTYVLNGTFSLNLTVKAAA

SEQ ID NO: 5 - protein, 64 aa, Mus musculus
LRDLPIVFDVLIHDPSHFLNDSAISYKWNFGDNTGLFVSNNHTLNHTYVLNGTFNLNLTVQTAV

SEQ ID NO: 6 - RNA, 19 nt, Artificial Sequence
  Synthetic primer
aacuugucugaugagaucu

SEQ ID NO: 7 - RNA, 19 nt, Artificial Sequence
  Synthetic primer
agaucucaucagacaaguu

SEQ ID NO: 8 - RNA, 19 nt, Artificial Sequence
  Synthetic primer
gcguacaagccaauaggaa

SEQ ID NO: 9 - RNA, 19 nt, Artificial Sequence
  Synthetic primer
uuccuauuggcuuguacgc

SEQ ID NO: 10 - DNA, 24 bp, Artificial Sequence
  Synthetic primer
ccctcccgcaaattgtggctgtaa

SEQ ID NO: 11 - DNA, 22 bp, Artificial Sequence
  Synthetic primer
ccccgtgcggatgagatgtgac

SEQ ID NO: 12 - DNA, 17 bp, Artificial Sequence
  Synthetic primer
ccggggcgcagggagaa

SEQ ID NO: 13 - DNA, 19 bp, Artificial Sequence
  Synthetic primer
tttgggggaagcagcacta

SEQ ID NO: 14 - DNA, 24 bp, Artificial Sequence
  Synthetic primer
cttggacacagaggccccgacacc

SEQ ID NO: 15 - DNA, 22 bp, Artificial Sequence
  Synthetic primer
cgcccaccaccccacccacgat

SEQ ID NO: 16 - DNA, 21 bp, Artificial Sequence
  Synthetic primer
cctccccgacgacgaagatgc

SEQ ID NO: 17 - DNA, 19 bp, Artificial Sequence
  Synthetic primer
aacgcccgccacccacaac

SEQ ID NO: 18 - DNA, 22 bp, Artificial Sequence
  Synthetic primer
gtggagcttcggggataatact

SEQ ID NO: 19 - DNA, 22 bp, Artificial Sequence
  Synthetic primer
ctactcagctccagggggttgt

SEQ ID NO: 20 - DNA, 24 bp, Artificial Sequence
  Synthetic primer
gttcaccataagccaagtcacacc

SEQ ID NO: 21 - DNA, 24 bp, Artificial Sequence
  Synthetic primer
ttgccccagcttatatttccacag

SEQ ID NO: 22 - DNA, 21 bp, Artificial Sequence
  Synthetic primer
gacctggccctgcactctcct

SEQ ID NO: 23 - DNA, 24 bp, Artificial Sequence
  Synthetic primer
aaaaacaaccccgaactaactgct

SEQ ID NO: 24 - DNA, 24 bp, Artificial Sequence
  Synthetic primer
atgccctgtgaaatactgtgctaa

SEQ ID NO: 25 - DNA, 20 bp, Artificial Sequence
  Synthetic primer
tgcctggtgcttgcttctgt

SEQ ID NO: 26 - DNA, 18 bp, Artificial Sequence
  Synthetic primer
gggcccggcgcaatgaca

SEQ ID NO: 27 - DNA, 18 bp, Artificial Sequence
  Synthetic primer
gcgggcgggggatgaggt

SEQ ID NO: 28 - DNA, 24 bp, Artificial Sequence
  Synthetic primer
ctccgctccagcaccttctacaac

SEQ ID NO: 29 - DNA, 23 bp, Artificial Sequence
  Synthetic primer
gcaccgtcagggctcttccagtc

(19) United States
(12) Patent Application Publication
(10) Pub. No.: US 2009/0297479 A1
(43) Pub. Date: Dec. 3, 2009

(54) DC-HIL CONJUGATES FOR TREATMENT OF T-CELL DISORDERS

(76) Inventors: Kiyoshi Ariizumi, Dallas, TX (US); Ponciano D. Cruz, Dallas, TX (US)

Correspondence Address: FULBRIGHT & JAWORSKI L.L.P., 600 CONGRESS AVE., SUITE 2400, AUSTIN, TX, 78701, US

(21) Appl. No.: 12/411,898

(22) Filed: Mar. 26, 2009

Related U.S. Application Data
(60) Provisional application No. 61/040,524, filed on Mar. 28, 2008.

(51) Int. Cl. A61K 38/21; A61K 39/395; C12N 9/00; C07K 19/00

(52) U.S. Cl. 424/85.7; 424/173.1; 424/178.1; 424/183.1; 435/183; 530/391.7

(57) ABSTRACT

The present invention relates to the use of DC-HIL and fragments and variants thereof to selectively target toxins to activated T-cells expressing a unique form of syndecan-4 that is not found on other cells. Thus, the toxin is delivered only to activated T-cells, and not to other syndecan-4 expressing cells. Such toxin-DC-HIL conjugates are useful in the treatment of T-cell inflammatory disorders such as dermatitis, autoimmune disease, and graft rejection, as well as T-cell lymphomas.

This application claims benefit of priority to U.S. Provisional Application Ser. No. 61/040,524, file Mar. 28, 2008, the entire contents of which are hereby incorporated by reference.

The government owns rights in the present invention pursuant to grant number RO1-A164927-01 from the National Institutes of Health.

### BACKGROUND OF THE INVENTION

1. Field of the Invention

The present invention relates to fields of immunology, oncology and medicine. More particularly, the present invention relates to the use of toxin-conjugates that preferentially target activated T-cells. In particular, such conjugates may find use in the treatment of diseases such as T-cell cancers, inflammation and autoimmune diseases.

2. Description of Related Art

T-cell activation is dependent on signals delivered by antigen-presenting cells (APCs) to the antigen (Ag)-specific T-cell receptor (TCR) and accessory receptors on T-cells (Chambers and Allison, 1997). The principal stimulatory accessory signal is transmitted by B7-1 (CD80) or B7-2 (CD86) on APCs to the CD28 receptor on T-cells (Acuto and Michel, 2003). Interestingly, engagement of the same B7-1 or B7-2 ligand to CTLA-4 (CD152) on T-cells markedly attenuates T-cell responses (Walunas et al., 1994; Krummel and Allison, 1995). The importance of CTLA-4 as an inhibitory regulator of T-cell activation is illustrated by death of CTLA-4-deficient mice within 4 weeks of birth because of massive lymphocytic infiltration destroying critical organs (Tivol et al., 1995).

More recently, other inhibitory regulators of T-cell activation were identified, including PD-L1 (B7-H1) and PD-L2 (B7-DC) on APCs and PD-1 on T-cells (Okazaki et al., 2002), BTLA on B cells and T helper 1 (Th1) effector cells and its ligand (herpes virus entry mediator) on T-cells (Sedy et al., 2005; Watanabe et al., 2003), and Tim-3 on APCs and Th1 effector cells and Tim-3 ligand on CD4 T-cells (Monney et al., 2002; Sabatos et al., 2003; Sanchez-Fueyo et al., 2003). The T-cell ligands possess a single immunoglobulin (Ig)-like variable (IgV) domain, and the APC receptors contain both IgV and Ig constant (IgC) domains.₁₂ Interactions between ligand-receptor pairs are mediated predominantly by residues of Ig-like domains (Carreno and Collins, 2002). Because of their structural and functional similarities to B7 molecules, these ligands/receptors are considered members of the B7 receptor superfamily (Carreno and Collins, 2002).

Ligation of PD-1 on T-cells leads to inhibited T-cell responses that can be rescued by exogenous IL-2 or CD28 costimulation (Latchman et al., 2001; Tseng et al., 2001; Freeman et al., 2000), although one report showed that binding of PD-L1 (B7-H1) to PD-1 stimulated T-cell proliferation and IL-10 secretion (Dong et al., 1999; Dong and Chen, 2003; Subudhi et al., 2004). PD-1 deficiency leads to exaggerated autoimmunity since PD-1 knockout mice develop splenomegaly, increased numbers of B and myeloid cells, increased serum IgG and IgA, and a lupus erythematosus-like disease with age (Nishimura et al., 1999; Nishimura et al., 1998). These mice are also markedly susceptible to Ag-induced experimental autoimmune encephalomyelitis (EAE) (Nishimura et al., 1999; Nishimura et al., 1998). BTLA knockout mice do not exhibit developmental Tor B-cell defects, but their lymphocytes have heightened responses to anti-CD3 antibody (Ab) and to anti-IgM Ab (Watanabe et al., 2003) these mice are also prone to developing EAE (Watanabe et al., 2003). In the case of the Tim-3 pathway, its blockade by monoclonal Ab (mAb), Fc-fused soluble receptor, or gene disruption leads to exacerbated Th1-mediated autoimmune diabetes mellitus in nonobese diabetic (NOD) mice (Sabatos et al., 2003; Sanchez-Fueyo et al., 2003).

T-cell expression of PD-1, BTLA or Tim-3 resembles CTLA-4 in that it is not constitutive, but is induced by activation (Liang and Sha, 2002). Moreover, the costimulation delivered by each appears to be mediated through the TCR (Carreno and Collins, 2002). By contrast, expression of PD-1, BTLA, Tim-3, or their ligands differ from CTLA-4 in that it is not restricted to T-cells, but is expressed more widely to include B cells and APCs. Indeed, some of these ligands (PD-L1 and PD-L2) are also expressed in nonlymphoid tissues (Carreno and Collins, 2002). Such broad expression profiles suggest that these molecules can modulate immune responses in secondary lymphoid organs and peripheral tissues (Okazaki et al., 2002), consistent with the observation that IFN-γ can induce expression of PD-1 ligands on non-lymphoid cells (Latchman et al., 2001).

Previously, the inventors identified DC-HIL as a highly glycosylated type I transmembrane protein of 125 and 95 kDa containing an extracellular Ig-like domain (Shikano et al., 2001). They also showed that DC-HIL is expressed constitutively at high levels on the surface of all dendritic cell subsets, including plasmacytoid dendritic cells and Langerhans cells and at lower levels on macrophages (Shikano et al., 2001), and that its expression can be induced in non-lymphoid cells (keratinocytes) following IFN-γ treatment. In human, DC-HIL is expressed constitutively at high levels by CD14⁺ monocytes and dendritic cells (but not by other leukocytes). They have also shown that DC-HIL is a negative regulator of T-cell activation (Chung et al., 2007a; Chung et al., 2007b) through binding to syndecan-4 on activated T-cells, indicating that interaction of DC-HIL with syndecan-4 attenuates T-cell activation triggered by anti-CD3 Ab or by APCs in a manner resembling the inhibitory function of PD-L1/PD-L2.

### SUMMARY OF THE INVENTION

Thus, in accordance with the present invention, there is provided a method of reducing T-cell induced inflammation in a subject comprising administering to said subject a conjugate comprising (a) DC-HIL or a syndecan-4-binding fragment thereof; and (b) a toxin. The T-cell induced inflammation may be host-versus-graft disease, psoriasis, atopic dermatitis, contact hypersensitivity, autoimmune disease, or skin graft rejection. The subject may be a human, a mouse, a rat, a dog or a cat. The syndecan-4-binding fragment of DC-HIL comprising the DC-HIL Ig-like domain. The toxin may be saporin, ricin, botulinum toxin or diptheria toxin. Administration may comprise intravenous, intra-arterial, topical, intralesional, subcutaneous, intraperitoneal, intradermal, or intranasal administration. The method may further comprise administering to said subject a second anti-inflammatory treatment, such as an immunosuppressant, a steroid or an NSAID. The method may further comprise a second administration of said conjugate.

In another embodiment, there is provided a method of inhibiting a syndecan-4-positive T-lymphoma or T-leukemia cell in a subject comprising administering to said subject a conjugate comprising (a) DC-HIL or a syndecan-4-binding fragment thereof; and (b) a toxin. Inhibiting comprises reducing the viability or proliferation of said cell. The subject may be a human, a mouse, a rat, a dog or a cat. The syndecan-4-binding fragment of DC-HIL may comprise the DC-HIL Ig-like domain. The toxin may be saporin, ricin, botulinum toxin or diptheria toxin. Administration may comprise intravenous, intra-arterial, intra-lymphatic, intralesional, subcutaneous, intraperitoneal, intradermal or intranasal administration. The method may further comprise administering to said subject a second anti-lymphoma or -leukemia treatment, such as chemotherapy, radiotherapy, IFNα and/or anti-CD20 antibody. The method may further comprise a second administration of said conjugate.

In yet another embodiment, there is provided a conjugate comprising (a) DC-HIL or a syndecan-4-binding fragment thereof; and (b) a toxin. The syndecan-4-binding fragment of DC-HIL may comprise the DC-HIL Ig-like domain. The toxin may be saporin, ricin, or diptheria toxin. The conjugate may be disposed in a pharmaceutically acceptable buffer, carrier or diluent.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

These, and other, embodiments of the invention will be better appreciated and understood when considered in conjunction with the following description and the accompanying drawings. It should be understood, however, that the following description, while indicating various embodiments of the invention and numerous specific details thereof, is given by way of illustration and not of limitation. Many substitutions, modifications, additions and/or rearrangements may be made within the scope of the invention without departing from the spirit thereof, and the invention includes all such substitutions, modifications, additions and/or rearrangements.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-B—DC-HIL-Fc binds to activated (but not resting) T-cells. (FIG. 1A) Purified splenic CD4⁺ T-cells were cultured with concanavalin A (10 μg/mL) for 3 days. After blocking the binding activity of Fc receptors on cells with a Fc blocker, treated T-cells were stained with DC-HIL-Fc/FITC-anti-human IgG Ab (open histograms), or corresponding control Abs (gray histograms). Binding of DC-HIL-Fc to T-cells was analyzed by FACS. (FIG. 1B) Purified CD3⁺ T-cells were treated with immobilized anti-CD3 Ab (1 μg/mL) for 3 days and then stained with FITC-labeled anti-CD4 or anti-CD8 Ab and with DC-HIL-Fc or hIgG/PE-anti-human IgG. Numbers in each quadrant represent percentages of the total cell population. Data shown are representative of 3 independent experiments.

FIGS. 2A-J—Immobilized DC-HIL-Fc inhibits T-cell activation triggered by anti-CD3 Ab. CD4⁺ (FIGS. 2A-B) or CD8⁺ (FIGS. 2C-D) T-cells (2×10⁵ each) purified from BALB/c spleens were cultured for 48 hours in microculture wells precoated with increasing doses of anti-CD3 Ab and with a constant dose (10 μg/mL) of DC-HIL-Fc (), control hIgG (◯), or neither (none; Δ). After pulsing with ³H-thymidine, cells and culture supernatant were harvested. ³H-thymidine incorporation into cells (FIGS. 2A,C) and IL-2 production (FIGS. 2B,D) were determined, and values (cpm or ng/mL) plotted at a logarithmic scale, respectively. (FIG. 2E) Titration of inhibitory function of DC-HIL-Fc. CD4⁺ T-cells were cultured for 48 hours in microculture wells precoated with a constant dose (0.3 μg/mL) of anti-CD3 Ab and increasing doses of DC-HIL-Fc or control hIgG. (FIG. 2F) CD28 costimulation rescues DC-HIL-Fc-induced inhibition of CD4⁺ T-cell activation. Purified CD4⁺ T-cells were cultured in wells precoated with anti-CD3Ab (0.3 μg/mL), hIgG or DC-HIL-Fc (5 μg/mL), and anti-CD28 mAb (increasing doses). (FIGS. 2G-H). Previously activated T-cells were prepared from Tac-TgN(DO11.10)-Rag2tm1 mice and reactivated by immobilized anti-CD3Ab (varying doses) and DC-HIL-Fc or hIgG (constant dose). (FIGS. 2I-J) Inhibitory function of DC-HIL-Fc was titrated against reactivation of previously activated T-cells by anti-CD3 Ab (1 μg/mL). Proliferation (FIGS. 2G,I) and IL-2 production (FIGS. 2H and 2J) were measured. Results are expressed as mean values±SDs. Data shown are representative of 6 (FIGS. 2A-B), 3 (FIGS. 2G-J), and 2 (FIGS. 2C-F) experiments, respectively.

FIGS. 3A-B—Cell-cycle analyses of CD4⁺ T-cells treated with DC-HIL-Fc. (FIG. 3A) T-cells (6×10⁶) were labeled with CFSE (1 μM) and then cultured in microwells precoated with anti-CD3Ab (0.3 μg/mL) plus hIgG or DC-HIL-Fc (5 μg/mL). At the indicated time points, cells were harvested and analyzed by FACS for fluorescence intensity. The frequency (%) of divided cells is shown in histograms. (FIG. 3B) T-cells from 48-hour culture similarly treated were analyzed for incorporation of BrdU (using FITC-anti-BrdU Ab) and total DNA content (stained with 7-AAD) by FACS; data shown as dot plots of BrdU versus 7-AAD. Data shown are representative of 3 (FIG. 3A) and 2 (FIG. 3B) independent experiments.

FIGS. 4A-H—Soluble DC-HIL-Fc enhances responses of CD4⁺ T-cells by APCs. Effects of soluble DC-HIL-Fc on T-cell activation were examined in MLR (FIGS. 4A-B), anti-CD3 response (FIG. 4C), or in OVA-specific antigen presentation (FIGS. 4D-E). MLR: C57BL/6 spleen cells (5×10⁴) were γ-irradiated and mixed with CD4⁺ T-cells (2×10⁵) purified from BALB/c mouse spleens. (FIG. 2A) Increasing doses of hIgG or DC-HIL-Fc were added to the MLR culture and incubated for 2 days prior to ³H-thymidine pulsing. Proliferative response of T-cells was assayed by incorporation of ³H-thymidine. (FIG. 4B) MLR was incubated in the absence/presence of hIgG or DC-HIL-Fc (20 μg/mL) for 1, 2, or 3 days before pulsing. (FIG. 4C) Soluble (Sol) DC-HIL-Fc does not inhibit T-cell activation triggered by immobilized (Im) anti-CD3 Ab. CD4⁺ T-cells were cultured in microwells precoated with anti-CD3 Ab (0.3 μg/mL) and 5 μg/mL of DC-HIL-Fc. In some wells, soluble hIgG or DC-HIL-Fc in increasing doses was added to culture in wells coated with the same amount of anti-CD3 Ab. T-cell activation was expressed as proliferative capacity. (FIGS. 4D-E) OVA-specific response: CD4⁺ T-cells purified from the spleens of BALB/cTac-TgN(DO11.10)-Rag2tm1 mice were cocultured without (No) or with BM-DCs (from BALB/c mice) previously pulsed with OVApeptide. T-cell activation was assayed by IL-2 production (FIG. 4D) and by FACS for frequency of CD69⁺/CD4⁺T-cells (FIG. 4E). Control staining was performed with FITC-rat IgG (rIgG) and PE-hamster IgG (haIgG). (FIG. 4F) siRNA-mediated knockdown of DC-HIL. At 1 day after transfection of DCs with control (Ctrl; shuffled) siRNA or DC-HIL-targeted siRNA, cells were harvested and assayed by immunoblotting for protein expression of DC-HIL or β-actin. (FIG. 4G) Increasing numbers of transfected DCs were pulsed with OVA peptide and cocultured with a constant number of OVA-specific CD4⁺ T-cells. Activation was measured by IL-2 production. (FIG. 4H) At 2 days after coculturing, frequency of CD69⁺ in the CD4⁺ T-cells was determined by FACS. *Statistical significance (P<0.001) compared with T-cell responses treated with hIgG control. Data shown are representative of at least 3 independent experiments.

FIGS. 5A-C—Soluble DC-HIL-Fc enhances elicitation of Ox-induced contact hypersensitivity in mice. Sensitization of BALB/c mice (n=5) with Ox for CH (FIGS. 5A-B): on day 0, mice were painted with 2% Ox on abdominal skin (Senst). On day 6, CH was elicited in sensitized mice by painting 1% Ox or solvent control to right and left ears, respectively (Challenge). CH was assessed daily through day 9 or 12 by measuring ear thickness (OE). Mice were injected intraperitoneally with PBS, hIgG, or DC-HIL-Fc (10 mg/kg each) on days −1, 1, and 3 (before and after sensitization) (FIG. 5A) or on days 5, 7, and 9 (before and after challenge) (FIG. 5B). Daily change in ear thickness was plotted for each panel during sensitization (FIG. 5A) or elicitation (FIG. 5B). *P<0.003; **P<0.05 compared with ear thickness of mice treated with hIgG. (FIGS. 5C) Ear skin was excised from mice treated without (None) or with Ox and Fc protein (2 days after elicitation) and examined histologically (10×/10 objective lens). Data shown (FIGS. 5A-B) are representative of 4 independent experiments.

FIGS. 6A-D—Ox/DC-HIL-Fc-treated LN cells display hyperactivation phenotypes. In an independent experiment, draining LN (DLN) cells prepared from BALB/c mice treated similarly (as in FIGS. 5A-C) were examined. (FIG. 6A) DLN cells were counted. (FIG. 6B) Spontaneous activation was measured by 3H-thymidine incorporation of DLN cells (4×10⁵/well) cultured for 3 days without stimuli. (FIGS. 6C-D) frequency of leukocytes: DLN cells were stained with FITC-Ab against CD4, CD8, or B220 alone (FIGS. 6C) or doubly stained with PE-anti-CD69 (FIG. 6D), and then analyzed by FACS. CD69 expression (FIG. 6D) is shown in LN cells stained positively with the surface marker Ab. Results (FIGS. 6A and 6B) are shown as mean values±SDs; *P<0.001 compared with LN responses treated with hIgG.

FIGS. 7A-D—Mutant analyses of DC-HIL-Fc function. (FIG. 7A) Protein structures of DC-HIL-Fc wild-type (WT) and mutants are represented schematically. Extracelluar domains (ECDs) of mutants, RAA (replacement of RGD sequence with RAA; OE) and deletion mutants lacking PRR (amino acid [aa] 301-334) and PKD230-355 were linked to a Fc portion of hIgG and produced in COS-1 cells. (FIG. 7B) After purifying mutant DC-HIL-Fc proteins, a small aliquot (2 μg/lane) was run on SDS-PAGE and then stained with Coomassie Blue to visualize protein bands. (FIG. 7C) T-cell activation. Highly purified DC-HIL-Fc WT or mutants (5 μg/mL each) and anti-CD3 Ab (increasing doses) were coated on microwells for culture with CD4⁺ T-cells for 2 days and pulsed with ³H-thymidine for 20 hours. Results are shown as mean values±SDs. (FIG. 7D) Binding of DC-HIL mutants to T-cells. Activated CD4⁺ T-cells were incubated with WT and mutants of DC-HIL (10 μg/mL) and analyzed for binding by FACS. Histograms of T-cells stained with hIgG (filled) and a mutant (open) are overlaid. Data control. Data shown are representative of 3 independent experiments.

FIGS. 8A-F—Among HSPG, SD-4 is likely a ligand of DC-HIL. (FIG. 8A) Heparin blocks binding of DC-HIL to T-cells. Activated CD4⁺ T-cells were incubated with control Ig (shaded histograms) or DC-HIL-Fc (unshaded histograms) (10 μg/ml) in the absence (None) or presence of heparin at indicated concentrations. After labeling cell-bound DC-HIL-Fc with PE-anti-human IgG Ab, fluorescence intensity on cells was examined by FACS. (FIG. 8B) Heparin blocks DC-HIL's inhibitory function. Splenic CD4⁺ T-cells were treated with immobilized anti-CD3 Ab (0.3 μg/ml) and control Ig (open circles) or DC-HIL-Fc (5 μg/ml, closed circles) pretreated with varying doses of heparin (in triplicate). T-cell activation was measured by ³H-thymidine incorporation. (FIG. 8C) mRNA expression of SDs by T-cells. Total RNA was isolated from resting or activated CD4⁺ or CD8⁺ T-cells and mRNA expression of SDs and β-actin determined by RT-PCR. (FIG. 8D) Surface expression of SD on CD4⁺ T-cells. Resting (day 0) or activated CD4⁺ T-cells (day 3 after activation by immobilized anti-CD3 Ab) were determined by FACS for surface expression of SD-1, SD-3 and SD-4. (FIG. 8E) Surface expression of SD-4 by CD8⁺ T-cells. Resting (day 0) and activated CD8⁺ T-cells (similarly treated as CD4⁺ T-cells) were examined for surface expression of SD-4 by FACS. (FIG. 8F) Protein expression of SD-1 and SD-4. Whole cell protein extracts (7×10⁵ cells equivalent/lane) prepared from resting and activated CD4⁺ T-cells were subjected to immunoblotting using Ab to SD-1 or SD-4. Arrows indicate molecular weights of SD-1 and SD-4. All data are representative of at least 2 independent experiments.

FIGS. 9A-E—DC-HIL binds SD-4 on T-cells. (FIG. 9A) Immunoprecipitation (IP) of SD-4 with DC-HIL-Fc. Protein extract prepared from activated T-cells was incubated without ((just extract) or with control Ig or DC-HIL-Fc (5 μg/ml) and immunoprecipitants examined by immunoblotting for expression of SD-4. (FIG. 9B) Pretreatment of DC-HIL-Fc with SD-4-Fc blocks binding of DC-HIL to T-cells. DC-HIL-Fc was pretreated with SD4-Fc or control Ig at varying concentrations prior to binding to activated CD4⁺ T-cells. Binding was determined by FACS. (FIG. 9C) Pretreatment of T-cells with anti-SD-4 Ab blocks binding of DC-HIL to T-cells. Activated CD4⁺ T-cells were pretreated with anti-SD-4 Ab or isotypic IgG prior to binding assays with DC-HIL-Fc. (FIG. 9D) Transgene expression of V5-SD4 by DO11.10 T-cells. DO11.10 T-cells infected with a lentiviral vector encoding both Emerald GFP and V5-SD4 were stained with anti-V5 Ab, anti-SD-4 Ab, or mouse (or rat) IgG and surface labeling measured by FACS. Frequency (%) of GFP⁺/V5⁺ or GFP⁺/SD-4⁺ cells is shown in the dot-blots. (FIG. 9E) Binding of DC-HIL-Fc to DO11.10 T-cells. DO11.10 parental cells or those with expression of V5-SD4 were incubated with control Ig (shaded histograms) or DC-HIL-Fc (open histograms), followed by FACS. Binding assay was also performed in the presence of heparin (2 μg/ml). All data shown are representative of 3 independent experiments.

FIGS. 10A-E—SD-4 inhibits anti-CD3 responses. Function of SD-4 was analyzed using DO11.10 T-cell lines (FIGS. 10A-B), splenic CD4⁺ T-cells (FIG. 10C), MLR (FIG. 10D), and OVA-T-cell activation by DC (FIG. 1E). (FIGS. 10A-B) DO11.10 T-cells expressing V5-SD4 (V5-SD4-DO, closed circles) or GFP (GFP-DO, open circles) were treated with immobilized anti-CD3 Ab (1 μg/ml) and DC-HIL-Fc (FIG. 10A) or anti-V5 Ab (FIG. 10B) at varying doses. T-cell activation was measured by IL-2 production and is expressed as %, relative to IL-2 produced by anti-CD3 alone (9.5±0.7 ng/ml by GFP-DO and 8.8±0.7 ng/ml by V5-SD4-DO set as 100%). (FIG. 10C) CD4⁺ T-cells were treated with a constant dose of control IgG or anti-SD-4 Ab (10 μg/ml) and increasing doses of anti-CD3 Ab (all Ab are biotinylated) and then cross-linked by streptavidin-coated magnetic beads. Proliferative capacity of treated T-cells was measured by ³H-thymidine incorporation. (FIG. 10D) BALB/c CD4⁺ T-cells (responders) were mixed with spleen cells (stimulators) of C57BL/6 mouse in the presence of control Ig, SD4-Fc, or DC-HIL-Fc at different doses. T-cell activation was determined by ³H-thymidine incorporation. (FIG. 10E) CD4⁺ T-cells were co-cultured with OVA peptide-pulsed BM-DC in the presence of SD4-Fc or control Ig at different doses. T-cell alone (no DC) and PBS-added cultures served as controls. T-cell activation was evaluated by IL-2 production. Data shown are representative of at least 3 independent experiments.

FIGS. 11A-C—T-cells knocked-down for SD-4 respond more strongly to DC. (FIG. 11A) Efficacy of siRNA. Whole cell extracts were prepared from COS-1 cells untransfected or co-transfected with a SD-4 gene and SD-4-targeted SC-siRNA or Sf-siRNA oligonucleotide (control) and determined by immunoblotting for protein expression of SD-4 (left) or β-actin (right). (FIG. 11B) Knock-down of SD-4 expression in T-cells. Splenic CD4⁺ T-cells freshly isolated from DO11.10 transgenic mice were untreated, pulsed alone or pulsed with SC-siRNA or Sf-siRNA using Amaxa system and cultured with immobilized anti-CD3 Ab. After culturing for 2 d, surface expression of SD-4 or PD-1 was measured by FACS. (FIG. 11C) OVA-antigen presentation. CD4⁺ T-cells transfected with siRNA were co-cultured for 1 or 2 d with BM-DC (from wild-type mice) pulsed with the OVA peptide. IL-2 production was measured. Data shown are representative of 3 independent experiments.

FIGS. 12A-H—Infusion of anti-SD-4 or SD4-Fc in mice enhances elicitation of contact hypersensitivity. Sensitization of BALB/c mice (n=5) with oxazolone (Ox) for contact hypersensitivity (CH) (FIGS. 12A-C): on day 0, mice were sensitized by painting 2% Ox on abdominal skin. On day 6, CH was elicited in sensitized mice by painting 1% Ox or solvent control to right or left ears, respectively (Challenge). Ear thickness was measured daily from day 1 through day 5 following challenge. Mice were also given i.p. injection of PBS, control IgG, or anti-SD-4 Ab 3 h prior to the sensitization (FIG. 12A) or challenge (FIG. 12B). Some panels of mice were i.p. injected with PBS, control Ig, or SD4-Fc before challenge (FIG. 12C). Daily change in ear thickness (×10⁻³ inch) after challenge was plotted for each panel. Statistical significance is denoted by *(p<0.001) as compared to ear thickness treated with control Ig. (FIG. 12D) Histological examination of ear skin. Mice treated with Ox and PBS, control IgG, or anti-SD-4 Ab were sacrificed 2 d after challenge; ear skin biopsies were stained with H & E and examined histologically (10× magnification). In an independent experiment, LN cells prepared from BALB/c mice treated similarly were examined: (FIG. 12E) LN cells counted, and (FIG. 12F) their spontaneous activation measured by ³H-thymidine incorporation of LN cells cultured for 3 d without stimuli. (FIGS. 12G and H) Frequency of leukocytes: LN cells were stained with FITC-Ab against CD4, CD8, or B220 alone (FIG. 12G) or doubly-stained with PE-anti-CD69 (FIG. 12H), and then analyzed by FACS. CD69 expression (FIG. 12H) is shown in LN cells stained positively with the surface marker Ab. Data shown are representative of 3 independent experiments (FIGS. 12A-D) and 2 experiments (FIGS. 12E-H).

FIG. 13—Kinetics of SD-4 expression in LN T-cells during CH. LN cells of untreated BALB/c mice (Ct) or mice at different days after challenge with Ox (day 0, just prior to challenge) were doubly-stained with PE-anti-SD-4 Ab (or PE-anti-PD-1 Ab) and FITC-anti-CD4 or CD8, followed by FACS for expression of SD-4 and PD-1 in CD4⁺ and CD8⁺ T-cells. Each value is shown as frequency (%) of SD-4⁺ or PD-1⁺ cells in the total number of CD4⁺ or CD8⁺ T-cells, with standard errors derived from 3 independent experiments.

FIGS. 14A-E—Effects of DC-HIL-SAP on T-cells in vitro. Conjugation with SAP (saporin): DC-HIL-Fc or control Ig was biotynylated (one protein molecule has 1-2 biotin molecules) and then coupled with streptavidin-SAP (Advanced Targeting System) at a molecular ratio of 5:1. (FIG. 14A) Binding to T-cells. Splenic T-cells from BALB/c mice were left untreated (resting) or activated by anti-CD3 Ab (2 μg/ml) for 3 d. These cells were incubated with 5 μg/ml DC-HIL-SAP (open histograms) or Ig-SAP (shaded), followed by fluorescence labeling with PE-anti-human IgG Ab. Binding was measured by FACS. (FIGS. 14B-C) Effects on T-cell proliferation. Splenic CD4⁺ (FIG. 14B) or CD8⁺ T-cells (FIG. 14C) (2×10⁵ cells/well, in triplicate) were activated with immobilized anti-CD3 Ab (2 μg/ml) for 2 d, and then different doses of DC-HIL-SAP or Ig-SAP (shown as SAP concentration) were added to the culture and incubated for one more day. Next day, ³H-thymidine was pulsed for 20 h prior to harvest. Relative cpm to the control (0 nM or PBS) is expressed as %. (FIG. 14D) Killing of SD-4+T-cells. Similarly treated T-cells (just prior to harvest) were stained with anti-SD-4 Ab and % of SD-4⁺ T-cells is shown. (FIG. 14E) Internalization: activated T-cells were incubated with DC-HIL-SAP (5 μg/ml) and fluorescently labeled with FITC-anti-human IgG Fab fragment on ice. After washing, cells were split into two batches: one batch was kept on ice and the other was incubated at 37° C. for 30 min. Cells were then fixed and fluorescence was analyzed using confocal microscopy.

FIGS. 15A-C—Effects of DC-HIL-SAP on contact hypersensitivity(CH). CH was induced in BALB/c mice (6-10 wks; female): On day 0, mice (n=4) were sensitized by painting 2% oxazolone (Ox) on shaved abdominal skin. On day 6, CH was elicited in sensitized mice by painting 1% Ox or solvent control to right or left ears, respectively (Challenge). Ear thickness was measured daily from day 1 through day 3 or 4 following challenge. (FIG. 15A) Mice were also injected i.v. (200 μl/mouse) with PBS, Ig-SAP, or DC-HIL-SAP at saporin concentration of 20 (left panel) or 40 nM (right) 3 h prior to the challenge. (FIG. 15B) The 40 nM injections were also performed 3 h prior to sensitization. Daily change in ear thickness (10⁻³ in) was plotted for each panel. Statistical significance denoted by * represents p<0.1 in comparison to ear thickness treated with PBS. All other data were p<0.001 in comparison to ear thickness of mice treated with Ig-SAP. Data shown are representative of 2 (FIGS. 15B-C) and 3 (FIG. 15A) independent experiments.

FIGS. 16A-C—Ox-unresposiveness lasts 3 weeks. CH was induced in BALB/c mice as described previously. (FIG. 16A) Mice were also injected i.v. (200 μl/mouse) with PBS, Ig-SAP, or DC-HIL-SAP at 20 (left panel) or 40 nM (right) 3 h prior to the challenge on day 6. Mice were then challenged with Ox additional 3 times with 1 week-apart. Ear thickness after the second challenge (FIG. 16A), the third (FIG. 16B) and the fourth (FIG. 16C) was measured.

FIG. 17—Infusion of DC-HIL-SAP shifts from Th1 to Th2-like response. BALB/c mice were sensitized and challenged as described previously. Mice were also given intravenous injection of 40 nM Ig-SAP or DC-HIL-SAP 3 h prior to challenge. One day post-challenge, draining lymph node cells (1×10⁶ cells/well) were cultured with immobilized anti-CD3 Ab (1 μg/ml) for 3 days. Production of IFN-γ (Th1 cytokine) and IL-4 (Th2 cytokine) was measured by ELIZA.

FIGS. 18—Antigen-specific unresponsiveness induced by infusion of DC-SAP. BLAB/c mice were sensitized, challenged with OX, and received intravenous injection of PBS, Ig-SAP or DC-HIL-SAP (40 nM). Mice injected with DC-HIL blocked elicitation of CH response by nearly 90%. One week after the challenge (all mice had no ear swelling), mice were sensitized (day 12 after Ox sensitization) and challenged (day 18) with DNCB contact allergen (1%). Ear thickness was measured daily.

FIGS. 19A-L—Binding of DC-HIL to activated T cells leads to attenuated anti-CD3 response. (FIG. 19A) At different time points after stimulation with PMA/ionomycin, CD4⁺ T cells were harvested and incubated with DC-HIL-Fc (open histograms) or control Ig (filled in gray) and PE-anti-mouse IgG Ab. Activated CD4+T cells were also immunostained with anti-CD69 mAb or control IgG. Binding of DC-HIL-Fc and expression of CD69 were examined by flow cytometry. (FIGS. 19B-I) Purified CD4⁺ (FIGS. 19B-E) or CD8⁺ T cells (FIGS. 19F-I) were cultured in 96-well plates (in triplicate) pre-coated with DC-HIL-Fc (closed circles) or control (Ctrl) Ig (open circles) (each 10 μg/ml) plus anti-CD3 Ab at increasing doses. After culturing for 2 d, T cell proliferation was measured by ³H-thymidine (TdR) incorporation (FIGS. 19B and F) or production of IL-2 (FIGS. 19C and G), TNF-A (FIGS. 19D and H), or IFN-Y (FIGS. 19E and I) (mean±SD, n=3). (FIG. 19J) CD4⁺ T cell activation triggered by anti-CD3 Ab (0.3 μg/ml) was treated with increasing doses of DC-HIL-Fc or control Ig. (FIG. 19K) Anti-CD3 response (0.3 μg/ml) of T cells treated with DC-HIL-Fc or control Ig (5 μg/ml) was cultured with increasing doses of anti-CD28 Ab. T cells treated with anti-CD3 Ab (0.3 μg/ml)/DC-HIL or control Ig (each 5 μg/ml) were cultured with increasing doses of anti-CD28 Ab. Activation was measured by ³H-thymidine (mean±SD, n=3). (FIG. 19L) After culturing with anti-CD3 Ab (0.3 μg/ml) and DC-HIL-Fc or control Ig (5 μg/ml) for 2 d, cells were subjected to cell cycle analysis using BrdU incorporation (FITC-anti-BrdU) and 7-AAD (labeling total DNA). Dot-blot analysis of BrdU/7-AAD staining is shown. All data shown are representative of at least 3 separate experiments.

FIGS. 20A-H—SD-4 is the T cell ligand of DC-HIL. (FIG. 20A) mRNA expression of SD-1, -2, -3, -4, or GAPDH was examined by RT-PCR in total RNA isolated from resting CD4⁺ T cells or those activated with PHA or PMA/ionomycin. (FIG. 20B) Resting or PMA/ionomycin-activated CD4⁺ T cells were stained with Ab against SD-1, -2, -3, -4 (unshaded histograms) or control IgG (shaded), and surface expression examined by flow cytometry. (FIG. 20C) Whole cell extracts prepared from activated CD4⁺ T cells were immunoprecipitated with DC-HIL-Fc or control Ig and then immunoblotted with anti-SD-1, anti-SD-4, or control IgG. (FIG. 20D) Jurkat cells transfected with vector alone (Ctrl), SD-1 or SD-4 gene were examined by flow cytometry for surface expression of SD-1 or SD-4. These Jurkat transfectants were also treated with Con A and then examined by flow cytometry for binding of DC-HIL-Fc (unshaded hisotograms) or control Ig (shaded). (FIGS. 20E and F) Binding of DC-HIL-Fc to Con A-activated SD-4+Jurkat cells was performed in the presence of heparin at indicated concentrations. SD-4+Jurkat cells were also pretreated with anti-SD-4 Ab or control IgG (FIG. 20E) or without (None) or with heparinase prior to binding to DC-HIL (FIG. 20F). (FIGS. 20G and H) Phosphorylation of SD-4. Jurkat cells were transfected with SD-4-V5 gene and stimulated with Con A prior to incubation with immobilized DC-HIL-Fc. At varying time points after incubation, SD-4-V5 protein was immunoprecipitated with anti-V5 Ab, and serine (FIG. 20G) and tyrosine (FIG. 20H) phosphorylation assayed by immunoblotting with Ab to serine-phosphorylated SD-4 (p-Ser-SD-4) and to phosphorylated tyrosine (p-Tyr), respectively. In each precipitant, the amount of SD-4-V5 protein precipitated was examined by immunoblotting. All data shown are representative of at least 2 separate experiments.

FIGS. 21A-D—SD-4 acts as a negative regulator of anti-CD3 response. (FIG. 21A) Three Jurkat transfectants were stimulated with immobilized anti-CD3 Ab (varying doses) and DC-HIL-Fc or control Ig (a constant dose of 10 μg/ml) for 2 d. T cell activation was measured by IL-2 production (mean±SD, n=3). (FIG. 21B) Peripheral blood CD4⁺ T cells were cultured in 96-well plates precoated with anti-CD3 Ab at increasing doses and DC-HIL-Fc, anti-SD-1, anti-SD-4 Ab, or control Ig at each 10 μg/ml. (FIGS. 21C and D) Inhibitory function of anti-SD-4 Ab (10 μg/ml) was compared with Ab directed against other inhibitory receptors (PD-1 and CTLA-4) by titrating with increasing doses of anti-CD3 Ab (FIG. 21C) or by titrating a constant dose of anti-CD3 Ab (0.3 μg/ml) with increasing anti-inhibitory receptor Ab (FIG. 21D). All treated T cells (FIGS. 21B-D) were cultured for 3 d and activation assessed by ³H-thymidine incorporation (mean±SD, n=3). Effects of DC-HIL-Fc or anti-SD-4 Ab at all dose points were statistically significant (Student's t test p<0.05), compared to control Ig or other Ab. All data shown are representative of at least 2 independent experiments.

FIGS. 22A-F—Expression of DC-HIL by human leukocytes. (FIG. 22A) mRNA expression of DC-HIL or GAPDH was examined by RT-PCR of total RNA prepared from freshly isolated (resting) or activated CD4+, CD8+ T cells, CD19+ B cells, and CD14+ monocytes. (FIG. 22B) PBMC were immunostained with 3D5 anti-DC-HIL and anti-CD 14 mAb or isotype control IgG. Fluorescent labeling was examined by flow cytometry; quadro-analysis is shown. (FIG. 22C) Following stimulation with relevant cytokines or LPS for 2 d, PBMC were examined for surface expression of DC-HIL and CD14 by flow cytometry. Effects on surface expression of DC-HIL on CD14+ cells were assessed by fold differences calculated by mean fluorescence intensity on treated/untreated cells (None) (FIG. 22D) (mean±SD, n=3). * p<0.05 vs. None using Student's t test. (FIG. 22E) Expression on epidermal LC. Epidermal cell suspensions were fluorescently stained with 3D5 mAb (PE-labeling) and anti-CD1a Ab (FITC-labeling), and surface expression was analyzed by flow cytometry. (FIG. 22F) Immature and mature DC were doubly-stained with anti-HLA-DR and 3D5 mAb or Ab to either one of two activation markers (CD80 and CD86). All data shown are representative of 3 independent experiments.

FIGS. 23A-F—DC-HIL expression correlates inversely with allostimulatory capacity of CD14+ monocytes. (FIG. 23A) γ-irradiated PBMC (stimulators) were mixed with allogeneic CD4+ T cells (responders) at varying ratios and cultured for 6 d in the absence (None) or presence of DC-HIL-Fc or control Ig. Proliferation was measured by ³H-thymidine incorporation. T cell activation is expressed as cpm after deduction of background cpm (control culture in which y-irradiated responders and unirradiated stimulators were mixed) from experimental cpm (mean±SD, n=3). (FIG. 23B) CD 14+ cells were untreated or treated with TGF-β (10 ng/ml) for 2 d and then co-cultured with allogeneic CD4+ T cells at a ratio of 0.1:1 or 0.2:1 (CD 14+ cells:CD4+ T cells). T cell activation was assessed by IL-2 production (mean±SD, n=3). (FIG. 23C) TGF-β-treated or untreated CD14+ cells were co-cultured with CD4+ T cells at a ratio of 0.2:1 in the presence of DC-HIL-Fc or control Ig (mean±SD, n=3). (FIGS. 23D and E) CD 14+ cells were transfected with DC-HIL-targeted or control siRNA. 2 d post-transfection, protein and surface expression of DC-HIL or β-actin was examined by immunoblotting (FIG. 23D) and flow cytometry (FIG. 23E), respectively. (FIG. 23F) siRNA-transfected CD14+ cells were allowed to activate allogeneic CD4+ T cells in co-culture with varying ratios. 6 d after co-culturing, IL-2 production was measured (mean±SD, n=3). *p<0.05 vs. values treated with control Ig using Student's t test.

FIG. 24—DC-HIL inhibits cytokine production by activated T cells on a per cell basis. CD4+ or CD8+ T cells (2×10⁵ cells/well, in triplicate) purified from PBMCs were cultured for 3 d in microculture wells precoated with anti-CD3 Ab (0.3 μg/ml) and DC-HIL-Fc or control Ig (10 μg/ml). Cells were harvested from 3 wells, pooled, permeabilized, and then stained with PE-conjugated anti-IL-2, anti-IFN-γ, pooled, permeabilized, and then stained with PE-conjugated anti-IL-2, anti-IFN-γ, and anti-TNF-α. Numbers in quadrants indicate the percentage of cytokine-positive cells. Data shown are representative of 2 independent experiments.

FIGS. 25A-D—Human DC-HIL α and β isoforms possess heparin-binding activity. (FIG. 25A) mRNA expression of human DC-HIL was examined by RT-PCR of total RNA prepared from PBMCs. PCR products were size-fractionated through 0.8% agarose gel running. The upper and lower PCR bands correspond to α (full-length) and β isoforms (truncated form), respectively. (FIG. 25B) A full-length cDNA encoding the β isoform was cloned and sequenced, and deduced amino acid sequence (560 amino acids) was aligned with the α isoform (572 amino acids) consisting of leader sequence (open), extracellular (gray), transmembrane (black), and intracellular (open) domains. (FIG. 25C) Varying concentrations (μM) of mouse DC-HIL-Fc or human DC-HIL-Fc (α and β isoforms) were incubated in ELISA wells (triplicate) chemically coated with heparin (500 μg/ml). After washing, DC-HIL-Fc protein bound to heparin was assayed by ELISA. Specific binding is expressed as OD450 reading left after subtracting background by control mouse IgG from experimental OD₄₅₀. (FIG. 25D) Binding of human α isoform (0.9 μM) to heparin was inhibited by different doses (μg/ml) of CS (chondroitin sulfate), H (heparan sulfate), and heparin. Second set of experiments (for FIGS. 25C and 25D) showed similar results.

FIGS. 26A-C—Expression of SD-4 by CTCL lines. (FIG. 26A) Total RNA isolated from 3 CTCL or 2 adult T cell leukemia (ATCL) lines was examined by RT-PCR for mRNA expression of SD-4, other known co-inhibitors, CD148, or GAPDH. (FIG. 26B) These 5 cell lines and peripheral CD4⁺ T cells (CD4-T, day 3 after activation with anti-CD3 Ab) were also assayed by flowcytometry for surface expression of SD-4 and CD148. (FIG. 26C) These CTCL lines were stained with FITC-anti-SD-4 Ab or control IgG and surface expression analyzed by confocal microscopy.

FIGS. 27A-C—CD4⁺ T cells in PBMCs of CTCL patients express SD-4 at markedly upregulated levels as compared to those of normal donors. PBMCs isolated from CTCL patients with different stages of lympho-malignancy (see Table 2) or normal donors were examined by flowcytometry for surface expression of SD-4. (FIG. 27A) Dot-blots and quadro-analysis are shown using flowcytometry data of sample #7 fluorescently stained with control IgG (IgG) or with anti-SD-4 and anti-CD4 Ab. Frequency (%) of CD4⁺ SD-4⁺ cells is calculated. (FIG. 27B) Flow cytometry data of 11 patients and 3 normal donors are summarized and expressed as a graphical version. (FIG. 27C) Relationship between % of CD4+ SD-4⁺ and of CD4⁺ CD26− PBMCs from 11 patients tested is shown, with the degree (r) of liner relationship evaluated by the Pearson correlation.

FIG. 28A-D—DC-HIL binds to MJ and HUT-78 CTCL through particular types of heparin sulfate saccharide. (FIG. 28A) Three CTCL lines, two ATCL lines, or human normal CD4⁺ T cells (in vitro activated) were incubated with control Ig (open histograms) or DC-HIL-Fc (filled in gray) and stained with PE-anti-mouse IgG. Binding of DC-HIL was assayed by flowcytometry. (FIG. 28B) HUT-78 cells were left untreated (None) or treated with heparinase before incubation with DC-HIL-Fc. Binding of DC-HIL was assayed before. (FIG. 28C) Three CTCL lines or activated normal CD4⁺ T cells were stained with 3 different anti-heparan sulfate mAb (F58-10E4, Hepss-1, and F69-3G10) or control IgG (open histograms). Expression each epitope was assessed by flowcytometry. (FIG. 28D) HUT-78 cells were pretreated with the 3 anti-heparan sulfate mAb or control IgG (10 or 40 μg/ml) before DC-HIL binding assay. Mean fluorescence intensity is shown in the histograms.

FIGS. 29A-B—SD-4+HUT-78 cells respond to inhibitory function of DC-HIL by suppressing IL-2 secretion. HUT-78 cells (FIG. 29A) or HH (FIG. 29B) or were cultured with immobilized anti-CD3 Ab (increasing doses) plus 5 μg/ml DC-HIL-Fc (closed circles) or control Ig (open circles). Activation was measured by IL-2 production for the HUT-78 line (FIG. 29A) or by % frequency of CD69⁺ cells for the HH line (FIG. 29B) (mean±sd, n=3).

FIGS. 30A-B—Treatment with DC-HIL has no effect on proliferation of HUT-78 and HH cells triggered by anti-CD3 Ab. (FIG. 30A) HUT-78 or HH cells were cultured with immobilized anti-CD3 Ab (increasing doses) plus 5 μg/ml DC-HIL-Fc (closed circles) or control Ig (open circles) and then pulsed with ³H-thymdine (TdR) in the last 20 h of the culture period. Incorporation of ³H-htymidine (cpm) into cells is shown with mean±sd, n=3. (FIG. 30B) These CTCL lines were starved with serum-free media and stimulated by adding FCS to the media, and cultured for 18 h. Cells were harvested and subjected to cell cycle analysis using FITC-anti-BrdU and 7-AAD. Dot-blot analysis is shown with location of each cell cycle phase.

FIGS. 31A-B—Anti-CTCL activity of saporin-conjugated DC-HIL. HUT-78 (FIG. 31A) or HH cells (FIG. 31B) were cultured with varying concentrations (mM) of saporin-conjugated DC-HIL or control saporin for 2 days. Proliferation of treated cells was measured by incorporation of ³H-thimidine. Effect of saporin conjugates on proliferation is expressed as % relative to cpm taken by untreated cells.

### DESCRIPTION OF ILLUSTRATIVE EMBODIMENTS

The inventors have previously demonstrated that human DC-HIL on antigen presenting cells inhibited T-cell functions on activated T-cells, but not resting T-cells, via syndecan-4 (SD-4), a transmembrane (type I) heparan sulfate proteoglycan. Here, they now show that a DC-HIL conjugate of ribosome-inactivating toxin saporin (DC-HIL-SAP) bound to activated SD-4⁺ effector T-cells, but not to SD-4⁻ regulatory T-cells, nor did it bind to other SD-4⁺ cell types, including B-cells which express SD-4 constitutively at high level. After binding to SD-4 on activated T-cells, DC-HIL-SAP is internalized and exhibits cytotoxic activity.

In an oxazolone-sensitized mouse model, administration of DC-HIL-SAP suppressed ear-swelling to almost baseline level when compared to SAP alone. The DC-HIL-SAP recipient mice survived without visible adverse effects and displayed suppressed contact hypersensitivity responses to additional oxazolone challenges up to 6 weeks. Twenty percent of CD69⁺ (a T-cell activation marker) cells from draining lymph nodes (DLN) of oxazolone-challenged mice are also SD-4⁺. Infusion of DC-HIL-SAP suppressed T-cell activation as the number of CD69⁺ cells in DLN was reduced by 50%. There was also a 30% drop of SD-4⁺ cells. No difference in the numbers of CD4⁺, CD8⁺ or B220⁺ cells between DC-HIL-SAP and SAP treated DLN was observed. SD-4⁺ cells almost disappeared in DC-HIL-SAP-treated mouse skin where oxazolone was applied directly. The inventors further showed that DC-HIL binds to heparan sulfate saccharide on syndecan-4 on activated T-cells but not to syndecan-4 on other cell-types, which is likely to be produced only by activated T-cells.

The inventors also found that SD-4 is not expressed by regulatory T cells (T_{reg}) that can protect host against graft-versus-host disease caused by bone marrow transplantation. Treatment of T_{reg} in vitro with DC-HIL-SAP has null effect on their function. Injection of DC-HIL-SAP in mice has no deterious effect on T_{reg} in spleen and lymph nodes. These results indicate that injection of DC-HIL-SAP in mice deactivates function of effector T cells while preserving T_{reg} function. Finally, the inventors found that T cells of patients with cutaneous T cell lymphoma (mycosis fungoides and Sezary syndrome) express syndecan-4 on the cell surface and the expression level correlates positively with the stages of Sezary syndrome (the degree of peripheral blood involvement). Further characterization of SD-4 expression by T cells, SD-4 is not expressed by regulatory T cells (T_{reg}) that play a critical role in suppressing immune responses. In consistent with this absence, DC-HIL-SAP does not have deterious effect on T_{reg} in vitro and in mice. SD-4 is expressed at elevated levels by malignant T cells of patients with cutaneous T cell lymphoma (CTCL). In in vitro culture, DC-HIL-SAP kills a cell line HUT-78 derived from Sezary syndrome, an aggressive form of CTCL.

Based on these results, the inventors propose the use of DC-HIL-toxin conjugates for the treatment of various T-cell inflammatory diseases. In addition, examination of clinical pathologic specimen from cutaneous lymphoma patients revealed SD-4⁺ T-cell in the form that can be bound by DC-HIL, thereby extending the use of these toxin conjugates to the treatment T-cell cancers such as CTCL. No effect of DC-HIL-toxin on T_{reg} indicates that the toxin can be useful to deactivate effector T cell function while sparing immune suppression, thereby extending the use to the suppression of graft-versus-host disease, which hampers the therapeutic success of bone marrow transplantation. These and other aspects of the invention are set forth in detail below.

### 1. DC-HIL

### A. DC-HIL Structure

DC-HIL has a leader sequence (aa 1-19), a long extracellular domain (ECD, aa 20-499), a transmembrane domain (aa 500-523), and a cytoplasmic domain (aa 524-574). The ECD contains 11 potential N-glycosylation sites (NX(S/T)) and several putative O-glycosylation sites based on the stretch of proline-, serine-, and threonine-rich region, and a proline-rich region (aa 320-352) that presumably forms a hinge, as seen in proteins like IgA, which can mediate protein-protein interactions. Other functional motifs are an RGD sequence (aa 64-66), an integrin-binding sequence, and a KRFR (SEQ ID NO: 1) sequence (aa 23-26) that matches a heparin-binding motif composed of a stretch of basic residues (BBXB, where B represents a basic residue). The cytoplasmic tail contains an immunoreceptor tyrosine-based activation motif (ITAM (SEQ ID NO:2), YXXI, aa 529-532, where X represents all other amino acid residues) and two lysosomal targeting di-leucine motifs (LL, aa 548-549 and 566-567).

Human DC-HIL has a leader sequence (aa 1-19), a long extracellular domain (ECD, aa 20-495), a transmembrane domain (aa 496-518), and a cytoplasmic domain (aa 519-572). The ECD contains 11 potential N-glycosylation sites (NX(S/T)) and several putative O-glycosylation sites based on the stretch of proline-, serine-, and threonine-rich region, and a proline-rich region (aa 320-349) that presumably forms a hinge, as seen in proteins like IgA, which can mediate protein-protein interactions. Other functional motifs are an RGD sequence (aa 64-66), an integrin-binding sequence, and a KRFH (SEQ ID NO:3) sequence (aa 23-26) that matches a heparin-binding motif composed of a stretch of basic residues (BBXB, where B represents a basic residue). The cytoplasmic tail contains an immunoreceptor tyrosine-based activation motif (ITAM (SEQ ID NO:2), YXYI, aa 525-528) and two lysosomal targeting di-leucine motifs (LL, aa 516-517 and 562-563).

### B. Fragments

The present invention contemplates the use of various fragments of DC-HIL, in particular those that retain the ability to bind selectively to the syndecan-4 molecule expressed by activated T-cells, but not the syndecan-4 expressed by other cells, in including B-cell. A particular fragment of DC-HIL would include the Ig-like domain found in the extracellular portion of DC-HIL.

In particular, fragments of DC-HIL would thus include aa 259-319 in the human form:

(SEQ ID NO: 4)

LPIMFDVLIHDPSHFLNYSTINYKWSFGDNTGLFVSTNHTVNHTYVLNGT
FSLNLTVKAAA

and aa 256-319 in the mouse form:

(SEQ ID NO: 5)

LRDLPIVFDVLIHDPSHFLNDSAISYKWNFGDNTGLFVSNNHTLN HTYV
LNGTFNLNLTVQTAV

### C. Variants or Analogs of DC-HIL

Substitutional Variants. It also is contemplated in the present invention that variants or analogs of DC-HIL may also bind preferentially to syndecan-4 on activated T-cells. Sequence variants of DC-HIL, primarily making conservative substitutions, may provide improved compositions. Substitutional variants typically contain the exchange of one amino acid or amino acid analog for another at one or more sites within the molecule, and may be designed to modulate one or more properties of the molecule, in particular the affinity of the molecule for the target, without the loss of other functions or properties.

Altered Amino Acids. As shown above, peptides or proteins may employ modified, non-natural and/or unusual amino acids. A table of exemplary, but not limiting, modified, non-natural and/or unusual amino acids is provided herein below. Chemical synthesis may be employed to incorporated such amino acids into the peptides of interest.

TABLE 1
Modified, Non-Natural and Unusual Amino Acids

| Abbr. | Amino Acid |
|---|---|
| Aad | 2-Aminoadipic acid |
| BAad | 3-Aminoadipic acid |
| BAla | beta-alanine, beta-Amino-propionic acid |
| Abu | 2-Aminobutyric acid |
| 4Abu | 4-Aminobutyric acid, piperidinic acid |
| Acp | 6-Aminocaproic acid |
| Ahe | 2-Aminoheptanoic acid |
| Aib | 2-Aminoisobutyric acid |
| BAib | 3-Aminoisobutyric acid |
| Apm | 2-Aminopimelic acid |
| Dbu | 2,4-Diaminobutyric acid |
| Des | Desmosine |
| Dpm | 2,2′-Diaminopimelic acid |
| Dpr | 2,3-Diaminopropionic acid |
| EtGly | N-Ethylglycine |
| EtAsn | N-Ethylasparagine |
| Hyl | Hydroxylysine |
| AHyl | allo-Hydroxylysine |
| 3Hyp | 3-Hydroxyproline |
| 4Hyp | 4-Hydroxyproline |
| Ide | Isodesmosine |
| Aile | allo-Isoleucine |
| MeGly | N-Methylglycine, sarcosine |
| MeIle | N-Methylisoleucine |
| MeLys | 6-N-Methyllysine |
| MeVal | N-Methylvaline |
| Nva | Norvaline |
| Nle | Norleucine |
| Orn | Ornithine |

Mimetics. In addition to the variants discussed above, the present inventors also contemplate that structurally similar compounds may be formulated to mimic the key portions of DC-HIL, such as the Ig-like domain. Such compounds, which may be termed peptidomimetics, may be used in the same manner as the peptides of the invention and, hence, also are functional equivalents.

Certain mimetics that mimic elements of protein secondary and tertiary structure are described in Johnson et al. (1993). The underlying rationale behind the use of peptide mimetics is that the peptide backbone of proteins exists chiefly to orient amino acid side chains in such a way as to facilitate molecular interactions, such as those of antibody and/or antigen. A peptide mimetic is thus designed to permit molecular interactions similar to the natural molecule.

Some successful applications of the peptide mimetic concept have focused on mimetics of β-turns within proteins, which are known to be highly antigenic. Likely β-turn structure within a polypeptide can be predicted by computer-based algorithms, as discussed herein. Once the component amino acids of the turn are determined, mimetics can be constructed to achieve a similar spatial orientation of the essential elements of the amino acid side chains.

Other approaches have focused on the use of small, multidisulfide-containing proteins as attractive structural templates for producing biologically active conformations that mimic the binding sites of large proteins (Vita et al., 1998). A structural motif that appears to be evolutionarily conserved in certain toxins is small (30-40 amino acids), stable, and high permissive for mutation. This motif is composed of a β sheet and an α-helix bridged in the interior core by three disulfides.

β-II turns have been mimicked successfully using cyclic L-pentapeptides and those with D-amino acids (Weisshoff et al., 1999). Also, Johannesson et al. (1999) report on bicyclic tripeptides with reverse turn inducing properties.

Methods for generating specific structures have been disclosed in the art. For example, α-helix mimetics are disclosed in U.S. Pat. Nos. 5,446,128; 5,710,245; 5,840,833; and 5,859,184. Theses structures render the peptide or protein more thermally stable, also increase resistance to proteolytic degradation. Six, seven, eleven, twelve, thirteen and fourteen membered ring structures are disclosed.

Methods for generating conformationally restricted beta turns and beta bulges are described, for example, in U.S. Pat. Nos. 5,440,013; 5,618,914; and 5,670,155. β-turns permit changed side substituents without having changes in corresponding backbone conformation, and have appropriate termini for incorporation into peptides by standard synthesis procedures. Other types of mimetic turns include reverse and γ turns. Reverse turn mimetics are disclosed in U.S. Pat. Nos. 5,475,085 and 5,929,237, and gamma turn mimetics are described in U.S. Pat. Nos. 5,672,681 and 5,674,976.

### D. Purification of Proteins and Peptides

It may be desirable to purify proteins, peptides and conjugates (below) according to the present invention. Purification techniques are well known to those of skill in the art. These techniques typically involve chromatographic and electrophoretic techniques to achieve partial or complete purification (or purification to homogeneity). Analytical methods particularly suited to the preparation of a pure protein or peptide are ion-exchange chromatography, exclusion chromatography; polyacrylamide gel electrophoresis, isoelectric focusing. A particularly efficient method of purifying peptides or proteins is fast protein liquid chromatography or even HPLC.

Certain aspects of the present invention concern the purification, and in particular embodiments, the substantial purification, of a protein. The term “purified protein” as used herein, is intended to refer to a composition, isolatable from other components, wherein the protein is purified to any degree relative to its normally-obtainable state. A purified protein therefore also refers to a protein free from the environment in which it may normally occur.

Generally, “purified” will refer to a protein or peptide composition that has been subjected to fractionation to remove various other components, and which composition substantially retains its expressed biological activity. Where the term “substantially purified” is used, this designation will refer to a composition in which the protein or peptide forms the major component of the composition, such as constituting about 50%, about 60%, about 70%, about 80%, about 90%, about 95% or more of the composition by weight.

Various methods for quantifying the degree of purification of the protein or peptide will be known to those of skill in the art in light of the present disclosure. These include, for example, determining the specific activity of an active fraction, or assessing the amount of protein or peptide within a fraction by SDS/PAGE analysis. A preferred method for assessing the purity of a fraction is to calculate the specific activity of the fraction, to compare it to the specific activity of the initial extract, and to thus calculate the degree of purity, herein assessed by a “-fold purification number.” The actual units used to represent the amount of activity will, of course, be dependent upon the particular assay technique chosen to follow the purification and whether or not the peptide/protein exhibits a detectable activity.

Various techniques suitable for use in peptide/protein purification will be well known to those of skill in the art. These include, for example, precipitation with ammonium sulphate, PEG, antibodies and the like or by heat denaturation, followed by centrifugation; chromatography steps such as ion exchange, gel filtration, reverse phase, hydroxylapatite and affinity chromatography; isoelectric focusing; gel electrophoresis; and combinations of such and other techniques. As is generally known in the art, it is believed that the order of conducting the various purification steps may be changed, or that certain steps may be omitted, and still result in a suitable method for the preparation of a substantially purified peptide or protein.

High Performance Liquid Chromatography (HPLC) is characterized by a very rapid separation with extraordinary resolution of peaks. This is achieved by the use of very fine particles and high pressure to maintain an adequate flow rate. Separation can be accomplished in a matter of minutes, or at most an hour. Moreover, only a very small volume of the sample is needed because the particles are so small and close-packed that the void volume is a very small fraction of the bed volume. Also, the concentration of the sample need not be very great because the bands are so narrow that there is very little dilution of the sample.

Gel chromatography, or molecular sieve chromatography, is a special type of partition chromatography that is based on molecular size. The theory behind gel chromatography is that the column, which is prepared with tiny particles of an inert substance that contain small pores, separates larger molecules from smaller molecules as they pass through or around the pores, depending on their size. As long as the material of which the particles are made does not adsorb the molecules, the sole factor determining rate of flow is the size. Hence, molecules are eluted from the column in decreasing size, so long as the shape is relatively constant. Gel chromatography is unsurpassed for separating molecules of different size because separation is independent of all other factors such as pH, ionic strength, temperature, etc. There also is virtually no adsorption, less zone spreading and the elution volume is related in a simple matter to molecular weight.

Affinity Chromatography is a chromatographic procedure that relies on the specific affinity between a substance to be isolated and a molecule that it can specifically bind to. This is a receptor-ligand type interaction. The column material is synthesized by covalently coupling one of the binding partners to an insoluble matrix. The column material is then able to specifically adsorb the substance from the solution. Elution occurs by changing the conditions to those in which binding will not occur (alter pH, ionic strength, temperature, etc.).

A particular type of affinity chromatography useful in the purification of carbohydrate containing compounds is lectin affinity chromatography. Lectins are a class of substances that bind to a variety of polysaccharides and glycoproteins. Lectins are usually coupled to agarose by cyanogen bromide. Conconavalin A coupled to Sepharose was the first material of this sort to be used and has been widely used in the isolation of polysaccharides and glycoproteins other lectins that have been include lentil lectin, wheat germ agglutinin which has been useful in the purification of N-acetyl glucosaminyl residues and Helix pomatia lectin. Lectins themselves are purified using affinity chromatography with carbohydrate ligands. Lactose has been used to purify lectins from castor bean and peanuts; maltose has been useful in extracting lectins from lentils and jack bean; N-acetyl-D galactosamine is used for purifying lectins from soybean; N-acetyl glucosaminyl binds to lectins from wheat germ; D-galactosamine has been used in obtaining lectins from clams and L-fucose will bind to lectins from lotus.

The matrix should be a substance that itself does not adsorb molecules to any significant extent and that has a broad range of chemical, physical and thermal stability. The ligand should be coupled in such a way as to not affect its binding properties. The ligand should also provide relatively tight binding. And it should be possible to elute the substance without destroying the sample or the ligand. One of the most common forms of affinity chromatography is immunoaffinity chromatography. The generation of antibodies that would be suitable for use in accord with the present invention is discussed below.

### 2. CONJUGATES

The present invention, one aspect, provides for conjugates between DC-HIL or syndecan-4-binding fragments thereof and a toxin that can kill or impair the function of an activated T-cell that expresses syndecan-4 on its surface. The following discussion of toxins and linking technologies is exemplary and in no way limiting with respect to implementation of this embodiment of the invention.

A. Toxins

A toxin is a poisonous substance produced by living cells or organisms that is active at very low concentrations. Toxins can be small molecules, peptides, or proteins and are capable of causing disease on contact or absorption with body tissues by interacting with biological macromolecules such as enzymes or cellular receptors.

Biotoxins vary greatly in purpose and mechanism, and can be highly complex (the venom of the cone snail contains dozens of small proteins, each targeting a specific nerve channel or receptor), or relatively small protein. Examples include cyantoxins, produced by cyanobacteria, hemotoxins, which target and destroy red blood cells, and are transmitted through the bloodstream, necrotoxins, which cause necrosis in the cells they encounter and destroy all types of tissue, neurotoxins, which primarily affect the nervous systems of animals, and plant toxins.

Ricin. Ricin is a protein toxin that is extracted from the castor bean (Ricinus communis), and is poisonous if inhaled, injected, or ingested, acting as a toxin by the inhibition of protein synthesis. While there is no known antidote, the US military has developed a vaccine. Symptomatic and supportive treatment is available. Long term organ damage is likely in survivors. Ricin causes severe diarrhea and victims can die of shock. Abrin is a similar toxin. Deaths caused by ingestion of castor oil plant seeds is rare. Eight beans are considered toxic for an adult. A solution of saline and glucose has been used to treat ricin overdose.

Ricin consists of two distinct protein chains (almost 30 kDa each) that are linked to each other by a disulfide bond. Ricin A is an N-glycoside hydrolase that targets and depurinates an adenine base in the 28S rRNA molecule of the ribosome, resulting in an inhibition of protein biosynthesis. Ricin B is a lectin that binds galactosyl residues and is important in assisting ricin A's entry into a cell by binding with a cell surface component. Many plants such as barley have the A chain but not the B chain. People do not get sick from eating large amounts of such products, as ricin A alone is of extremely low toxicity.

Diptheria toxin. The diphtheria toxin causes the death eucaryotic cells and tissues by inhibition protein synthesis in the cells. Although the toxin is responsible for the lethal symptoms of the disease, the virulence of C. diphtheriae cannot be attributed to toxigenicity alone, since a distinct invasive phase apparently precedes toxigenesis. However, it has not been ruled out that the diphtheria toxin plays an essential role in the colonization process due to short-range effects at the colonization site.

Botulinum toxin. Botulinum toxin is a neurotoxin protein produced by the bacterium Clostridium botulinum. It is one of the most poisonous naturally occurring substances in the world, and it is the most toxic protein. Though it is highly toxic, it is used in minute doses both to treat painful muscle spasms, and as a cosmetic treatment in some parts of the world. It is sold under the brandnames Myobloc, Botox and Dysport.

Saporin. Saporin is a protein that is useful in biological research applications, especially studies of behavior. Saporin is a so-called ribosome-inactivating protein (RIP), due to its N-glycosidase activity, from the seeds of Saponaria officinalis (common name: soapwort). It was first described by Fiorenzo Stirpe and his colleagues in 1983 in an article that illustrated the unusual stability of the protein.

B. Linkers

Any of a wide variety of linkers may be utilized to effect the joinder of proteins/peptide of DC-HIL to toxins. Certain linkers will generally be preferred over other linkers, based on differing pharmacologic characteristics and capabilities.

Cross-linking reagents are used to form molecular bridges that tie together functional groups of two molecules. Any linking/coupling agents known to those of skill in the art can be used to combine to molecules of the present invention, such as, avidin-biotin linkages, amide linkages, ester linkages, thioester linkages, ether linkages, thioether linkages, phosphoester linkages, phosphoramide linkages, anhydride linkages, disulfide linkages, ionic and hydrophobic interactions.

TABLE 2
HETERO-BIFUNCTIONAL CROSS-LINKERS

| Linker | Reactive Toward | Advantages and Applications | Spacer Arm Length |
|---|---|---|---|
| SMPT | Primary amines | Greater stability | 11.2 A |
| | Sulfhydryls | | |
| SPDP | Primary amines | Thiolation | 6.8 A |
| | Sulfhydryls | Cleavable cross-linking | |
| LC-SPDP | Primary amines | Extended spacer arm | 15.6 A |
| | Sulfhydryls | | |
| Sulfo-LC-SPDP | Primary amines | Extended spacer arm | 15.6 A |
| | Sulfhydryls | Water-soluble | |
| SMCC | Primary amines | Stable maleimide reactive group | 11.6 A |
| | Sulfhydryls | Enzyme-antibody conjugation | |
| | | Hapten-carrier protein conjugation | |
| Sulfo-SMCC | Primary amines | Stable maleimide reactive group | 11.6 A |
| | Sulfhydryls | Water-soluble | |
| | | Enzyme-antibody conjugation | |
| MBS | Primary amines | Enzyme-antibody conjugation | 9.9 A |
| | Sulfhydryls | Hapten-carrier protein conjugation | |
| Sulfo-MBS | Primary amines | Water-soluble | 9.9 A |
| | Sulfhydryls | | |
| SIAB | Primary amines | Enzyme-antibody conjugation | 10.6 A |
| | Sulfhydryls | | |
| Sulfo-SIAB | Primary amines | Water-soluble | 10.6 A |
| | Sulfhydryls | | |
| SMPB | Primary amines | Extended spacer arm | 14.5 A |
| | Sulfhydryls | Enzyme-antibody conjugation | |
| Sulfo-SMPB | Primary amines | Extended spacer arm | 14.5 A |
| | Sulfhydryls | Water-soluble | |
| EDC/Sulfo-NHS | Primary amines | Hapten-Carrier conjugation | 0 |
| | Carboxyl groups | | |
| ABH | Carbohydrates | Reacts with sugar groups | 11.9 A |
| | Nonselective | | |

An exemplary hetero-bifunctional cross-linker contains two reactive groups: one reacting with primary amine group (e.g., N-hydroxy succinimide) and the other reacting with a thiol group (e.g., pyridyl disulfide, maleimides, halogens, etc.). Through the primary amine reactive group, the cross-linker may react with the lysine residue(s) of one protein (e.g., the selected antibody or fragment) and through the thiol reactive group, the cross-linker, already tied up to the first protein, reacts with the cysteine residue (free sulfhlydryl group) of the other protein (e.g., the selective agent).

It is particular that a cross-linker having reasonable stability in blood will be employed. Numerous types of disulfide-bond containing linkers are known that can be successfully employed to conjugate targeting and therapeutic/preventative agents. Linkers that contain a disulfide bond that is sterically hindered may prove to give greater stability in vivo, preventing release of the targeting peptide prior to reaching the site of action. These linkers are thus one group of linking agents.

Another cross-linking reagent is SMPT, which is a bifunctional cross-linker containing a disulfide bond that is “sterically hindered” by an adjacent benzene ring and methyl groups. It is believed that steric hindrance of the disulfide bond serves a function of protecting the bond from attack by thiolate anions such as glutathione which can be present in tissues and blood, and thereby help in preventing decoupling of the conjugate prior to the delivery of the attached agent to the target site.

The SMPT cross-linking reagent, as with many other known cross-linking reagents, lends the ability to cross-link functional groups such as the SH of cysteine or primary amines (e.g., the epsilon amino group of lysine). Another possible type of cross-linker includes the hetero-bifunctional photoreactive phenylazides containing a cleavable disulfide bond such as sulfosuccinimidyl-2-(p-azido salicylamido) ethyl-1,3′-dithiopropionate. The N-hydroxy-succinimidyl group reacts with primary amino groups and the phenylazide (upon photolysis) reacts non-selectively with any amino acid residue.

In addition to hindered cross-linkers, non-hindered linkers also can be employed in accordance herewith. Other useful cross-linkers, not considered to contain or generate a protected disulfide, include SATA, SPDP and 2-iminothiolane (Wawrzynczak & Thorpe, 1986). The use of such cross-linkers is well understood in the art. Another embodiment involves the use of flexible linkers.

U.S. Pat. No. 4,680,338, describes bifunctional linkers useful for producing conjugates of ligands with amine-containing polymers and/or proteins, especially for forming antibody conjugates with chelators, drugs, enzymes, detectable labels and the like. U.S. Pat. Nos. 5,141,648 and 5,563,250 disclose cleavable conjugates containing a labile bond that is cleavable under a variety of mild conditions. This linker is particularly useful in that the agent of interest may be bonded directly to the linker, with cleavage resulting in release of the active agent. Preferred uses include adding a free amino or free sulfhydryl group to a protein, such as an antibody, or a drug.

U.S. Pat. No. 5,856,456 provides peptide linkers for use in connecting polypeptide constituents to make fusion proteins, e.g., single-chain antibodies. The linker is up to about 50 amino acids in length, contains at least one occurrence of a charged amino acid (preferably arginine or lysine) followed by a proline, and is characterized by greater stability and reduced aggregation. U.S. Pat. No. 5,880,270 discloses aminooxy-containing linkers useful in a variety of immunodiagnostic and separative techniques.

Peptide linkers that include a cleavage site for an enzyme preferentially located or active within a tumor environment also are contemplated. Exemplary forms of such peptide linkers are those that are cleaved by urokinase, plasmin, thrombin, Factor IXa, Factor Xa, or a metallaproteinase, such as collagenase, gelatinase, or stromelysin.

### 3. T-CELL INFLAMMATORY DISORDERS

### A. Transplant Rejection

Host-versus-graft. Host-versus-graft rejection is defined as the normal lymphocyte mediated reactions of a host against allogeneic or xenogeneic cells acquired as a graft or otherwise, which lead to damage or/and destruction of the grafted cells. The opposite of graft-versus-host reaction. The common basis of graft rejection.

“Acute rejection” is generally acknowledged to be mediated by T-cell responses to proteins from the donor organ which differ from those found in the recipient. Unlike antibody-mediated hyperacute rejection, development of T-cell responses first occurs several days after a transplant if the patient is not taking immunosuppressant drugs. Since the development of powerful immunosuppressive drugs such as cyclosporin, tacrolimus and rapamycin, the incidence of acute rejection has been greatly decreased, however, organ transplant recipients can develop acute rejection episodes months to years after transplantation. Acute rejection episodes can destroy the transplant if it is not recognized and treated appropriately. Episodes occur in around 60-75% of first kidney transplants, and 50-60% of liver transplants. A single episode is not a cause for concern if recognised and treated promptly and rarely leads to organ failure, but recurrent episodes are associated with chronic rejection of grafts. The bulk of the immune system response is to the Major Histocompatibility Complex (MHC) proteins. MHC proteins are involved in the presentation of foreign antigens to T-cells, and receptors on the surface of the T-cell (TCR) are uniquely suited to recognition of proteins of this type. MHC are highly variable between individuals, and therefore the T-cells from the host recognize the foreign MHC with a very high frequency leading to powerful immune responses that cause rejection of transplanted tissue. Identical twins and cloned tissue are MHC matched, and are therefore not subject to T-cell mediated rejection.

The diagnosis of acute rejection relies on the clinical data including patient signs and symptoms, laboratory testing and ultimately a liver biopsy. The biopsy is interpreted by a pathologist who notes changes in the tissue that suggest rejection. Histologically acute rejection is characterized by three main features. First, a predominately T-cell rich lymphocytic infiltrate is often present and may be accompanied by a heterogeneous infiltrate including eosinophils, scattered plasma cells and neutrophils. Of note, an abundance of eosinophils within the mixed infiltrate is a helpful feature of acute rejection. Secondly, evidence of injury to the bile ducts is often seen, manifested by the presence of intrepithelial lymphocytes and loss of epithelial cell polarity. Lastly, injury to the vessels may be seen as endothelialitis. Typically this involves portal vein branches, but may include central veins and sinusoids. For a pathologist who evaluates biopsies for liver disease following transplantation, it is important to be aware of the disorders that commonly occur in this setting and their histologic differences. These include autoimmune hepatitis, which will often have a large number of plasma cells; post-transplant lymphoproliferative disorder with its characteristic monotonous infiltrate and primary biliary cirrhosis which may have focal injury to bile ducts unlike the more monotonous process seen in acute rejection.

Rejection is prevented with a combination of drugs including Calcineurin inhibitors such as Cyclosporin and Tacrolimus; mTOR inhibitors such as Sirolimus and Everolimus; anti-proliferatives such as Azathioprine and Mycophenolic acid; corticosteroids such as Prednisolone and Hydrocortisone; and antibodies such as Basiliximab, Daclizumab, Anti-thymocyte globulin (ATG), and Anti-lymphocyte globulin (ALG)

Generally, a triple therapy regimen of a calcineurin inhibitor, an anti-proliferative, and a corticosteroid is used, although local protocols vary. Antibody inductions can be added to this, especially for high-risk patients and in the U.S. mTOR inhibitors can be used to provide calcineurin-inhibitor or steroid-free regimes in selected patients.

An FDA approved immune function test from Cylex has shown effectiveness in minimizing the risk of infection and rejection in post-transplant patients by enabling doctors to tailor immunosuppressant drug regimens. By keeping a patient's immune function within a certain window, doctors could adjust drug levels to prevent organ rejection while avoiding infection. Such information could help physicians reduce the use of immunosuppressive drugs, lowering drug therapy expenses while reducing the morbidity associated with biopsies, improve the daily life of transplant patients, and could prolong the life of the transplanted organ.

Acute rejection is normally treated initially with a short course of high-dose methylprednisolone, which is usually sufficient to treat successfully. If this is not enough, the course can be repeated or ATG can be given. Acute rejection refractory to these treatments may require plasma exchanges to remove antibodies to the transplant. The monoclonal anti-T-cell antibody OKT3 was formerly used in the prevention of rejection, and is occasionally used in treatment of severe acute rejection, but has fallen out of common use due to the severe cytokine release syndrome and late post-transplant lymphoproliferative disorder, which are both commonly associated with use of OKT3; in the United Kingdom it is available on a named-patient use basis only.

Acute rejection usually begins after the first week of transplantation, and most likely occurs to some degree in all transplants (except between identical twins). It is caused by mismatched HLA antigens that are present on all cells. HLA antigens are polymorphic therefore the chance of a perfect match is extremely rare. The reason that acute rejection occurs a week after transplantation is because the T-cells involved in rejection must differentiate and the antibodies in response to the allograft must be produced before rejection is initiated. These T-cells cause the grafT-cells to lyse or produce cytokines that recruit other inflammatory cells, eventually causing necrosis of allograft tissue. Endothelial cells in vascularized grafts such as kidneys are some of the earliest victims of acute rejection. Damage to the endothelial lining is an early predictor of irreversible acute graft failure. The risk of acute rejection is highest in the first 3 months after transplantation, and is lowered by immunosuppressive agents in maintenance therapy. The onset of acute rejection is combatted by episodic treatment.

### B. Skin Conditions

Contact hypersensitivity/dermatitis. Contact dermatitis (contact hypersensitivity) is a term for a skin reaction resulting from exposure to allergens (allergic contact dermatitis) or irritants (irritant contact dermatitis). Phototoxic dermatitis occurs when the allergen or irritant is activated by sunlight. Contact dermatitis is a localized rash or irritation of the skin caused by contact with a foreign substance. Only the superficial regions of the skin are affected in contact dermatitis. Inflammation of the affected tissue is present in the epidermis (the outermost layer of skin) and the outer dermis (the layer beneath the epidermis). Unlike contact urticaria, in which a rash appears within minutes of exposure and fades away within minutes to hours, contact dermatitis takes days to fade away. Even then, contact dermatitis fades only if the skin no longer comes in contact with the allergen or irritant. Contact dermatitis results in large, burning, and itchy rashes, and these can take anywhere from several days to weeks to heal. Chronic contact dermatitis can develop when the removal of the offending agent no longer provides expected relief. In North/South America, the most common causes of allergic contact dermatitis are plants of the Toxicodendron genus: poison ivy, poison oak, and poison sumac. Common causes of irritant contact dermatitis are harsh (highly alkaline) soaps, nickel, detergents, and cleaning products and rubbers.

There are three types of contact dermatitis: irritant contact, allergic contact, and photocontact dermatitis. Photocontact dermatitis is divided into two categories: phototoxic and photoallergic. Chemical irritant contact dermatitis is either acute or chronic, which is usually associated with strong and weak irritants respectively (HSE MS24).

Common chemical irritants implicated include solvents (alcohol, xylene, turpentine, esters, acetone, ketones, and others); metalworking fluids (neat oils, water-based metalworking fluids with surfactants); latex; kerosene; ethylene oxide; surfactants in topical medications and cosmetics (sodium lauryl sulfate); alkalies (drain cleaners, strong soap with lye residues).

Physical irritant contact dermatitis is a less researched form of ICD (Maurice-Jones et al) due to its various mechanisms of action and a lack of a test for its diagnosis. A complete patient history combined with negative allergic patch testing is usually necessary to reach a correct diagnosis. The simplest form of PICD results from prolonged rubbing, although the diversity of implicated irritants is far wider. Examples include paper friction, fiberglass, and scratchy clothing.

Many plants cause ICD by directly irritating the skin. Some plants act through their spines or irritant hairs. Some plant such as the buttercup, spurge, and daisy act by chemical means. The sap of these plants contains a number of alkaloids, glycosides, saponins, anthraquinones, and (in the case of plant bulbs) irritant calcium oxalate crystals—all of which can cause CICD (Mantle et al., 2001).

Allergic Contact Dermatitis (ACD) is a condition that is the manifestation of an allergic response caused by contact with a substance. Although less common than ICD, ACD is accepted to be the most prevalent form of immunotoxicity found in humans. By its allergic nature, this form of contact dermatitis is a hypersensitive reaction that is atypical within the population. The mechanisms by which these reactions occur are complex, with many levels of fine control. Their immunology centers around the interaction of immunoregulatory cytokines and discrete subpopulations of T lymphocytes.

ACD arises as a result of two essential stages: an induction phase, which primes and sensitizes the immune system for an allergic response, and an elicitation phase, in which this response is triggered. As such, ACD is termed a Type IV delayed hypersensitivity reaction involving a cell-mediated allergic response. Contact allergens are essentially soluble haptens (low in molecular weight) and, as such, have the physico-chemical properties that allow them to cross the stratum corneum of the skin. They can only cause their response as part of a complete antigen, involving their association with epidermal proteins forming hapten-protein conjugates. This, in turn, requires them to be protein-reactive.

Sometimes termed “photoaggravated,” and divided into two categories, phototoxic and photoallergic, PCD is the eczematous condition which is triggered by an interaction between an otherwise unharmful or less harmful substance on the skin and ultraviolet light (320-400 nm UVA), therefore manifesting itself only in regions where the sufferer has been exposed to such rays. Without the presence of these rays, the photosensitiser is not harmful. For this reason, this form of contact dermatitis is usually associated only with areas of skin which are left uncovered by clothing. The mechanism of action varies from toxin to toxin, but is usually due to the production of a photoproduct. Toxins which are associated with PCD include the psoralens. Psoralens are in fact used therapeutically for the treatment of psoriasis, eczema and vitiligo. Photocontact dermatitis is another condition where the distinction between forms of contact dermatitis is not clear cut. Immunological mechanisms can also play a part, causing a response similar to ACD.

Allergic dermatitis is usually confined to the area where the trigger actually touched the skin, whereas irritant dermatitis may be more widespread on the skin. Symptoms of both forms include the following:
Red rash—this is the usual reaction. The rash appears immediately in irritant contact dermatitis; in allergic contact dermatitis, the rash sometimes does not appear until 24-72 hours after exposure to the allergen.
Blisters or wheals—blisters, wheals (welts), and urticaria (hives) often form in a pattern where skin was directly exposed to the allergen or irritant.
Itchy, burning skin—irritant contact dermatitis tends to be more painful than itchy, while allergic contact dermatitis often itches.
   While either form of contact dermatitis can affect any part of the body, irritant contact dermatitis often affects the hands, which have been exposed by resting in or dipping into a container (sink, pail, tub) containing the irritant.

Immediately after exposure to a known allergen or irritant, wash with soap and cool water to remove or inactivate most of the offending substance. Weak acid solutions, such as lemon juice, vinegar, can be used to counteract the effects of dermatitis contracted by exposure to basic irritants. If blistering develops, cold moist compresses applied for 30 minutes 3 times a day can offer relief. Calamine lotion and cool colloidal oatmeal baths may relieve itching. Oral antihistamines such as diphenhydramine (Benadryl, Ben-Allergin) can also relieve itching. For mild cases that cover a relatively small area, hydrocortisone cream in nonprescription strength may be sufficient. Avoid scratching, as this can cause secondary infections.

If the rash does not improve or continues to spread after 2-3 of days of self-care, or if the itching and/or pain is severe, the patient should contact a dermatologist or other physician. Medical treatment usually consists of lotions, creams, or oral medications. A corticosteroid medication similar to hydrocortisone may be prescribed to combat inflammation in a localized area. This medication may be applied to your skin as a cream or ointment. If the reaction covers a relatively large portion of the skin or is severe, a corticosteroid in pill or injection form may be prescribed. Prescription antihistamines may be given if nonprescription strengths are inadequate.

Since contact dermatitis relies on an irritant or an allergen to initiate the reaction, it is important for the patient to identify the responsible agent and avoid it. This can be accomplished by having patch tests, a method commonly known as allergy testing. The patient must know where the irritant or allergen is found to be able to avoid it. It is important to also note that chemicals sometimes have several different names.^{[14]}

Atopic dermatitis. Atopic dermatitis, also known as atopic eczema, is an atopic, hereditary, and non-contagious skin disease characterized by chronic inflammation of the skin. The skin of a patient with atopic dermatitis reacts abnormally and easily to irritants, food, and environmental allergens and becomes red, flaky and very itchy. It also becomes vulnerable to surface infections caused by bacteria. The skin on the flexural surfaces of the joints (for example inner sides of elbows and knees) are most commonly affected regions in people.

Since the twentieth century, many mucosal inflammatory disorders have become dramatically more common; atopic eczema (AE) is a classic example of such a disease. It now affects 10-20% of children and 1-3% of adults in industrialized countries, and its prevalence there has more than doubled in the past thirty years.

Although it is an inherited disease, eczema is primarily aggravated by contact with or intake of allergens. It can also be influenced by other “hidden” factors such as stress or fatigue. Atopic eczema consists of chronic inflammation; it often occurs in people with a history of allergy disorders such as asthma or hay fever.

Atopic dermatitis often occurs together with other atopic diseases like hay fever, asthma and conjunctivitis. It is a familial and chronic disease and its symptoms can increase or disappear over time. Atopic dermatitis in older children and adults is often confused with psoriasis. Atopic dermatitis afflicts humans, particularly young children; it is also a well-characterized disease in domestic dogs. Although there is no cure for atopic eczema, and its causes not well understood, it can be treated very effectively in the short term through a combination of prevention (learning what triggers the allergic reactions) and drug therapy.

The primary treatment involves prevention, which includes avoiding or minimizing contact with (or intake of) known allergens. Once that has been established, topical treatments can be used. Topical treatments focus on reducing both the dryness and inflammation of the skin.

To combat the severe dryness associated with eczema, a high-quality, dermatologist approved moisturizer should be used daily. Moisturizers should not have any ingredients that may further aggravate the condition. Moisturizers are especially effective if applied within 5-10 minutes after bathing.

Most commercial soaps wash away the oils produced by the skin that normally serve to prevent drying. Using a soap substitute such as aqueous cream helps keep the skin moisturized. A non-soap cleanser can be purchased usually at a local drug store. Showers should be kept short and at a lukewarm/moderate temperature.

If moisturizers on their own don't help and the eczema is severe, a doctor may prescribe topical steroid ointments or creams. Steroid creams have traditionally been considered the most effective method of treating severe eczema. Disadvantages of using steroid creams include stretch marks and thinning of the skin. Higher-potency steroid creams must not be used on the face or other areas where the skin is naturally thin; usually a lower-potency steroid is prescribed for sensitive areas. Along with creams, antibiotics are often prescribed if an infection is suspected. If the eczema is especially severe, a doctor may prescribe prednisone or administer a shot of cortisone. If the eczema is mild, over-the-counter hydrocortisone can be purchased at the local drugstore.

The immunosuppressant Tacrolimus or pimecrolimus can be used as a topical preparation in the treatment of severe atopic dermatitis instead of traditional steroid creams. However, there can be unpleasant side effects in some patients such as intense stinging or burning. Some alternative medicines may (illegally) contain very strong steroids. Others are completely harmless, such as Oolong tea.

A more novel form of treatment involves exposure to broad or narrow-band ultraviolet light. UV radiation exposure has been found to have a localized immunomodulatory effect on affected tissues, and may be used to decrease the severity and frequency of flares. In particular, some researchers have suggested that the usage of UVA1 is more effective in treating acute flares, whereas narrow-band UVB is more effective in long-term management scenarios. However, UV radiation has also been implicated in various types of skin cancer, and thus UV treatment is not without risk.

If ultraviolet light therapy is employed, initial exposure should be no longer than 5-10 minutes, depending on skin type. UV therapy should only be moderate, and special care should be taken to avoid sunburn (sunburn will only aggravate the eczema). It does not necessarily have to be administered in a hospital, it can be done at a tanning salon or in natural sunlight, so as long as it is performed under the direction and supervision of a dermatologist.

Many of the same types of treatment are used in domestic dogs with atopic dermatitis. In addition, domestic dogs may be successfully managed with allergen-specific immunotherapy; many are treated with low-dose cyclosporine lipid emulsion.

Psoriasis. Psoriasis is a disease which affects the skin and joints. It commonly causes red scaly patches to appear on the skin. The scaly patches caused by psoriasis, called psoriatic plaques, are areas of inflammation and excessive skin production. Skin rapidly accumulates at these sites and takes a silvery-white appearance. Plaques frequently occur on the skin of the elbows and knees, but can affect any area including the scalp and genitals. Psoriasis is hypothesized to be immune-mediated and is not contagious.

The disorder is a chronic recurring condition which varies in severity from minor localised patches to complete body coverage. Fingernails and toenails are frequently affected (psoriatic nail dystrophy)—and can be seen as an isolated finding. Psoriasis can also cause inflammation of the joints, which is known as psoriatic arthritis. Ten to fifteen percent of people with psoriasis have psoriatic arthritis. The symptoms of psoriasis can manifest in a variety of forms. Variants include plaque, pustular, guttate and flexural psoriasis.

The cause of psoriasis is not known, but it is believed to have a genetic component. Several factors are thought to aggravate psoriasis. These include stress, excessive alcohol consumption, and smoking. Individuals with psoriasis may suffer from depression and loss of self-esteem. As such, quality of life is an important factor in evaluating the severity of the disease. There are many treatments available but because of its chronic recurrent nature psoriasis is a challenge to treat.

There are two main hypotheses about the process that occurs in the development of the disease. The first considers psoriasis as primarily a disorder of excessive growth and reproduction of skin cells. The problem is simply seen as a fault of the epidermis and its keratinocytes. The second hypothesis sees the disease as being an immune-mediated disorder in which the excessive reproduction of skin cells is secondary to factors produced by the immune system. T-cells (which normally help protect the body against infection) become active, migrate to the dermis and trigger the release of cytokines (tumor necrosis factor-alpha (TNFα), in particular) which cause inflammation and the rapid production of skin cells. It is not known what initiates the activation of the T-cells.

Psoriasis is a fairly idiosyncratic disease. The majority of people's experience of psoriasis is one in which it may worsen or improve for no apparent reason. Studies of the factors associated with psoriasis tend to be based on small (usually hospital based) samples of individuals. These studies tend to suffer from representative issues, and an inability to tease out causal associations in the face of other (possibly unknown) intervening factors. Conflicting findings are often reported. Nevertheless, the first outbreak is sometimes reported following stress (physical and mental), skin injury, and streptococcal infection. Conditions that have been reported as accompanying a worsening of the disease include infections, stress, and changes in season and climate. Certain medicines, including lithium salt and beta blockers, have been reported to trigger or aggravate the disease. Excessive alcohol consumption, smoking and obesity may exacerbate psoriasis or make the management of the condition difficult.

There can be substantial variation between individuals in the effectiveness of specific psoriasis treatments. Because of this, dermatologists often use a trial-and-error approach to finding the most appropriate treatment for their patient. The decision to employ a particular treatment is based on the type of psoriasis, its location, extent and severity. The patient's age, gender, quality of life, comorbidities, and attitude toward risks associated with the treatment are also taken into consideration.

Medications with the least potential for adverse reactions are preferentially employed. If the treatment goal is not achieved then therapies with greater potential toxicity may be used. Medications with significant toxicity are reserved for severe unresponsive psoriasis. This is called the psoriasis treatment ladder. As a first step, medicated ointments or creams, called topical treatments, are applied to the skin. If topical treatment fails to achieve the desired goal then the next step would be to expose the skin to ultraviolet (UV) radiation. This type of treatment is called phototherapy. The third step involves the use of medications which are taken internally by pill or injection. This approach is called systemic treatment.

Over time, psoriasis can become resistant to a specific therapy. Treatments may be periodically changed to prevent resistance developing (tachyphylaxis) and to reduce the chance of adverse reactions occurring. This is called treatment rotation.

Bath solutions and moisturizers help soothe affected skin and reduce the dryness which accompanies the build-up of skin on psoriatic plaques. Medicated creams and ointments applied directly to psoriatic plaques can help reduce inflammation, remove built-up scale, reduce skin turn over, and clear affected skin of plaques. Ointment and creams containing coal tar, dithranol (anthralin), corticosteroids like Topicort Desoximetasone), vitamin D₃ analogues (for example, calcipotriol), and retinoids are routinely used. Argan oil has also been used with some promising results. The mechanism of action of each is probably different but they all help to normalise skin cell production and reduce inflammation. Activated vitamin D and its analogues are highly effective inhibitors of skin cell proliferation.

The disadvantages of topical agents are variably that they can often irritate normal skin, can be time consuming and awkward to apply, cannot be used for long periods, can stain clothing or have a strong odour. As a result, it is sometimes difficult for people to maintain the regular application of these medications. Abrupt withdrawal of some topical agents, particularly corticosteroids, can cause an aggressive recurrence of the condition. This is known as a rebound of the condition.

Some topical agents are used in conjunction with other therapies, especially phototherapy. It has long been recognized that daily, short, non-burning exposure to sunlight helped to clear or improve psoriasis. Niels Finsen was the first physician to investigate the therapeutic effects of sunlight scientifically and to use sunlight in clinical practice. This became known as phototherapy.

Sunlight contains many different wavelengths of light. It was during the early part of the 20th century that it was recognised that for psoriasis the therapeutic property of sunlight was due to the wavelengths classified as ultraviolet (UV) light. Ultraviolet wavelengths are subdivided into UVA (380-315 nm) UVB (315-280 nm), and UVC (<280 nm). Ultraviolet B (UVB) (315-280 nm) is absorbed by the epidermis and has a beneficial effect on psoriasis. Narrowband UVB (311 to 312 nm), is that part of the UVB spectrum that is most helpful for psoriasis. Exposure to UVB several times per week, over several weeks can help people attain a remission from psoriasis.

Ultraviolet light treatment is frequently combined with topical (coal tar, calcipotriol) or systemic treatment (retinoids) as there is a synergy in their combination. The Ingram regime, involves UVB and the application of anthralin paste. The Goeckerman regime combines coal tar ointment with UVB.

Psoralen and ultraviolet A phototherapy (PUVA) combines the oral or topical administration of psoralen with exposure to ultraviolet A (UVA) light. Precisely how PUVA works is not known. The mechanism of action probably involves activation of psoralen by UVA light which inhibits the abnormally rapid production of the cells in psoriatic skin. There are multiple mechanisms of action associated with PUVA, including effects on the skin immune system. PUVA is associated with nausea, headache, fatigue, burning, and itching. Long-term treatment is associated with squamous-cell and melanoma skin cancers.

Psoriasis which is resistant to topical treatment and phototherapy is treated by medications that are taken internally by pill or injection. This is called systemic treatment. Patients undergoing systemic treatment are required to have regular blood and liver function tests because of the toxicity of the medication. Pregnancy must be avoided for the majority of these treatments. Most people experience a recurrence of psoriasis after systemic treatment is discontinued.

The three main traditional systemic treatments are methotrexate, cyclosporine and retinoids. Methotrexate and cyclosporine are immunosupressant drugs; retinoids are synthetic forms of vitamin A. Other additional drugs, not specifically licensed for psoriasis, have been found to be effective. These include the antimetabolite tioguanine, the cytotoxic agent hydroxyurea, sulfasalazine, the immunosupressants mycophenolate mofetil, azathioprine and oral tacrolimus. These have all been used effectively to treat psoriasis when other treatments have failed. Although not licensed in many other countries fumaric acid esters have also been used to treat severe psoriasis in Germany for over 20 years.

Biologics are manufactured proteins that interrupt the immune process involved in psoriasis. Unlike generalised immunosuppressant therapies such as methotrexate, biologics focus on specific aspects of the immune function leading to psoriasis. These drugs (interleukin antagonists) are relatively new, and their long-term impact on immune function is unknown. They are very expensive and only suitable for very few patients with psoriasis. Ustekinumab (IL-12 and IL-23 blocker) shows hopeful results for psoriasis therapy.

A new natural systemic option, XP-828L, for mild to moderate psoriasis relief has been developed by a Canadian life science and technology company. This oral product with clinically proven efficacy and safety is extracted through a patented process from whey and has immuno-modulatory effects. Antibiotics are not indicated in routine treatment of psoriasis. However, antibiotics may be employed when an infection, such as that caused by the bacteria Streptococcus, triggers an outbreak of psoriasis, as in certain cases of guttate psoriasis.

### C. Autoimmune Disorders

A variety of autoimmune disorders may be treated in accordance with the present invention, including ankylosing spondylitis, psoriatic arthritis, enteropathic arthritis, reactive arthritis, undifferentiated spondyloarthropathy, juvenile spondyloarthropathy, Behcet's disease, enthesitis, ulcerative colitis, Crohn's disease, irritable bowel syndrome, inflammatory bowel disease, fibromyalgia, chronic fatigue syndrome, pain conditions associated with systemic inflammatory disease, systemic lupus erythematosus, Sjogren's syndrome, rheumatoid arthritis, juvenile rheumatoid arthritis, juvenile onset diabetes mellitus (also known as Type I diabetes mellitus), Wegener's granulomatosis, polymyositis, dermatomyositis, inclusion body myositis, multiple endocrine failure, Schmidt's syndrome, autoimmune uveitis, Addison's disease, Graves Disease, Hashimoto's thyroiditis, autoimmune thyroid disease, pernicious anemia, gastric atrophy, chronic hepatitis, lupoid hepatitis, atherosclerosis, presenile dementia, Alzheimer's disease, demyelating diseases, multiple sclerosis, amyotrophic lateral sclerosis, hypoparathyroidism, Dressler's syndrome, myasthenia gravis, Eaton-Lambert syndrome, autoimmune thrombocytopenia, idiopathic thrombocytopenic purpura, hemolytic anemia, pemphigus vulgaris, pemphigus, dermatitis herpetiformis, alopecia, scleroderma, progressive systemic sclerosis, CREST syndrome (calcinosis, Raynaud's phenomenon, esophageal dysmotility, sclerodactyly, and telangtasia), adult onset diabetes mellitus (also known as Type II diabetes mellitus), mixed connective tissue disease, polyarteritis nodosa, systemic necrotizing vasculitis, glomerulonephritis, atopic dermatitis, atopic rhinitis, Goodpasture's syndrome, Chagas' disease, sarcoidosis, rheumatic fever, asthma, anti-phospholipidsyndrome, erythema multiforme, Cushing's syndrome, autoimmune chronic active hepatitis, allergic disease, allergic encephalomyelitis, transfusion reaction, leprosy, malaria, leshmaniasis, trypanosomiasis, Takayasu's arteritis, polymyalgia rheumatica, temporal arteritis, shistosomiasis, gianT-cell arteritis, eczema, lymphomatoid granulomatosis, Kawasaki's disease, dengue fever, encephalomyelitis, endocarditis, endomyocardial fibrosis, endophthalmitis, psoriasis, erythroblastosis fetalis, eosinophilic faciitis, Shulman's syndrome, Felty's syndrome, Fuch's cyclitis, IgA nephropathy, Henoch-Schonlein purpura, graft versus host disease, transplantation rejection, human immunodeficiency virus infection, Epstein-Barr virus infection, mumps, echovirus infection, cardiomyopathy, parvovirus infection, rubella virus infection, anthrax infection, small pox infection, hepatitic C viral infection, tularemia, sepsis, periodic fever syndromes, pyogenic arthritis, Familial Mediterrenan Fever, TNF-receptor associated periodic syndrome (TRAPS), Muckle-Wells syndrome, hyper-IgD syndrome, familial cold urticaria, Hodgkin's and Non-Hodgkin's lymphoma, renal cell carcinoma, or multiple myeloma.

There has also been no known cause for autoimmune diseases such as systemic lupus erythematosus. Systemic lupus erythematosus (SLE) is an autoimmune rheumatic disease characterized by deposition in tissues of autoantibodies and immune complexes leading to tissue injury (Kotzin, 1996). In contrast to autoimmune diseases such as MS and type 1 diabetes mellitus, SLE potentially involves multiple organ systems directly, and its clinical manifestations are diverse and variable (reviewed by Kotzin & O'Dell, 1995). For example, some patients may demonstrate primarily skin rash and joint pain, show spontaneous remissions, and require little medication. At the other end of the spectrum are patients who demonstrate severe and progressive kidney involvement that requires therapy with high doses of steroids and cytotoxic drugs such as cyclophosphamide (Kotzin, 1996).

The serological hallmark of SLE, and the primary diagnostic test available, is elevated serum levels of IgG antibodies to constituents of the cell nucleus, such as double-stranded DNA (dsDNA), single-stranded DNA (ss-DNA), and chromatin. Among these autoantibodies, IgG anti-dsDNA antibodies play a major role in the development of lupus glomerulonephritis (GN) (Hahn & Tsao, 1993; Ohnishi et al., 1994). Glomerulonephritis is a serious condition in which the capillary walls of the kidney's blood purifying glomeruli become thickened by accretions on the epithelial side of glomerular basement membranes. The disease is often chronic and progressive and may lead to eventual renal failure.

The mechanisms by which autoantibodies are induced in these autoimmune diseases remains unclear. As there has been no known cause of SLE, to which diagnosis and/or treatment could be directed, treatment has been directed to suppressing immune responses, for example with macrolide antibiotics, rather than to an underlying cause. (e.g., U.S. Pat. No. 4,843,092).

### D. Combined Therapy

In order to increase the effectiveness of the DC-HIL-toxin therapy, it may be desirable to combine these compositions with another agent effective in the treatment of T-cell inflammatory disorders. The terms “contacted” and “exposed,” when applied to a cell, tissue or organism, are used herein to describe the process by which a therapy and/or other agent are delivered to a target T-cell, tissue or organism or are placed in direct juxtaposition with the target T-cell, tissue or organism. Appropriate “combination” therapies for the various disease states are discussed above, and relevant timing of administration issues are the same as discussed below for combination treatments for T-cell malignancies.

### 4. T-CELL MALIGNANCIES

In one aspect of the present invention, one may utilize toxin conjugates in the treatment of cancer. Any of a variety of T-cell related cancers are contemplated as suitable for treatment with the present invention. The cancer may also be primary, metastatic, multi-drug resistant or recurrent. In one embodiment, the subject will be administered toxin conjugates through a variety of routes including, but not limited to, intravenous, intra-arterial, intra-lymphatic, intralesional, subcutaneous, intraperitoneal, intradermal or intranasal routes. Particular routes include intravenous injection and intralymphatic injection. Repeated or continuous therapy over a period of time (weeks to months) also is contemplated.

A. T-cell Lymphoma

The following is a list of various T-cell lymphomas that may be treated according to the present invention:
Extranodal NK/T-cell lymphoma, nasal type
Enteropathy-type T-cell lymphoma
Hepatosplenic T-cell lymphoma
Blastic NK cell lymphoma
Mycosis fungoides/Sezary syndrome
Primary cutaneous CD30-positive T-cell lymphoproliferative disorders
Primary cutaneous anaplastic large cell lymphoma
Lymphomatoid papulosis
Angioimmunoblastic T-cell lymphoma
Peripheral T-cell lymphoma, unspecified
Anaplastic large cell lymphoma
Adult T-cell lymphoma
Cutaneous T-cell lymphoma

B. T-cell Leukemias

The following is a list of various T-cell leukemias that may be treated according to the present invention:
T-cell prolymphocytic leukemia
T-cell large granular lymphocytic leukemia
Aggressive NK cell leukemia
Adult T-cell leukemia

C. Combined Therapy

In order to increase the effectiveness of the DC-HIL-toxin therapy, it may be desirable to combine these compositions with another agent effective in the treatment of T-cell-related cancers. The terms “contacted” and “exposed,” when applied to a cell, tissue or organism, are used herein to describe the process by which a therapy and/or other agent are delivered to a target T-cell, tissue or organism or are placed in direct juxtaposition with the target T-cell, tissue or organism. Other anti-cancer agents include, but are not limited to, chemotherapeutics, radiotherapeutics or biologicals (anti-CD20 Abs, INFα).

The toxin conjugate treatment may precede, be co-current with and/or follow the other agent(s) by intervals ranging from minutes to weeks. In embodiments where the toxin conjugate treatment and other agent(s) are applied separately to a cell, tissue or organism, one would generally ensure that a significant period of time did not expire between the time of each delivery, such that the toxin conjugate and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) with the toxin conjugate. In other aspects, one or more agents may be administered within of from substantially simultaneously, about 1 minute, about 5 minutes, about 10 minutes, about 20 minutes about 30 minutes, about 45 minutes, about 60 minutes, about 2 hours, about 3 hours, about 4 hours, about 5 hours, about 6 hours, about 7 hours about 8 hours, about 9 hours, about 10 hours, about 11 hours, about 12 hours, about 18 hours, about 24 hours, about 36 hours, about 48 hours, about 3 days, about 4 days, about 5 days, about 6 days, about 7 days, about 8 days, about 9 days, about 10 days, about 11 days, about 12 days, about 13 days, about 14 days, about 21 days, about 4 weeks, about 5 weeks, about 6 weeks, about 7 week or about 8 weeks or more, and any range derivable therein, prior to and/or after administering toxin conjugate.

Various combination regimens of the toxin conjugate treatment and one or more other anti-cancer agents may be employed. Non-limiting examples of such combinations are shown below, wherein a toxin conjugate is “A” and the other anti-cancer agent is “B”:

| | | | | | | | |
|---|---|---|---|---|---|---|---|
| A/B/A | B/A/B | B/B/A | A/A/B | A/B/B | B/A/A | A/B/B/B | B/A/B/B |

| | | | | | |
|---|---|---|---|---|---|
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Other combinations are contemplated. Again, to achieve cancer cell inhibition, both agents are delivered to a cell in a combined amount effective to achieve the desired inhibition, which may include cell stasis or cell death.

Administration of the toxin conjugate to a cell, tissue or organism may follow general protocols for the administration of pharmaceuticals. It is expected that the treatment cycles would be repeated as necessary. In particular embodiments, it is contemplated that various additional agents may be applied in any combination with the present invention.

Radiation agents and factors include radiation and waves that induce DNA damage such as, γ-irradiation, X-rays, UV-irradiation, microwaves, electronic emissions, and the like. A variety of chemical compounds, also described as “chemotherapeutic agents,” function to induce DNA damage, all of which are intended to be of use in the combined treatment methods disclosed herein. Chemotherapeutic agents contemplated to be of use, include, e.g., doxorubicin, verapamil, podophyllotoxin, adriamycin, 5-fluorouracil (5FU), etoposide (VP-16), camptothecin, actinomycin-D, mitomycin C, cisplatin (CDDP) and even hydrogen peroxide. Biologicals include cytokines, interferons, and antibodies.

### 5. PHARMACEUTICAL FORMULATIONS

Pharmaceutical formulations of the present invention comprise an effective amount of a toxin conjugate dissolved or dispersed in a pharmaceutically acceptable carrier. The phrases “pharmaceutical or pharmacologically acceptable” refer to compositions that do not produce an adverse, allergic or other untoward reaction when administered to an animal, such as, for example, a human, as appropriate. The preparation of such pharmaceutical compositions are known to those of skill in the art in light of the present disclosure, as exemplified by Remington's Pharmaceutical Sciences, 18^{th} Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art. Except insofar as any conventional carrier is incompatible with the active ingredient, its use in the therapeutic or pharmaceutical compositions is contemplated.

The pharmaceuticals of the present invention may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The pharmaceuticals may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc.), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain embodiments, the compositions are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

### 6. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Mice and cell culture. Female BALB/c and C57BL/6 (5-8 weeks old) mice were obtained from the Animal Breeding Center at The University of Texas Southwestern Medical Center (Dallas, Tex.). BALB/cTac-TgN(DO11.10)-Rag2tm1 mice (Hsieh et al., 1993) were purchased from Taconic (Hudson, N.Y.). Following National Institutes of Health guidelines, these animals were housed and cared for in the pathogen-free facility, and all animal studies were approved by the Institutional Animal Care Use Center of the same institution.

Production of DC-HIL-Fc protein. The Fc-fusion proteins (DC-HIL-Fc, its mutants, and Fc alone) were produced in COS-1 cells and purified as described previously (Shikano et al., 2001). Isolation of T-cells and binding of DC-HIL. Following manufacturer's recommendations, CD3⁺, CD4⁺, and CD8⁺ T-cells were purified from spleen cells of BALB/c mice using pan-T-cell, CD4⁺, and CD8⁺ T-cell isolation kits (Miltenyi Biotec, Auburn, Calif.), respectively.

Splenic CD3⁺ T-cells (1×10⁶) were activated by immobilized anti-CD3 Ab (1 or 3 μg/mL) or concanavalin A (10 μg/mL; Sigma, St Louis, Mo.) for 3 days. Freshly isolated and activated CD3⁺ T-cells were then treated with 5 μg/mL Fc blocker (BD Pharmingen, San Diego, Calif.) on ice for 30 minutes to block Fc-binding activity of Fc receptors on T-cells and incubated with 5 μg/mL PE-anti-CD3 Ab (BD Pharmingen) and 10 μg/mL DC-HIL-Fc or control human IgG (hIgG) plus 5 μg/mL FITC-anti-human IgG (both from Jackson ImmunoResearch, West Grove, Pa.). In some experiments, activated and Fc-blocked CD3⁺ T-cells were doubly-stained with 5 μg/mL FITC-labeled anti-CD4 or anti-CD8 Ab (BD Pharmingen) and Fc proteins/PE-anti-human IgG (BD Pharmingen). The treated cells were also stained with anti-CD69 Ab or isotypic control IgG to evaluate activation levels. After staining, binding of Fc proteins to T-cells and expression of marker molecules were analyzed by fluorescence-activated cell sorting (FACS).

T-cell proliferation and IL-2 assay. Purified CD4⁺ or CD8⁺ T-cells (2×10⁵/well) were cultured for 2 days in enzyme-linked immunosorbent assay (ELISA) wells (in triplicate) precoated with indicated doses of anti-CD3 Ab and Fc proteins or anti-CD28 mAb (BD Pharmingen). After pulsing with ³H-thymidine (1 μCi/well [0.037 MBq/well]) for 20 to 22 hours, cells were collected and evaluated for ³H radioactivity. Culture supernatant was used to measure IL-2 production using the mouse IL-2 ELISA kit (BD Pharmingen). To examine the effects of DC-HIL on reactivation of previously activated T-cells, spleen cells (1×10⁶/mL) isolated from Tac-TgN(DO11.10)-Rag2tm1 mice were cultured for 3 days in the presence of the ovalbumin OVA323-339 peptide (1 μg/mL).24 After purifying CD4⁺ T-cells from the culture, cells (1×10⁶/mL) were cultured for another day without stimuli and then subjected to T-cell proliferation and IL-2 assays.

Cell-cycle analyses. Cell cycles of CD4⁺ T-cells treated with anti-CD3 Ab and Fc protein were examined using carboxy-fluorescein diacetate succinimidyl ester (CFSE; Molecular Probes, Eugene, Oreg.). Purified CD4⁺ T-cells (1×10⁶) were labeled by 1 μM CFSE/DPBS for 15 minutes at 37° C. After another 30 minutes of incubation in culture medium, labeled T-cells were cultured in ELISA wells coated with anti-CD3 Ab (0.3 μg/mL) and control IgG or DC-HIL-Fc (5 μg/mL). At different time points, cells were harvested to examine asynchronous cell division by FACS. Cell cycles of treated T-cells were also analyzed using FITC-BrdU flow kit (BD Pharmingen), following the manufacturer's recommendations. BrdU incorporation and DNA content on a per-cell basis were analyzed by FACS and presented as dot plots.

MLR. BALB/c T-cells and C57BL/6 spleen cells served as responders and stimulators, respectively. C57BL/6 spleen cells (5×10⁴) were y-irradiated (2000 Gy) and mixed with CD4⁺ T-cells (2×10⁵) purified from BALB/c spleen in 96-microwell plates. Fc fusion protein or control hIgG was added to the mixed leukocyte reaction (MLR) culture and incubated for varying periods. After ³H-thymidine pulsing for 20 hours, cells were harvested and the cell-incorporated ³H radioactivity was measured. T-cell proliferation was expressed as radioactivity left after subtracting background counts per minute (cpm; ³H cpm of control culture in which y-irradiated responders and stimulators were mixed) from experimental cpm.

In vitro antigen presentation by DCs. BM cells were prepared from the femurs of BALB/c mice and cultured with 10 ng/mL of granulocyte-macrophage colony-stimulating factor (GM-CSF; PeproTech, Rocky Hill, N.J.).25 After culturing for 6 days, DCs were harvested, seeded on 96-well plates at a density of 5×10⁵ cells/well, and cultured with OVA323-339 peptide (2 μg/mL)24 synthesized by the Protein Chemistry Technology Center, UT Southwestern. After 6 hours of antigen pulsing, DC cultures (5×10⁴ cells) were added with CD4⁺ T-cells (1×10⁵/well) purified from the spleens of BALB/cTac-TgN(DO11.10)-Rag2tm1 mice (Hsieh et al., 1993). After coculture (2 days), cells and supernatant were harvested: cells were stained with FITC-anti-CD4 and PE-anti-CD69 Ab and determined by FACS for frequency of CD69⁺ cells in CD4⁺ T-cells. The supernatant was assayed by ELISA for IL-2 production.

Knockdown of DC-HIL expression. A total of 2 different DC-HIL-targeted siRNAs (21 nucleotides long) were synthesized and purified by Qiagen (Valencia, Calif.): DC-HIL siRNA no. 3, sense 5′-r(AACUUGUCUGAUGAGAUCU)dTdT-3′ (SEQ ID NO:6) and antisense 5′-r(AGAUCUCAUCAGACAAGUU)dTdT-3′ (SEQ ID NO:7); DC-HIL siRNA no. 10, sense 5′-r(GCGUACAAGCCAAUAGGAA)dTdT-3′ (SEQ ID NO:8) and antisense, 5′-r(UUCCUAUUGGCUUGUACGC)dTdT-3′ (SEQ ID NO:9). Shuffled sequences of these 2 siRNAs were used as controls. A mixture of siRNA no. 3 and no. 10 (each 1.5 μg) or of the shuffled siRNAs was treated with 5 μL of Gene Silencer (Genlantis, San Diego, Calif.) in the serum-free RPMI for 20 min at room temperature and then added to BM-DCs (2×10⁶ cells). After culturing for 4 hours at 37° C., 20% FCS-RPMI was added to the culture and allowed to incubate for another 24 hours. These transfected DCs were harvested and examined by Western blotting for protein expression of DC-HIL or mixed with OVA-specific T-cells as described in “In vitro antigen presentation by HSCs.”

CH assays. BALB/c mice (n=5) were sensitized for contact hypersensitivity (CH) on day 0 by painting 2% oxazolone (Ox; Sigma) in acetone-olive oil (4:1 in volume) on shaved abdominal skin (sensitization) (Cruz et al., 1988). Mice were challenged on day 6 by painting 1% Ox and solvent control onto right and left ears, respectively (elicitation). Thereafter, CH was assessed daily through day 12 by measuring ear thickness and calculating changes in ear swelling (thickness of right ear minus thickness of control left ear) (Cruz and Bergstresser, 1988). Different panels of mice were injected intraperitoneally with DC-HIL-Fc or the control hIgG (10 mg/kg each) or DPBS on days −1, 1, and 3 (before and after sensitization) or on days 5, 7, and 9 (before and after elicitation). The Student t test was used to determine statistically significant differences in ear-swelling responses.

Histologic examination of skin and phenotyping of LN cells. After painting Ox on ears of Ox-sensitized mice treated with Fc protein (2 days), ear skin and draining lymph nodes (LNs) were procured. Ear skin was embedded in paraffin, thin-sectioned, and stained with hematoxylineosin (Sigma). Histologic examination was carried out under light microscopy using an Olympus BH2 microscope (Olympus, Center Valley, Pa.) at a magnification of ×10. In independent experiments, unsensitized mice were treated similarly and their draining LNs excised 2 days after ear challenge. LN cells were counted and examined for spontaneous proliferation and frequency of CD69⁺ cells.

For proliferation, LN cells (4×10⁵/well) from untreated or treated mice were cultured without stimulation for 3 days and pulsed with ³H-thymidine (1 μCi/well [0.037 MBq/well]) for 20 hours. For CD69 expression, LN cells (5×10⁵) were stained with FITC-anti-CD69 mAb (eBioscience, San Diego, Calif.) or FITC-isotypic control hamster IgG (BD Pharmingen) (2.5 μg/mL each) in the presence or absence of Ab directed at T-cell or B-cell surface markers (CD4, CD8, and B220; 2.5 μg/mL each) and examined by FACS for surface expression of CD69 in each leukocyte subpopulation.

Generation of mutants DC-HIL-Fc carrying the RAA mutant (replacement of RGD sequence with RAA) was generated as before (Shikano et al., 2001). PRR and PKD mutants (lacking a region between amino acids 301 to 334 and 230 to 355, respectively) were produced by polymerase chain reaction (PCR)-based mutagenesis. Resulting nucleotides coding extracellular domains of DC-HIL mutants were inserted in-frame to the coding sequence of the human IgG1 Fc in pSecTagA plasmid (Invitrogen, Carlsbad, Calif.) using 3 restriction enzyme sites (from the 5′ end, HindIII, EcoRI, and XbaI). The mutant DC-HIL-Fc proteins were produced as described previously (Shikano et al., 2001). The yield for each mutant was very similar to the wild-type and all preparations showed a single band reactive to anti-human IgG Ab.

### Example 2

### Results

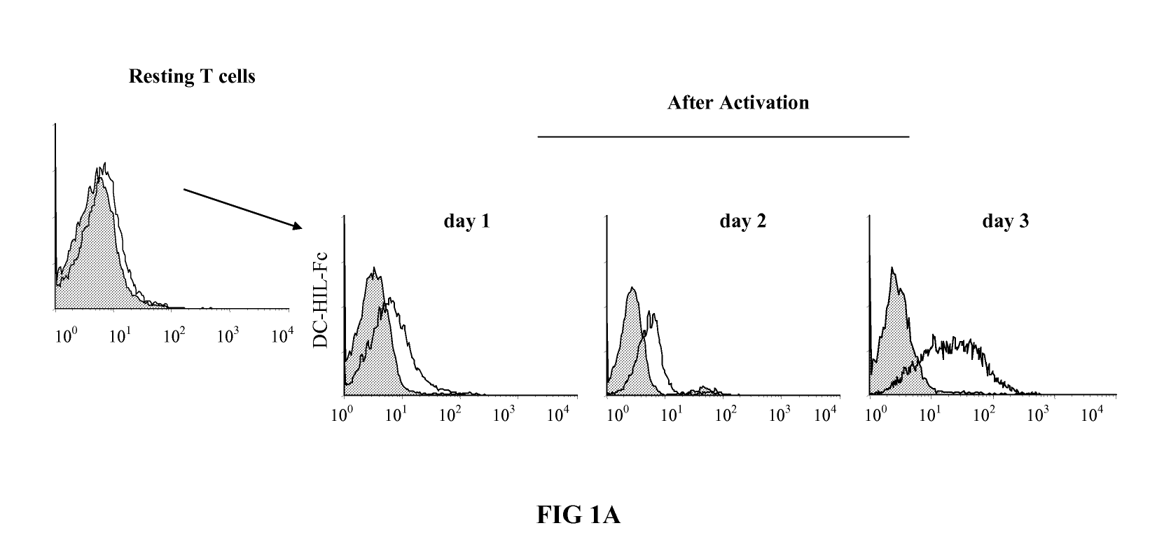

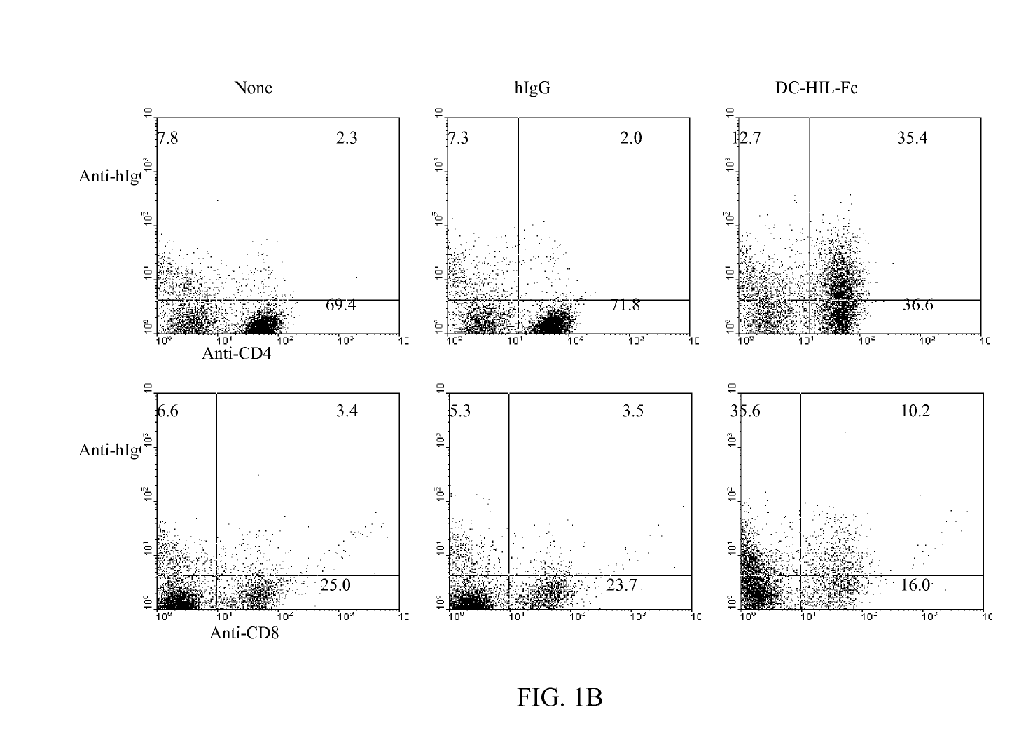

Activated T-cells express ligands of DC-HIL. To study the function of DC-HIL, the inventors created soluble DC-HIL receptors (DC-HIL-Fc) consisting of the extracellular domain fused with the Fc portion of human IgG1 (hIgG) ((Shikano et al., 2001), and used FACS analysis to examine binding to T-cells. DC-HIL-Fc did not bind to T-cells freshly isolated from spleen of naive mice, but did so after the T-cells were activated by concanavalin A (FIG. 1A) or by immobilized anti-CD3 Ab (FIG. 1B). Binding was noted as early as a day after stimulation and lasted for at least 3 days. The inventors also examined binding to T-cell subsets (FIG. 1B) and observed that activated CD4⁺ and CD8⁺ T-cells were bound at frequencies of 35.4% and 10.2%, respectively. These results indicated that T-cells express putative ligands of DC-HIL (DC-HIL-L) after activation. Involvement of Fc receptors in binding was excluded by failure of Fc blocker to inhibit binding of DC-HIL-Fc to T-cells and inability of recombinant Fc alone to bind T-cells (data not shown).

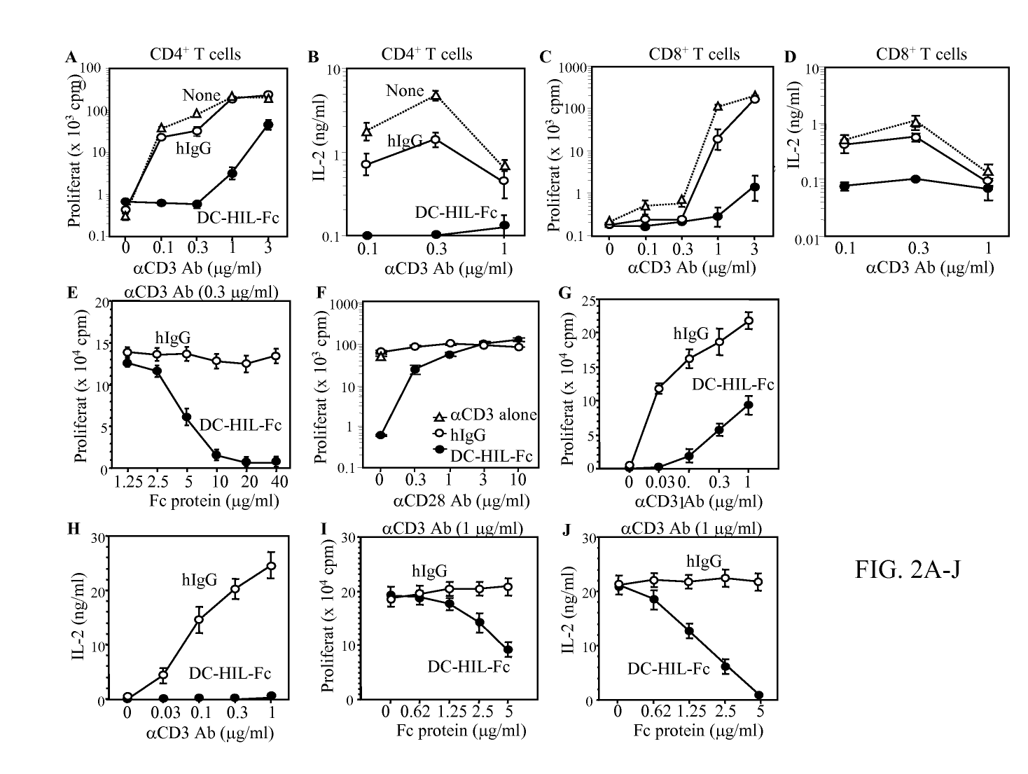

Immobilized DC-HIL inhibits T-cell activation triggered via the TCR. The inventors next used immobilized anti-CD3 Ab as a surrogate stimulator of TCR-dependent T-cell activation. CD4⁺ T-cells were cultured in microculture wells precoated with increasing concentrations of anti-CD3 Ab and a constant concentration of DC-HIL-Fc or control hIgG. T-cell activation was assessed by proliferative capacity measured by ³H-thymidine incorporation. Treatment with immobilized anti-CD3 Ab led to activation of CD4⁺ T-cells in a dose-dependent manner (FIG. 2A). Coimmobilization of DCHIL-Fc with anti-CD3 Ab attenuated CD4⁺ T-cell activation at all doses of anti-CD3 Ab tested; inhibition was most marked at suboptimal doses (0.1 and 0.3 μg/mL) of anti-CD3 Ab, and was counteracted by increasing doses of anti-CD3 Ab (1 and 3 μg/mL).

By contrast, coimmobilization of control hIgG with anti-CD3 Ab had almost no effect on CD4⁺ T-cell activation. An irrelevant Fc fusion protein dectin-2-Fc (Sato et al., 2006), a C-type lectin receptor, had little to no effect on T-cell activation. The ability of DC-HIL to inhibit T-cell activation was also documented by little to no IL-2 secreted by T-cells treated with DC-HIL-Fc (FIG. 2B). Similar outcomes were observed for CD8⁺ T-cells, although the degree of DC-HIL-induced inhibition was less than for CD4⁺ T-cells (FIGS. 2C-D). These results indicate that binding of immobilized DCHIL-Fc to its putative ligand on T-cells inhibits TCR-dependent proliferation and IL-2 production. Indeed, addition of exogenous IL-2 rescued inhibition induced by DC-HIL (data not shown). The inventors next titrated the inhibitory capacity of DC-HIL against the stimulatory ability of anti-CD3 Ab by keeping the dose of the latter constant (0.3 μg/mL) while adding the former in incremental doses (FIG. 2E). Doses of DC-HIL-Fc greater than 5 μg/mL were required to inhibit T-cell activation.

Because ligation of CD28 on T-cells amplifies TCR-mediated T-cell activation (Linsley and Ledbetter, 1993), the inventors questioned whether CD28 co-stimulation can rescue DC-HIL-induced inhibition (FIG. 2F). Anti-CD28 mAb (in increasing doses) was immobilized on microwells precoated with DC-HIL-Fc or hIgG (10 μg/mL each) and anti-CD3 Ab (0.3 μg/mL). Purified CD4⁺ T-cells were cultured in coated wells, and activation status was determined by ³H-thymidine incorporation. In the absence of anti-CD28 mAb, DC-HIL-Fc again markedly inhibited T-cell activation (FIG. 2F). However, such inhibition was overcome by addition of anti-CD28 mAb in a dose-dependent manner, with 3 μg/mL anti-CD28 mAb completely rescuing DC-HIL-Fc-induced inhibition.

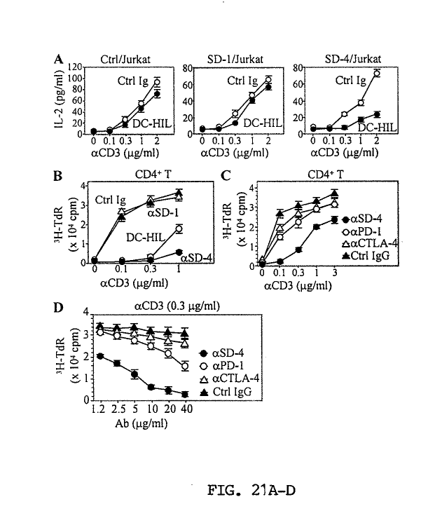

The inventors also examined whether DC-HIL exerts inhibitory effects on previously activated T-cells (FIGS. 2G-J). Spleen cells isolated from BALB/cTac-TgN(DO11.10)-Rag2tm1 mice (Hsieh et al., 1993) bearing a transgene encoding TCR specific for the ovalbumin peptide (OVA323-339) (Inaba et al., 1992) were activated by adding the OVA peptide to the culture. Activated CD4⁺ T-cells were isolated and reactivated with immobilized anti-CD3 Ab and DC-HIL-Fc or control Ig. DC-HIL markedly inhibited T-cell proliferation (FIG. 2G) in response to anti-CD3Ab and abrogated IL-2 production (FIG. 2H). The inventors also titrated the inhibitory capacity of DC-HIL against the reactivation, as described previously (FIGS. 2I-J). Similar to effects on naive T-cells (FIG. 2E), DC-HIL-Fc strongly inhibited T-cell proliferation at 5 μg/mL (FIG. 21), and reduction of IL-2 production was even greater (FIG. 2J). These results indicate that the putative T-cell ligand for DC-HIL mediates a potent signal that can inhibit activation of primary as well as secondary T-cell responses.

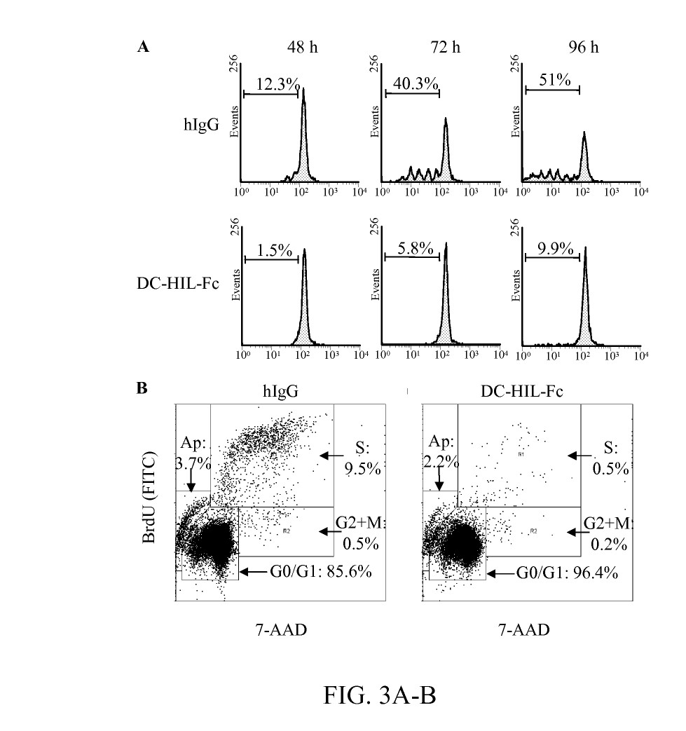

Binding of DC-HIL to T-cells induces cell-cycle arrest. Since PD-1-mediated signals prevented anti-CD3 Ab-treated T-cells from entering the cell cycle (Latchman et al., 2001; Freeman et al., 2000), the inventors questioned whether inhibition of T-cell activation by DC-HIL-Fc is similarly achieved. CD4⁺ T-cells were cultured in wells coated with anti-CD3 Ab plus DC-HIL-Fc or control hIgG; at different time points thereafter, the T-cells were assayed for asynchronous cell division using CFSE labeling and FACS analysis (FIG. 3A). For anti-CD3 Ab and hIgG-treated cells, the number of cell divisions increased 6-fold and the frequency of divided cells increased up to 51%. DC-HILFc-treated T-cells also divided several times, but at a lesser frequency (FIG. 3A). To more precisely analyze cell-cycle effects, T-cells at 48 hours after activation were labeled with 7-AAD (to stain chromosomal DNA in all cells) and immunofluorescent BrdU (to stain proliferating cells) (FIG. 3B). Based on a ratio of 7-AAD-staining to BrdU-staining intensities, T-cells treated with anti-CD3 Ab and control hIgG sorted into S phase (9.5%), G2/M phase (0.5%), G0/G1 phase (85.6%), and apoptotic cells (3.7%). For cells treated with anti-CD3 Ab and DC-HIL-Fc, similar portions of cells sorted into G2/M phase (0.2%), G0/G1 phase (96.4%), and apoptotic cells (2.2%), but markedly fewer were sorted into S phase (0.5%). These results indicate that signaling through the putative DC-HIL-L leads to cell-cycle arrest (rather than apoptosis).

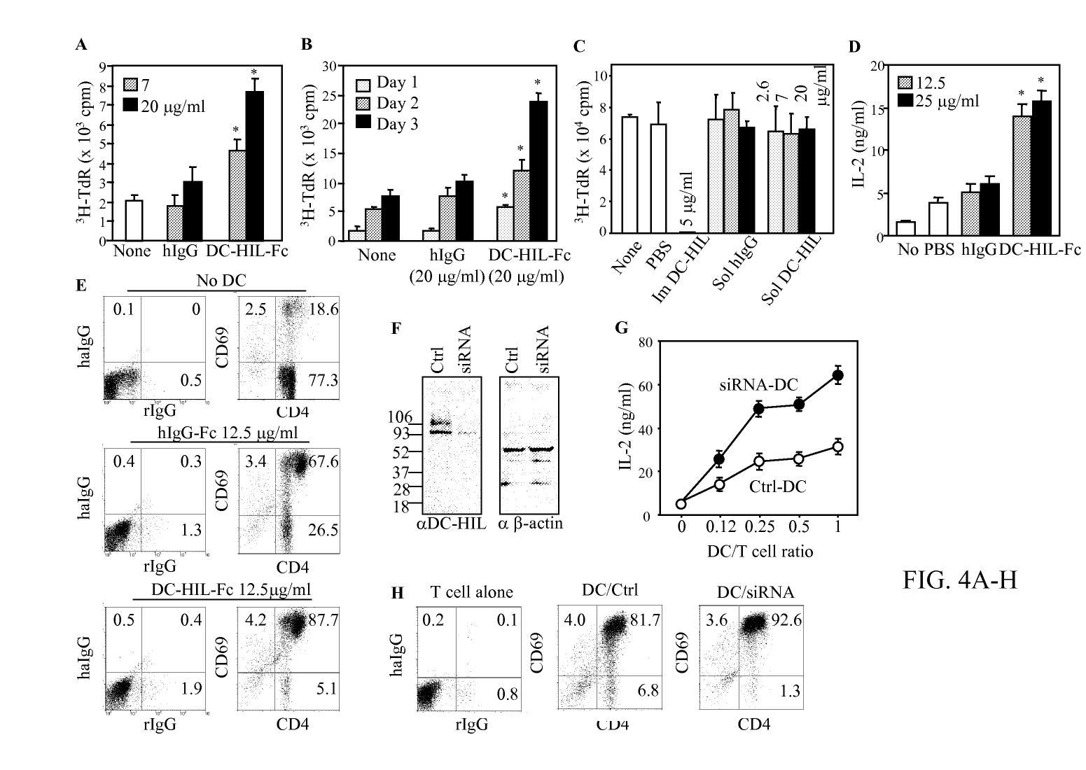

Soluble DC-HIL enhances T-cell activation triggered by APCs. The inventors next examined the effect of soluble DC-HIL-Fc on the MLR, in which alloreactive T-cells are activated by APCs. In this MLR, spleen cells from C57BL/6 mice served as stimulators, CD4⁺ T-cells from BALB/c mice served as responders, and the proliferative capacity of responder cells was measured by ³H-thymidine incorporation (FIGS. 4A-B). To the inventors' surprise, the addition of DC-HIL-Fc (but not hIgG) enhanced the MLR in a dose- and time-dependent manner. Control hIgG or an irrelevant Fc fusion protein dectin-2-Fc had no effect on MLR.

Because the enhancing effect of soluble DC-HIL on the MLR contrasted with the inhibitory effect of immobilized DC-HIL-Fc on anti-CD3 Ab-induced T-cell proliferation (FIGS. 2A-J), the inventors compared effects of soluble versus immobilized DC-HIL-Fc in parallel, using in vitro T-cell proliferation assays in which immobilized anti-CD3 Ab (rather than APCs) served as the primary stimulator (FIG. 4C). Immobilized DC-HIL-Fc (5 μg/mL) abrogated T-cell activation, whereas soluble DC-HIL-Fc (final concentration of 20 μg/mL) had no effect on activation of purified T-cells. Taken together, these results indicate that soluble DC-HIL-Fc is capable of binding to T-cells, but fails to transduce signals in T-cells; more than likely, it interferes with binding of DC-HIL (on APCs) to DC-HIL-L (on T-cells), thereby neutralizing the inhibitory function of endogenous DC-HIL, leading to an enhanced MLR.

To more rigorously examine the effects of soluble DC-HIL-Fc on T-cell activation, the inventors used an in vitro antigen presentation assay in which BM-DCs were pulsed with OVA peptide (OVA323-339) (Demotz et al., 1993) and allowed to activate CD4⁺ T-cells prepared from unprimed BALB/cTac-TgN(DO11.10)-Rag2tm1 mice. Different doses of DCHIL-Fc or control hIgG were added to the assay, and T-cell activation was measured by IL-2 production and frequency of CD69⁺/CD4⁺ cells (FIGS. 4D-E). DC-HIL-Fc augmented T-cell activation in a dose-dependent manner (up to 3-fold increase in IL-2 production); control hIgG had very little effect (binding of hIgG to DCs was very weak). DC-HIL-Fc-treated T-cells showed an increased frequency (88%) of activated (CD69⁺) cells (versus 68% for controls). Having shown previously that both immature and mature DCs express DC-HIL on the surface, the inventors interpreted these results to mean that soluble DC-HIL blocked endogenous DC-HIL function, leading to augmented T-cell responses.

DCs with knockdown DC-HIL display enhanced immunostimulatory capacity. To evaluate the function of DC-associated DC-HIL, the inventors prepared DCs with genetically modified (knockdown) expression of DC-HIL and examined immunostimulatory capacity. Transfection of DC-HIL siRNA inhibited most of the DC-HIL protein expression in DCs when compared with DCs with control siRNA (FIG. 4F). Increasing numbers of these transfected DCs were pulsed with OVA peptide and then allowed to activate a constant number of CD4⁺ T-cells purified from Tac-TgN(DO11.10)-Rag2tm1 mice. T-cell activation was assayed by IL-2 production (FIG. 4G). At every dose tested, DC-HIL siRNA-transfected DCs were more potent activators of OVA-specific T-cells, compared with control siRNA DCs. Enhanced activation was also affirmed by a higher frequency of CD69⁺T-cells (92.6% vs 81.7%) in coculture (FIG. 4H).

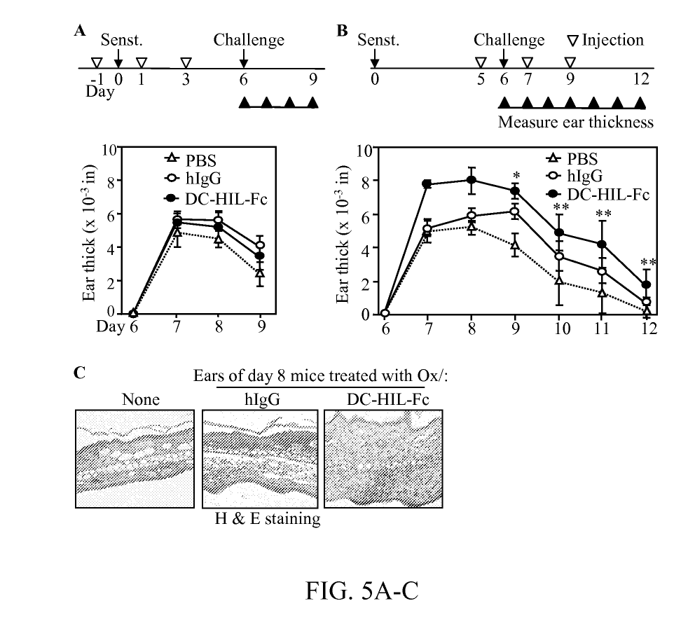

In vivo injection of soluble DC-HIL augments CH responses. To ascertain the biological significance of the DC-HIL/DC-HIL-L pathway, the inventors examined the effects of soluble DC-HIL on CH, an experimental model of delayed-type, T-cell-mediated skin inflammation. BALB/c mice were sensitized by topical application of the hapten Ox at a dose of 2% on abdominal skin (day 0) and then challenged/elicited by painting ears with 1% Ox (day 6) (FIGS. 5A-C). Mice in different panels were injected intraperitoneally with DC-HIL-Fc, hIgG, or PBS every other day on 3 occasions either starting a day before sensitization (FIG. 5A) or a day before elicitation (FIG. 5B). Ear swelling was measured and change in ear thickness was calculated. Ear swelling in mice treated with DC-HIL-Fc just before and after hapten sensitization was no different from that of mice injected with control hIgG (FIG. 5A).

By contrast, mice injected with DC-HIL-Fc just before and after hapten elicitation displayed significantly greater ear swelling that persisted longer (up to 6 days after elicitation), compared to those of control mice (FIG. 5B). Histologic analysis of Ox-painted ear skin revealed a marked increase in skin thickness and number of skin-infiltrating leukocytes in mice treated with DC-HIL-Fc, but not with control hIgG (FIG. 5C).

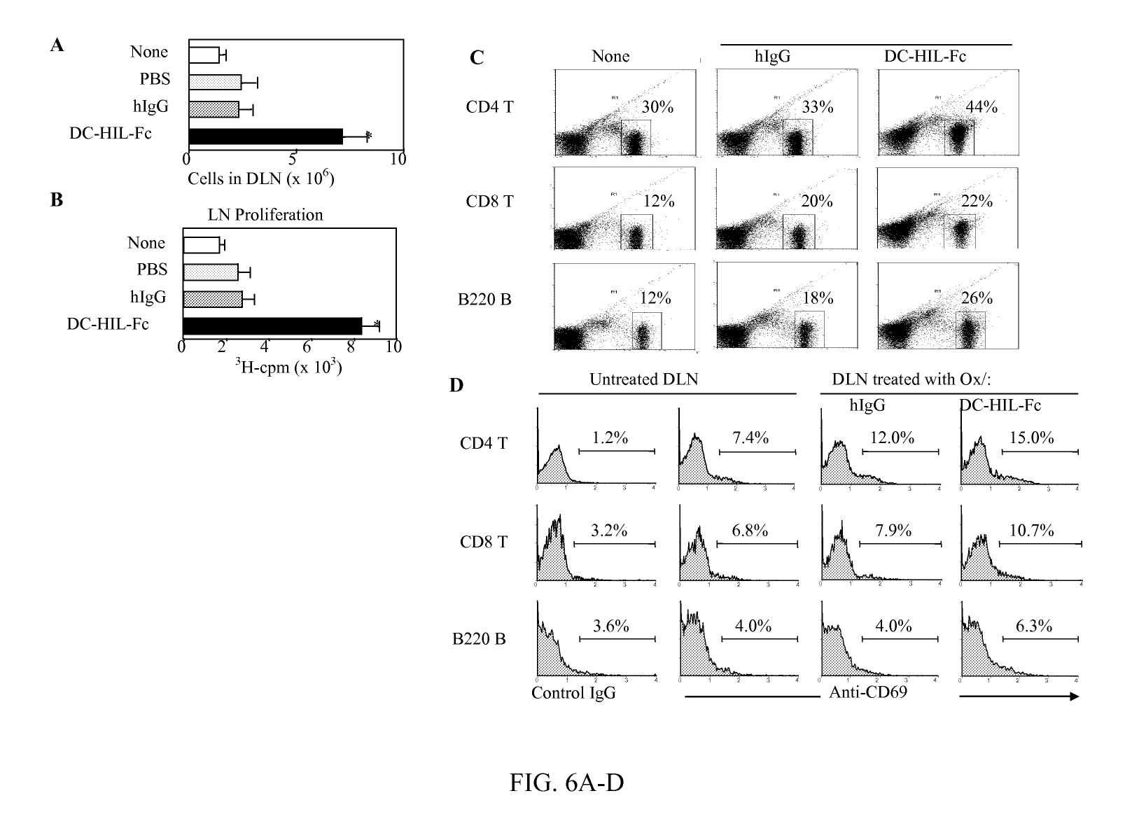

To determine the activation status of T-cells in Ox-sensitized mice injected with DC-HIL-Fc, the inventors compared draining LNs and LN cells of DC-HIL-Fc-treated versus control mice 2 days after Ox challenge, which was the time of greatest ear swelling. LNs of DC-HIL-treated mice were 3 times larger than hIgG-treated mice, contained 3 times the number of cells (FIG. 6A), and displayed 3-fold greater T-cell proliferation in the absence of stimuli (FIG. 6B). The inventors also compared the frequency of CD4⁺, CD8⁺, or B220⁺ LN cells (FIG. 6C). Draining LN cells of DC-HIL-treated mice had greater portions of CD4⁺ T-cells (44% vs 33%) and of B cells (25% vs 18%) compared with those of hIgG-treated mice. Finally, the inventors examined the frequency of CD69⁺ (activated) cells (FIG. 6D). DC-HIL treatment was associated with an increase in the activation phenotype for each of the 3 leukocyte subpopulations examined: 15% versus 12% for CD4⁺ T-cells, 10.7% versus 7.9% for CD8⁺ T-cells, and 6.3% versus 4% for B cells. These findings constitute in vivo support for previous in vitro observations that (i) DC-HIL-L are expressed on activated (but not resting) T-cells, (ii) DC-HIL is a negative regulator of T-cell activation, and (iii) soluble DC-HIL enhances T-cell-mediated responses.

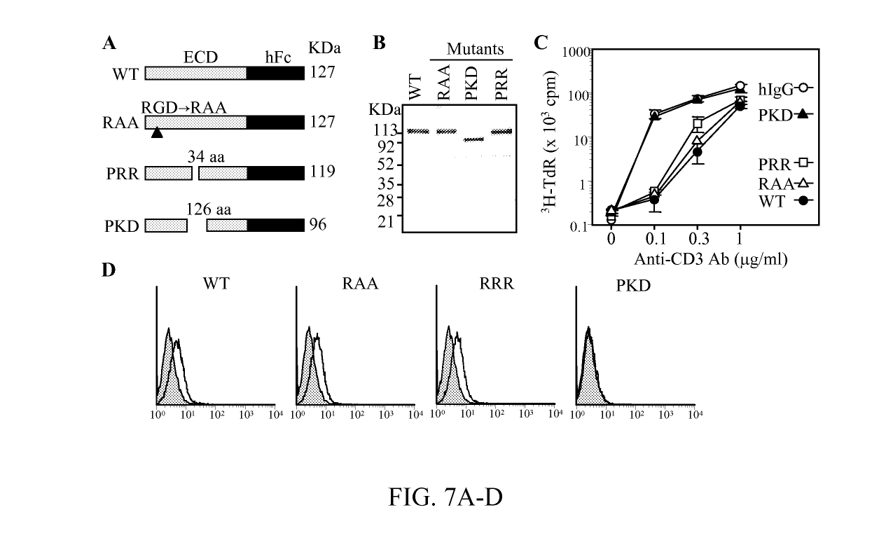

PKD domain is required for the inhibitory function of DC-HIL on T-cell activation. The inventors reported previously that the extracellular domain of DC-HIL contains an RGD motif required for integrin-mediated cell adhesion; an Ig-like polycystic kidney disease (PKD) domain (Bycroft et al., 1999), and a proline-rich region (PRR) involved in protein-protein interactions (Kay et al., 2000). To determine whether some or all are required for the inhibitory function of DC-HIL on T-cell activation, the inventors created extracellular domains of mutant DC-HIL lacking PKD or PRR, or containing RAA (instead of RGD), and fused these mutants to the IgG-Fc (FIG. 7A). Purity of the mutants was quite high (similar to the wild-type) as judged by SDS-PAGE/Coomassie blue staining (FIG. 7B). The inventors then examined the capacity of mutants to inhibit T-cell activation by titrating a given dose of mutant (or wild-type) DC-HIL-Fc to increasing doses of anti-CD3 Ab (FIG. 7C). The inhibitory activity of wild-type DC-HIL-Fc (5 μg/mL) decreased progressively with increasing doses of anti-CD3 Ab. The titration curve of the RAA mutant was similar to the wild-type. The PRR-deficient mutant showed only minimally reduced inhibitory activity at a dose of 0.3 μg/mL anti-CD3 Ab. By contrast, the PKD-deficient mutant almost completely lost inhibitory activity at each dose of anti-CD3 Ab tested. The inventors also compared mutants versus wild-type with respect to T-cell-binding capacity (FIG. 7D).

RAA and PRR mutants bound to T-cells as efficiently as the wild-type, whereas the PKD mutant failed to bind to T-cells (correlating with results of inhibitory function). These findings indicate that the Ig-like PKD domain is required for binding of DC-HIL to T-cells and for its inhibitory function. By contrast, neither proline-mediated interaction nor RGD-dependenT-cell adhesion appears necessary for DC-HIL's inhibitory function.

### Example 3

### Discussion

Subtractive cDNA cloning of mouse XS52 DCs minus J774 macrophages (Shikano et al., 2001) led to the inventors' discovery of DC-HIL, also known as human nmb glycoprotein or gpnmb (Anderson et al., 2002; Weterman et al., 1995) and rat osteoactivin (Safadi et al., 2001). The extracellular domain of DC-HIL contains a putative heparin-binding site (Cardin and Weintraub, 1989), many N-glycosylation sites, an RGD cell-adhesion motif (Ruoslahti, 1996), a PRR (involved in O-glycosylation (Wilson et al., 1991) and/or protein-protein interactions (Kay et al., 2000)), and an Ig-like PKD domain conserved among 14 repeats in the extracellular region of the PKD-susceptible gene product, product, polycystin-1 (Bycroft et al., 1999; Ponting et al., 1999). Moreover, the inventors showed that DC-HIL is highly N-glycosylated, that it recognizes heparan sulfate (especially on small-vessel endothelial cells [SVECs]) (Shikano et al., 2001) and that its RGD motif is responsible for integrin-mediated cell adhesion (Shikano et al., 2001). The have also uncovered a new function for DC-HIL, as a potent inhibitor of TCR-induced T-cell activation for both primary and secondary responses.

Compared with known pairs of inhibitory regulators of T-cell activation, DC-HIL and its putative ligand (DC-HIL-L) best resemble of PD-L1/PDL2 and its ligand, PD-1. For example, unlike BTLA8 or Tim-3 (Sabatos et al., 2003; Sanchez-Fueyo et al., 2003) whose expressions are restricted strictly to leukocytes, DC-HIL or PD-L1/PD-L2 expression can be induced in non-leukocytes by proinflammatory stimuli like IFN-γ (Freeman et al., 2000). Moreover, in contrast to B7-1/B7-2, whose T-cell ligand CD28 is present on resting T-cells, the T-cell ligand of DC-HIL or of PD-L1/PD-L2 is not expressed constitutively, requiring activation of the T-cells for expression. Engagement of DC-HIL or of PD-L1/PD-L2 with their respective T-cell ligand attenuates T-cell activation, suppresses IL-2 secretion, and arrests T-cell proliferation, all of which can be rescued by costimulation of CD28. By contrast, interference with binding of DC-HIL to DC-HIL-L or of PD-L1/PD-L2 to PD-139 leads to enhanced T-cell responses in MLR and Ag-specific reactions in vitro and in CH in vivo. This antagonism by soluble DC-HIL is not unusual, since other Fc-tagged recombinant proteins have been used to block the endogenous functions of CD200, TREM-1, PD-1, DIgR2, and other receptors, respectively (Brown et al., 2003; Gorczynski et al., 2004; Gibot et al., 2004; Shi et al., 2006).

As cited previously, all known pairs of T-cell inhibitory regulators on APCs and their ligands on T-cells (including CD80 and CD86, which bind to CTLA-4 (Krummel and Allison, 1995; Tsushima et al., 2003); and PD-L1/PD-L2, which bind to PD-1 (Latchman et al., 2001; Dong et al., 1999); possess Ig domains that allow their categorization as members of the B7 receptor superfamily. Indeed, the Ig domains are responsible for ligation of each pair. DC-HIL differs from these inhibitors in not possessing an Ig domain typical of the B7 receptor family, instead containing a PKD domain that can fold into an Ig-like tertiary structure critical to its binding and inhibitory functions.

An important task ahead is the identification of the DC-HIL-L on activated T-cells. Given the previous finding that DC-HIL recognizes heparin sulfate on endothelial cells (Shikano et al., 2001), the inventors speculate that heparan sulfate is involved in the binding of DC-HIL to DC-HIL-L. However, heparan sulfate alone is not likely to be the complete ligand since the PKD-deficient DC-HIL mutant the inventors tested bound heparan sulfate but not activated T-cells. Rather, the inventors hypothesize the putative ligand to bear heparan sulfate plus a peptide with affinity for the PKD domain.

### Example 4

### Materials and Methods

Mice. Female BALB/c and C57BL/6 (5-8 wk old) mice were purchased from HarLan (Indianapolis, Ind.) and BALB/cTac-TgN(DO11.10)-Rag2^{tm1} (or DO11.10) mice from Taconic (Hudson, N.Y.). Following National Institutes of Health guidelines, mice were housed and cared for in the pathogen-free facility, and subjected to experimental procedures approved by Institutional Animal Care Use Center at The University of Texas Southwestern Medical Center, Dallas, Tex.

Construction of plasmid vectors. The previously constructed pSTB-DC-HIL-Fc, which encodes the extracellular domain of DC-HIL fused to the Fc portion of human IgG₁. A plasmid pSTB-SD4-Fc was constructed by replacing the extracellular domain with that of SD-4 obtained by RT-PCR. The V5 epitope sequence was inserted just after the leader sequence of the SD-4 encoding sequence (V5-SD4) and introduced into a lentiviral vector plasmid, pHR-SIN-CSGW (GFP)-Ub-Em (gift from Y. Ikeda, Mayo Clinic, Rochester, Minn.). This recombinant lentivirus co-expresses Emerald GFP and V5-SD4. Infectious particles were prepared and their titration performed according to established protocols.

Generation of DO11.10 expressing V5-SD4. AT-cell hybridoma DO11.10 line (provided by J. Kappler and P. Marrack, National Jewish Medical and Research Center, Denver, Colo.) was infected with V5-SD4 lentiviruses at a multiplication of infection (MOI) of 10. Two days after infection, GFP-positive cells were enriched 3 times by flow cytometric sorting.

T-cell proliferation assays. To assay effects of heparin on DC-HIL-mediated inhibition, DC-HIL-Fc or control Ig (5 μg/ml) was incubated with heparin at increasing concentrations at room temperature for 30 min, followed by coating ELISA wells (in triplicate) that were precoated with anti-CD3 Ab (0.01-0.3 μg/ml). Purified CD4⁺ T-cells (2×10⁵/well) were added to the coated wells and cultured for 2 d. After pulsing with ³H-thymidine (1 μCi/well) for the last 20-22 h of the culture period, cells were collected and evaluated for ³H-radioactivity. Culture supernatant was stored at −85° C. until needed for IL-2 assay using mouse IL-2 ELISA kit (BD Pharmingen).

Effects of anti-SD-4 Ab on T-cell activation were examined as follows: CD4⁺ T-cells (2×10⁵/well) were treated with biotinylated anti-CD3 Ab at varying concentrations plus biotinylated anti-SD-4 Ab or biotinylated control IgG (10 μg/ml) on ice for 30 min. After adding anti-biotin microbeads (1 μl, Miltenyi Biotec), treated T-cells were incubated in the 96-well-plate and examined for proliferation as described above. For V5-SD4-DO11.10 T-cells, cells (3×10⁴/well) were cultured in 96-wells coated with DC-HIL-Fc (or anti-SD-4 Ab) and anti-CD3 Ab, followed by IL-2 assay.

Expression of SD. Expression of SD was determined by RT-PCR (for mRNA) and flow cytometry (for surface expression). Total RNA was extracted from freshly isolated (or resting) CD4⁺ or CD8⁺ T-cells or from T-cells activated 3 d after treatment with immobilized anti-CD3 Ab, and then subjected to RT-PCR analysis using primers for SDs and β-actin as described previously. Primers for SD-1: 5′-CCCTCCCGCAAATTGTGGCTGTAA-3′ (5′-primer) (SEQ ID NO:10) and 5′-CCCCGTGCGGATGAGATGTGAC-3′ (3′-primer) (SEQ ID NO:11); primers for SD-2: 5′-CCGGGGCGCAGGGAGAA-3′ (SEQ ID NO: 12) and 5′-TTTGGGGGAAGCAGCACTA-3′ (SEQ ID NO:13); primers for SD-3: 5′-CTTGGACACAGAGGCCCCGACACC-3′ (SEQ ID NO:14) and 5′-CGCCCACCACCCCACCCACGAT-3′ (SEQ ID NO:15); and primers for SD-4: 5′-CCTCCCCGACGACGAAGATGC-3′ (SEQ ID NO:16) and 5′-AACGCCCGCCACCCACAAC-3′ (SEQ ID NO:17). The inventors also examined mRNA expression of all members of the glypican family using primers reported previously.

At different time points after activation of CD4⁺ or CD8⁺ T-cells with immobilized anti-CD3 Ab, T-cells were pretreated with Fc blocker and stained with biotinylated anti-SD-4 Ab or biotinylated isotypic control IgG (5 μg/ml) plus PE-streptavidin. Cells were also stained with Ab raised against SD-1 (eBioscience), SD-2, and SD-3 (Santa Cruz Biotechnology, Santa Cruz, Calif.). Finally, PE-labeling intensity was measured by FACS.

Immunoblotting and immunoprecipitation Whole cell extracts were prepared from resting or activated T-cells and subjected to SDS-PAGE/Western blotting using anti-SD-1 or anti-SD-4 Ab (each 5 μg/ml) as described previously. For immunoprecipitation, the protein extracts were incubated with DC-HIL-Fc or control Ig (5 μg/ml) for 3 h at 4EC. Resulting immunocomplexes were precipitated with Protein A-agarose (50 μl of 50% slurry) overnight at 4° C., and washed extensively with PBS. The complexes were dissociated by boiling and then analyzed for SD-4 expression by Western blotting using rat anti-SD-4 Ab (5 μg/ml) and HRP-anti-rat IgG (1:10,000 dilution).

### Example 5

### Results

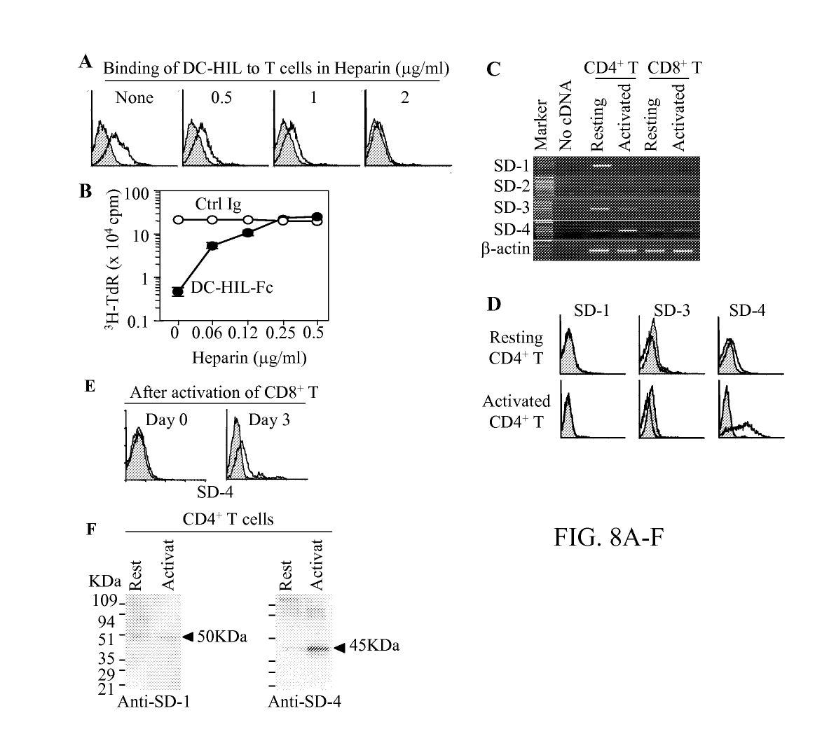

Heparin antagonizes DC-HIL function. Having shown that DC-HIL binds to heparin/heparan sulfated polysaccharides, the inventors considered a role for these polysaccharides in the interaction between DC-HIL and activated T-cells. Pretreatment of DC-HIL-Fc with heparin blocked binding of this soluble receptor to activated T-cells in a dose-dependent fashion (FIG. 8A). To assess effects on the inhibitory function of immobilized DC-HIL, T-cells were cultured in microwells precoated with anti-CD3 Ab and with control Ig or DC-HIL-Fc pretreated with heparin. Pretreatment with heparin abrogated the ability of DC-HIL to inhibit T-cell activation triggered by anti-CD3 Ab (FIG. 8B). Heparin alone had no effect on anti-CD3 Ab response. Similar results were noted using heparan sulfate (data not shown). The ability of exogenous heparin to block binding of DC-HIL to T-cells and to antagonize its inhibitory function indicates a role for heparin/heparan sulfate in these processes.

Syndecan-4 on activated T-cells is a ligand of DC-HIL. Because the syndecan (SD) and glypican families of transmembrane heparin/heparan sulfate proteoglycans (HSPG) are major sources of cell surface-heparan sulfate, the inventors questioned whether these molecules are involved in binding of DC-HIL to T-cells. The inventors first examined expression by resting versus activated CD4⁺ or CD8⁺ T-cells of all known SDs and glypicans. At the mRNA level, all 4 known SDs were expressed by resting and activated T-cells at differing levels, but only SD-4 expression was upregulated by T-cell activation (FIG. 8C). None of the 6 known glypicans was expressed by T-cells (data not shown). At the surface protein level, SD-1 and SD-3 were not expressed but their mRNAs were detected by RT-PCR (FIG. 8C). Again, SD-4 was the sole HSPG whose expression was upregulated by T-cell activation (FIG. 8D). SD-4 expression on CD8⁺ T-cells was also induced by activation, albeit to a lesser degree than observed with CD4⁺ T-cells (FIG. 8E). Activated T-cells produced SD-4 proteins at greater levels than resting T-cells (FIG. 8F); de novo protein synthesis (rather than just movement of the protein to the cell surface) is likely to account for this activation-inducible expression of SD-4. The expression profile of SD-4 was consistent with that of DC-HIL binding.

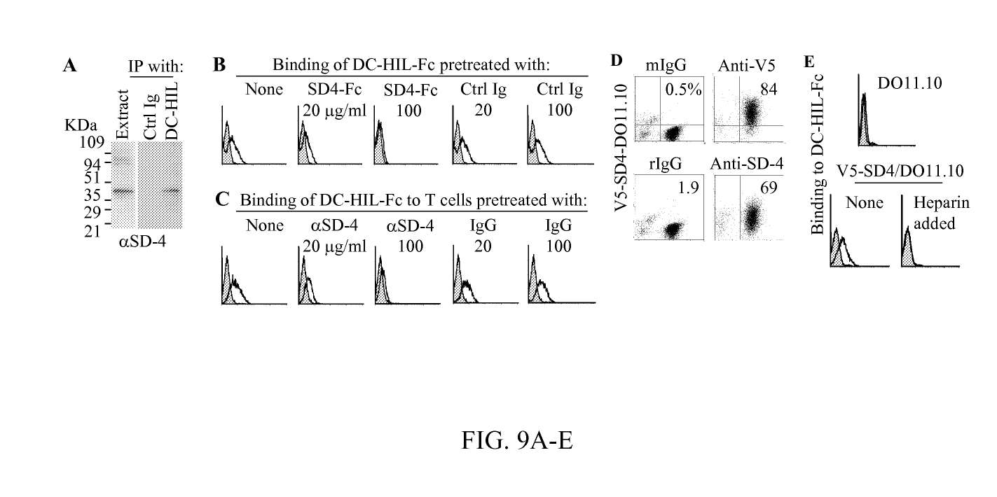

To determine whether DC-HIL binds directly to SD-4 on activated T-cells, the inventors extracted proteins from activated CD4⁺ T-cells, immunoprecipitated these with DC-HIL-Fc or control Ig, and analyzed precipitants by Western blotting using anti-SD-4 Ab (FIG. 9A). A single band of SD-4 (45 kDa) was detected in DC-HIL-bound (but not control Ig-bound) immunoprecipitates. The inventors then questioned whether SD-4 is the sole ligand of DC-HIL on activated T-cells. Pretreatment of DC-HIL-Fc with SD4-Fc (soluble receptor fused with Fc portion of IgG) or pretreatment of activated T-cells with anti-SD-4 Ab abrogated binding of DC-HIL to T-cells (FIGS. 9B and 9C); control Ig had no effect. Finally, transgene expression of SD-4 in DO11.10 T-cells (lacking native SD-4 expression) conferred on these cells the ability to bind DC-HIL (FIGS. 9D and 9E), and this binding was also blocked completely by heparin. These results indicate that SD-4 is the major (if not sole) ligand of DC-HIL on activated T-cells.

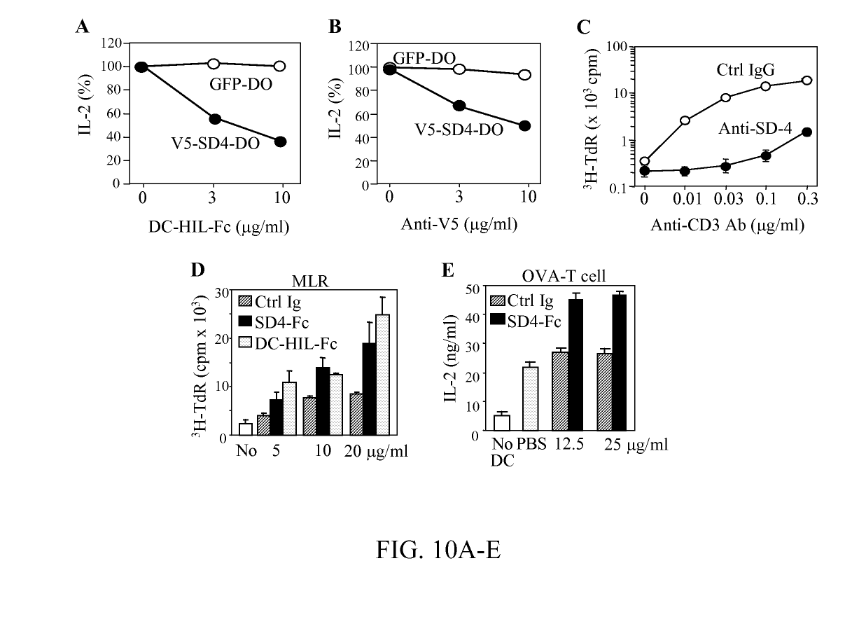

Engagement of SD-4 leads to inhibition of T-cell activation. The inventors examined the function of SD-4 on T-cells again using transfected DO11.10 T-cells. V5-SD4-expressing DO11.10 T-cells (V5-SD4-DO) or those expressing GFP (GFP-DO) were treated with immobilized anti-CD3 Ab in the presence/absence of DC-HIL-Fc, followed by measurement of IL-2 production (FIG. 10A). DC-HIL-Fc inhibited anti-CD3-induced IL-2 production by V5-SD4-DO cells in a dose-dependent fashion, but did not inhibit IL-2 production by GFP-DO cells. In the absence of DC-HIL, both cell lines showed similar anti-CD3 responses (FIG. 10A), indicating that expression of V5-SD4 had no effect on the T-cell response. Cross-linking of SD-4 on T-cells also led to inhibited IL-2 production, albeit to a lesser degree (FIG. 10B).

The inventors also examined the inhibitory function of SD-4 on CD4⁺ T-cells (FIG. 10C). CD4⁺ T-cells were activated by cross-linking of CD3 (using increasing doses of anti-CD3 Ab) and SD-4 (constant dose of anti-SD-4 Ab). T-cells treated with anti-SD-4 Ab (but not isotypic IgG control) exhibited very low responses to anti-CD3-stimulation at each dose tested (FIG. 10C). Cross-linking of SD-4 produced outcomes mimicking the inhibitory function of DC-HIL.

Soluble SD-4 enhances T-cell activation. The inventors next compared effects of SD4-Fc and DC-HIL-Fc on activation of alloreactive T-cells (mixed lymphocyte reaction or MLR), in which added Fc-fusion proteins were used to block endogenous binding of DC-HIL on APC with SD-4 on T-cells (FIG. 10D). Addition of SD4-Fc to the MLR led to enhanced T-cell activation (2-fold higher than control) in a dose-dependent fashion, as shown previously using DC-HIL-Fc. The inventors also added soluble SD4-Fc to a syngeneic APC assay, in which OVA peptide-pulsed bone marrow-derived DC (BM-DC) were co-cultured with splenic CD4⁺ T-cells isolated from DO11.10 transgenic mice (FIG. 10E). SD4-Fc enhanced T-cell activation triggered by DC, whereas control Ig had little effect. Since SD4-Fc acted as an antagonist (i.e., it can bind to DC-HIL on DC, but unable to induce tyrosine phosphorylation of DC-HIL in DC) (data not shown), the inventors interpret these results to mean that SD-4 is a negative regulator of allogeneic and syngeneic T-cell responses.

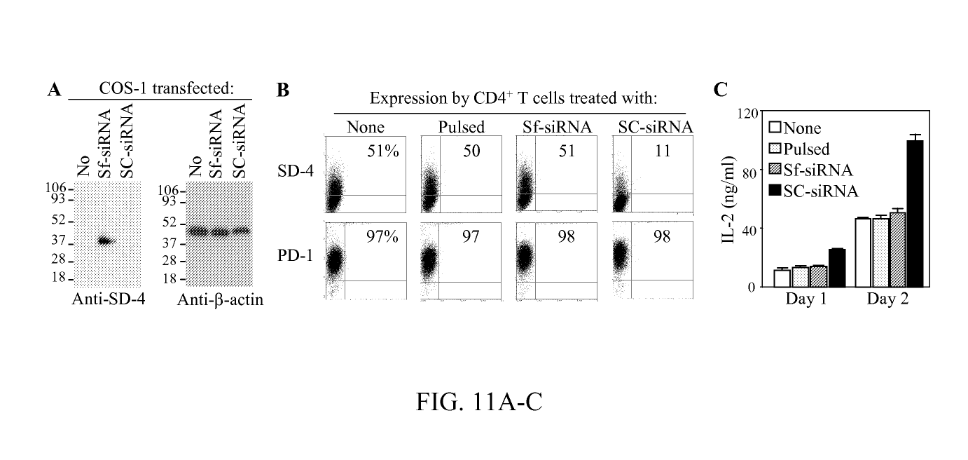

T-cells with knocked-down SD-4 display enhanced responses to DC. To better study the role of SD-4 in regulating T-cell activation, the inventors knocked-down SD-4 expression on splenic CD4⁺ T-cells and examined their response to antigen presentation by DC. The inventors first determined the efficacy of siRNA's ability to block SD-4 expression in COS-1 cells co-transfected with SD-4 gene and SD-4-targeting SC-siRNA or shuffled control siRNA (Sf-siRNA), using Western blotting for protein expression of SD-4 transgene or endogenous β-actin (as control) (FIG. 11A). SC-siRNA knocked-down SD-4 protein expression almost completely (control siRNA had no effect); specificity was supported by unchanged β-actin expression. The inventors then transfected both siRNA into CD4⁺ T-cells freshly isolated from DO11.10 transgenic mice (FIG. 11B), which resulted in 60% delivery of siRNA into cells (data not shown). Transfection of SC-siRNA knocked-down SD-4 expression induced by activation (51% reduced to 11%); control siRNA had no effect. Specificity of SC-siRNA was supported by no effect on PD-1 expression by activated T-cells as a control. T-cells were then evaluated for response to activation by DC pulsed with OVA peptide. A day after co-culture with Ag-pulsed DC, T-cells transfected with SC-siRNA produced 2-fold higher levels of IL-2 compared to that produced by control T-cells (untreated versus pulsed alone versus transfected with control siRNA) (FIG. 11C). Similar results were observed after 2 days of co-culture. These results indicate that down-regulated SD-4 expression enhances T-cell responses to activation signals delivered by DC.

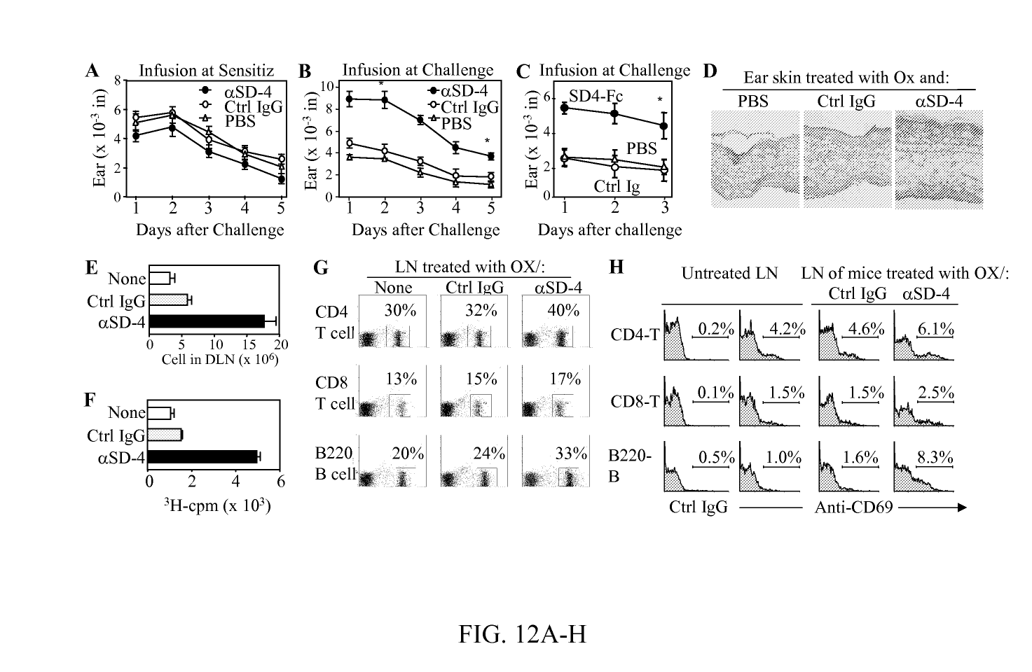

Blockade of endogenous SD-4 augments contact hypersensitivity (CH). To evaluate SD-4 function in vivo, the inventors employed CH, an experimental model of delayed-type, T-cell-mediated, skin inflammation. Mice were sensitized by topical application of oxazolone (Ox) on abdominal skin and challenged by painting ear skin with Ox (FIGS. 12A-H); CH was measured by ear swelling from days 0 through 5. Mice were given an intraperitoneal injection of anti-SD-4 Ab (versus PBS vs. control IgG) 3 h prior to sensitization (FIG. 12A) or challenge (FIG. 12B). Infusion of anti-SD-4 Ab prior to sensitization had little effect on ear swelling (FIG. 12A). By contrast, mice treated with anti-SD-4 Ab prior to challenge developed twice greater ear swelling compared to controls at each time point tested after challenge (FIG. 12B); this enhancement lasted at least 5 days after challenge. Similar enhancement was seen following infusion of SD4-Fc at challenge (FIG. 12C). These time-dependent effects were consistent with previous outcomes using DC-HIL-Fc. The inventors interpret these results to mean that SD-4 is expressed optimally on fully activated T-cells (after challenge) but not yet on early activated T-cells (during sensitization).

Histological examination confirmed enhanced ear swelling and larger numbers of infiltrating leukocytes in mice treated with Ox and anti-SD-4 Ab (FIG. 12D). The inventors next examined the phenotype of draining lymph nodes (LN) in mice similarly treated with Ox and anti-SD-4 Ab 2 days after challenge including: cell numbers (FIG. 12E); spontaneous activation measured by ³H-thymidine incorporation for proliferative capacity in vitro (FIG. 12F); and frequency of leukocyte subpopulations (FIG. 12G) and of CD69⁺ cells measured by FACS (FIG. 12H). In mice treated with anti-SD-4 Ab (vs. control IgG), LN size was almost 3× bigger and spontaneously proliferating LN cells 3-fold greater; there were more CD4⁺ T-cells (40% vs. 32%), CD8⁺ T-cells (17% vs. 15%), and B220+B cells (33% vs. 24%); and the frequency of CD69⁺ cells in all three leukocytes increased. In sum, the ability of anti-SD-4 Ab (or SD4-Fc) to recapitulate DC-HIL function (inhibition of T-cell activation in vitro while augmenting MLR, antigen presentation, and CH responses) strongly support SD-4 as the ligand through which DC-HIL mediates its inhibitory signal in T-cells.

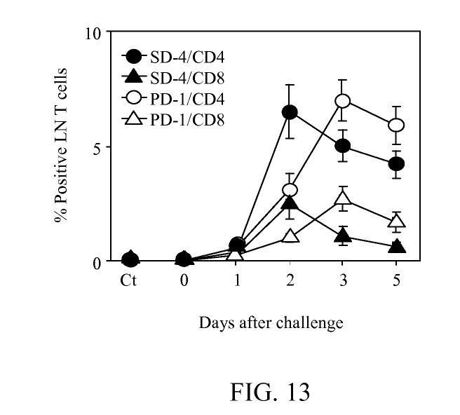

Expression of SD-4 on LN T-cells. The time-dependent effect of anti-SD-4 Ab in mice (FIGS. 12A-H) led the inventors to examine the kinetics of SD-4 expression by T-cells during development of CH (FIG. 13). The inventors also compared SD-4⁺ vs. PD-1⁺ T-cells in LN from control mice vs. those treated with Ox at different time points after hapten challenge. In Ox-sensitized mice, SD-4⁺ T-cells were not detected in LN prior to challenge. A day after challenge, both CD4⁺ and CD8⁺ T-cells expressed SD-4 at low frequency, that increased dramatically 2 days after challenge (6.6% in CD4⁺ cells and 2.5% in CD8⁺ cells). This was followed by a gradual decrease to 4.2% in CD4⁺ and 0.6% in CD8⁺ cells 5 days after challenge. The frequency of PD-1⁺ T-cells also increased after hapten challenge, peaking at day 3. The expression of SD-4 in LN T-cells after challenge (but not after sensitization) likely accounts for the ability of anti-SD-4 Ab or soluble SD4-Fc to augment CH responses when injected during elicitation (but not sensitization) (FIGS. 12A-C).

### Example 6

### Discussion

These results demonstrate that SD-4 is the ligand through which DC-HIL inhibits T-cell activation. Among known co-inhibitory receptors, SD-4 resembles PD-1 in that expression requires TCR activation and appears during a later phase of T-cell activation (e.g., elicitation of CH). Unlike all known co-inhibitory receptors including PD-1 that bind their counter-receptors via protein-protein interaction, binding of SD-4 to DC-HIL appears to require involvement of heparin/heparan sulfate residues.

Because binding of DC-HIL to activated T-cells involves its Ig-like PKD domain and because DC-HIL does not bind to CD44, another HSPG expressed on resting and activated T-cells (data not shown), the inventors hypothesize that DC-HIL/SD-4 binding requires simultaneous recognition of heparin/heparan sulfate and of a peptide epitope of SD-4. This circumstance may resemble selectins, which bind the carbohydrate moiety, sialyl-Lewis×(SLex), and a peptide sequence on the backbone of its ligands.

Because endothelial cells and B cells consitutively express SD-4, it is possible such cells are also stimulated by DC-HIL. The inventors have shown DC-HIL to bind endothelial cells in an heparin-dependent fashion. By contrast, B cells do not bind DC-HIL (data not shown); reflecting a discordance between expression and binding activity that may be due to diverse heparan sulfate structures expressed by disparate cells, in turn corresponding to different binding activities. DC-HIL may recognize a unique heparan sulfate structure on SD-4 synthesized by activated T-cells, similar to T-cell-specific glycosylation again exhibited by the selectin ligands.

The enhancing effect of anti-SD-4 Ab or soluble recombinant SD4-Fc on CH indicates interference with endogenous binding of DC-HIL to SD-4. A good question is whether such enhancement is due entirely to blockade of DC-HIL/SD-4 binding since SD-4 participates in leukocyte rolling and migration. If SD-4 antagonists block the latter processes, these experiments should have produced down-regulated CH instead. That these outcomes were the reverse indicate that the primary target of the inventors' interventions is endogenous binding of DC-HIL to SD-4, and thus modulators of DC-HIL and/or SD-4 may be used to treat T-cell-mediated diseases.

BTLA, CTLA-4, and PD-1 possess a typical ITIM, an ITIM-like motif, and an immunoreceptor tyrosine-based switch motif (ITSM), respectively, that can activate the tyrosine phosphatases, SHP-1 and SHP-2, responsible for mediating negative T-cell effector function. By contrast, SD-4 does not contain any of these inhibitory motifs. Rather, ligated SD-4 is known to induce serine and tyrosine autophosphorylation, which may regulate intracellular interactions of SD-4 with other cell surface receptors. Although the inventors have no direct evidence connecting these events to the SHP-1/SHP-2 pathway in T-cells, activated SD-4 has been shown to complex with other intracellular proteins like syntenin that can bind directly to the intracellular domain of CD148, a membrane protein tyrosine phosphatase (PTPη) known to inhibit CD3-mediated T-cell activation. Moreover, SD-4 can regulate activation of PKCα, that in turn can modulate phosphorylation of CD148. Finally, the inventors speculate that ligated SD-4 on T-cells partners with CD148 to activate tyrosine phosphatases, which can lead to neutralization of TCR-induced activation signals.

### Example 7

### Materials and Methods

Plasmids and preparation of DC-HIL-Fc. A plasmid vector encoding SD-1 or SD-4 was constructed by inserting the full-length cDNA into pcDNA3.1 vector (Invitrogen, Carlsbad, Calif.) using HindIII and XbaI restriction enzyme sites. The inventors also constructed pcDNA-SD-4-V5 by attaching the V5 epitope sequence (Sato et al., 2006) to the 3′-end of the SD-4 cDNA insert. A plasmid vector encoding the extracellular domain of human DC-HIL fused to the Fc portion of mouse IgG₂ₐ (DC-HIL-Fc) was constructed by replacing the extracellular domain of mouse homolog in pSTB-DC-HIL-Fc (Chung et al., 2007a) with the corresponding of human homolog. Fc proteins were produced in COS-1 cells and purified using protein-A-agarose (Shikano et al., 2001).

Ab and immunofluorescence labeling. Mouse anti-human DC-HIL mAb was generated by immunizing BALB/c mice with human DC-HIL-Fc at 2 week-intervals. A week after the last immunization, spleen cells from mice with highest Ab titer were fused with the F/0 myeloma cell line. One 3D5 IgG₁ clone was purified from mouse ascites using protein-A affinity chromatography.

mAb against CD1a (HI149), CD3 (UCHT1), CD14 (61D3), CD28 (CD28.2), CD69 (FN50), CD80 (2D10.4), CD86 (FUN-1), HLA-DR (LN3), PD-1 (MIH4) and SD-1 (DL-101) were purchased from eBiosciences (San Diego, Calif.); Ab against SD-2 (M-140), SD-3 (M-300), SD-4 (H-140 and 5G9) and p-SD-4 (Ser 179) from Santa Cruz Biotechnology (Santa Cruz, Calif.); anti-V5 Ab from SeroTec (Raleigh, N.C.); and secondary Ab from Jackson ImmunoResearch (West Grove, Pa.). For flow cytometric analysis, cells (5−10×10⁵) were incubated with 5-10 μg/ml primary Ab for 30 min on ice, followed by addition of secondary Ab (2.5 μg/ml). After washing, cell-bound fluorescence was analyzed by FACSCalibur (BD Biosciences, San Jose, Calif.).

Binding of DC-HIL to T cells. After providing informed consent, blood was collected from healthy donors and PBMC isolated by Ficoll-Hypaque gradient centrifugation. Following manufacture's recommendations, CD4+ or CD8+ T cells (1×10⁶) were isolated from PBMC using respective isolation kits (Myltenyi Biotec, Auburn, Calif.) and cultured with concanavalin A (Con A, 2 μg/ml), phytohemagglutinin (PHA, 5 μg/ml), phorbol 12-myristate 13-acetate (PMA, 5 ng/ml) plus ionomycin (250 ng/ml) (all from Sigma, St. Louis, Mo.), or anti-CD3 Ab (2 μg/ml) plus anti-CD28 Ab (0.5 μg/ml). At indicated time points after culturing, activated T cells (1×10⁶) were pretreated with 5 μg/ml human IgG on ice for 30 min to block Fc-binding activity on T cells prior to incubation with 10 μg/ml DC-HIL-Fc or control Ig plus 2.5 μg/ml PE-anti-mouse IgG F(ab′)2.

T cell activation. Freshly-isolated CD4+ or CD8+ T cells (2×10⁵/well) were cultured for 2 d in ELISA wells (in triplicate) precoated with indicated doses of anti-CD3 Ab and together with Fc proteins or Ab. CD4+ T cells were also incubated in ELISA wells precoated with DC-HIL-Fc (10 μg/ml)/anti-CD3 Ab (0.3 μg/ml) and increasing doses of anti-CD28 Ab. T cell activation was measured by 3H-thymidine incorporation (pulsing with 1 μCi/well in the last 20 h of the culture period) or by IL-2, TNF-α, and IFN-γ production using ELISA kit (eBioscience).

For the mixed lymphocyte reaction (MLR), PBMC were γ-irradiated (2,000 Gy) and mixed with CD4+ T cells (2×10⁵/well) in 96-microwell-plate (in triplicate) at indicated ratios (PBMC vs. T cells) in the absence/presence of DC-HIL-Fc or control Ig (5 μg/ml) for 4 d (Chung et al., 2007a).

For allostimulatory assays, CD14+ monocytes were isolated from PBMC of a healthy donor using anti-CD14 Ab magnetic beads (Miltenyi Biotec) and cultured with/without TGF-β (10 ng/ml) for 2 d. Cells were then harvested and cocultured in 96-well plates with CD4+ T cells (from a different donor) at the indicated cell ratio for 6 d [35]. In some experiments, the cell mixture was cultured in the presence of DC-HIL-Fc or control Ig (20 μg/ml).

Cell cycle analysis. Cell cycles of CD4+ T cells treated with anti-CD3 Ab (0.3 μg/ml) and Fc protein (5 μg/ml) were analyzed using FITC-BrdU flow kit (BD Pharmingen) (Chung et al., 2007a).

RT-PCR. Total RNA was extracted from leukocytes, reverse-transcribed to cDNA, and PCR-amplified using primers for DC-HIL (5′-primer, 5′-GTGGAGCTTCGGGGATAATACT-3′ (SEQ ID NO:18); and 3′-primer, 5′-CTACTCAGCTCCAGGGGGTTGT-3′ (SEQ ID NO:19)), SD members, and GAPDH (Wegrowski et al., 2006).

Stable transfectants. Jurkat T cell E6-1 line (1×10⁶ cells) was transfected with an empty vector, pcDNA-SD-1 or pcDNA-SD-4 (2 μg), using the Amaxa Nucleofector System (Gaithersburg, Md.). 2 d post-transfection, cells were allowed to grow in the selection media containing 600 μg/ml G418 (Invitrogen) for 2 weeks. Jurkat cells expressing SD-1 or SD-4 were then enriched by FACS-sorting until more than 90% of cells are positive for surface expression. Control Jurkat cells (transfected with vector alone) were established by culturing with G418 more than 3 weeks. For binding of DC-HIL, Jurkat cells were activated with Con A (2 μg/ml) for 2 d prior to the binding assay. For experiments examining involvement of heparin, binding of DC-HIL-Fc (10 μg/ml) to Con A-activated Jurkat cells was performed in the presence of heparin, or Jurkat cells (5×10⁵) were treated with heparinase I (0.1 U/ml) and III (0.2 U/ml) (Sigma) at 37° C. for 2 h before binding assay. To examine specificity of SD-4 binding to DC-HIL, activated Jurkat cells (5×10⁵) were pretreated with anti-SD-4 or control IgG at indicated concentrations for 30 min at room temperature before binding. For activation assay, Jurkat T cells (3×10⁴/well) were cultured for 2 d in ELISA wells (in triplicate) precoated with indicated doses of anti-CD3 Ab and DC-HIL-Fc or control Ig (each 10 μg/ml).

Immunoprecipitation and tyrosine phosphorylation assay. Whole cell extracts (1×10⁷ cells/ml) were prepared from activated CD4+ T cells (Sato et al., 2006) and incubated with DC-HIL-Fc or control Ig (5 μg) for 3 h at 4° C. Resulting immunocomplexes were precipitated with protein A-agarose (50 μl of 50% slurry) overnight at 4° C., and washed extensively with PBS. To assay phosphorylation of SD-4, Jurkat cells were transfected with pcDNA-SD4-V5 and immediately cultured with Con A (2 μg/ml) for 2 d. After culturing for another 1 d without Con A, cells (2×10⁶) were cultured in a petri dish precoated with DC-HIL-Fc (20 μg/ml) at 37° C. for different time period. Whole cell extracts were prepared, incubated with anti-V5 Ab (2 μg/ml) at 4° C. for 3 h, and then precipitated with 50 μl of 50% slurry protein G-agarose (Pierce, Rockford, Ill.) by overnight incubation. After washing, agarose-beads were then left untreated (to detect phosphoserine) or treated (to detect phosphotyrosine) with a mixture of heparinase I (0.1 U/ml) and III (0.2 U/ml), and chondoroitinase ABC (Sigma) (Charnaux et al., 2005) prior to immunoblotting using anti-phosphorylated Ser-179 of SD-4 or biotinylated anti-phospho-tyrosine (0.5 μg/ml) (4G10, Upstate, Lake Placid, N.Y.) and HRP-streptavidin (1:10,000). Blotted membranes were also stripped and re-probed with mouse anti-SD-4 Ab (1 μg/ml) and HRP-anti-mouse IgG (1:10,000).

Culture of monocytes and DC. PBMC were cultured in 24-well plates (1×10⁶ cells/well in triplicate) for 2 d with 10% FCS-RPMI supplemented with IL-2 (100 U/ml), IL-4, IL-6, IL-10, IL-11 (each at 10 ng/ml), IL-13 (100 U/ml), IFN-γ (200 U/ml), TNF-α, TGF-β (each at 10 ng/ml) (all from PeproTech Inc, Rocky Hill, N.J.), or LPS (1 μg/ml) (Sigma). For generation of monocyte-derived immature DC [37], PBMC were seeded onto tissue culture flasks. After culturing for 1 h, non-adherent cells were washed off and remaining adherent cells cultured in DC culture media (10% FCS-RPMI supplemented with 800 U/ml GM-CSF and 250 U/ml IL-4) for 6 d. Resulting non-adherent cells were used as immature DC. For induction of maturation, DC harvested from the day 4 culture of adherent PBMC were cultured in 24-well plates (1×10⁶/well) for another 2 d with DC culture media added with IL-1β (10 ng/ml), TNF-α (10 ng/ml), and prostaglandin E2 (PGE2, 1 μg/ml). For LC, epidermal cells were prepared from foreskin and LC identified as CD 1a+ epidermal cells (Takao et al., 2002).

Knock-down of DC-HIL expression. DC-HIL-targeted siRNA (Cat#sc-60721) and control siRNA (Cat#sc-37007, Santa Cruz Biotechnology) (each 2 μg) was treated with 15 μl of Metafetene™Pro (Biotex, Martinsried, Germany) in the serum-free RPMI for 30 min and then added to 1×10⁶ CD 14+ cells. After culture for 4 h at 37° C., cells were washed and cultured in 10% FCS-RPMI for another 2 d before experiments.

Statistical analysis. Results are presented as means±s.d. of n independent experiments. Significance was assessed using the Student's t test at p<0.05.

### Example 8

### Results

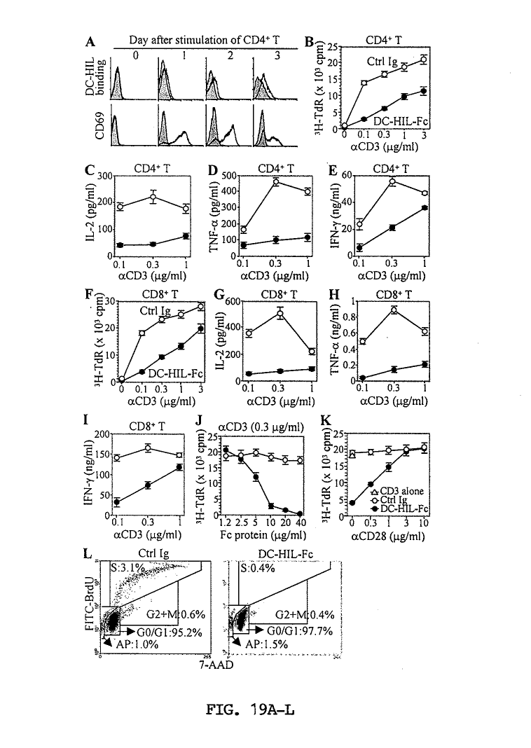

Binding of DC-HIL to human T cells attenuates responses to anti-CD3 Ab. To examine the function of human DC-HIL, the inventors created a soluble DC-HIL receptor (DC-HIL-Fc) consisting of the extracellular domain fused to the Fc portion of mouse IgG, and examined binding to T cells (FIG. 19A). At different time points following PMA/ionomycin stimulation, CD4⁺ T cells were incubated with DC-HIL-Fc or control Ig; cell-bound DC-HIL-Fc was measured by flow cytometry. Activation status was assayed via surface expression of CD69 (an early activation marker). DC-HIL-Fc did not bind resting (freshly isolated) T cells. Rather, it bound to T cells starting 2 days after stimulation, peaking on day 3 when CD69 expression started to decline. Other stimuli (Con A and anti-CD3/anti-CD28 Ab) similarly induced DC-HIL binding (data not shown). These results suggest that the DC-HIL ligand on T cells is expressed at a later stage of T cell activation. Similar results were observed with CD4+ T cells from 3 different donors and with CD8+ T cells (data not shown). To characterize effects of DC-HIL-binding on T cell activation, the inventors cultured CD4+ or CD8+ T cells in 96-microwells co-immobilized with anti-CD3 Ab (increasing doses) and DC-HIL-Fc or control Ig (constant doses), followed by 3H-thymidine incorporation to measure proliferation (FIG. 19B). Immobilized anti-CD3 Ab stimulated CD4+ T cells to proliferate strongly in a dose-dependent manner. Such proliferation was attenuated markedly (10-fold decrease at a dose of 0.1 μg/ml anti-CD3 Ab) by co-treatment with DC-HIL-Fc (but not with control Ig).

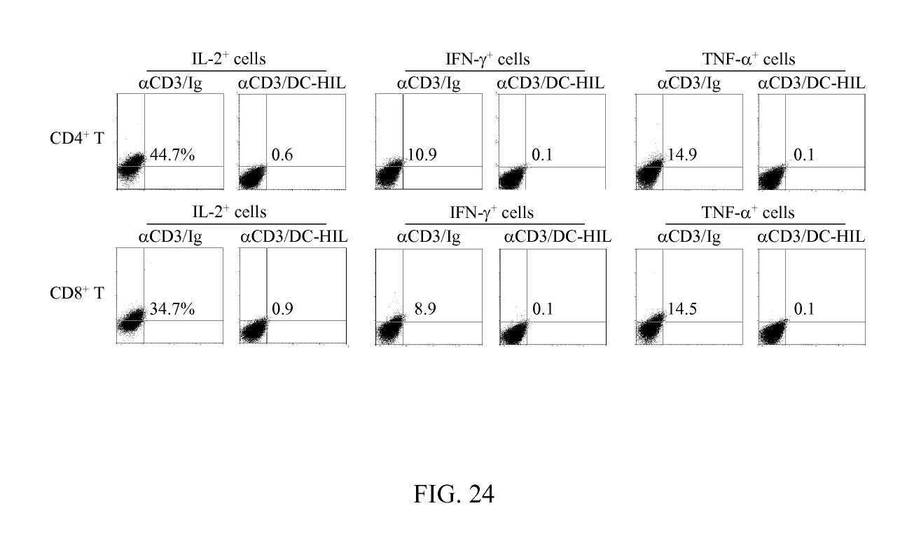

The inventors next examined effects of DC-HIL on cytokine production. Co-treatment of T cells with DC-HIL markedly inhibited production of IL-2 and TNF-α induced by anti-CD3 Ab (FIGS. 19C and D); IFN-7 production was also inhibited but to a lesser degree (FIG. 19E). DC-HIL also strongly inhibited proliferation of CD8+ T cells triggered by anti-CD3 Ab (FIG. 19F). Production of all 3 cytokines tested was markedly inhibited (FIG. 19G-I). Intracellular cytokine staining revealed inhibited cytokine production for all T cells with no significant change in cell number, indicating a functional effect by DC-HIL (FIG. 24). Similar results were noted for CD4+ and CD8+ T cells from 3 other healthy donors (data not shown). To examine dose-response of DC-HIL to a suboptimal dose of anti-CD3 Ab, increasing doses of DC-HIL was added to a constant dose of anti-CD3 Ab (0.3 μg/ml) (FIG. 19J). Strong inhibition required at least 5 μg/ml of DC-HIL-Fc and this inhibition was rescued by co-treatment with anti-CD28 Ab in a dose-dependent manner (FIG. 19K). Finally, the inventors examined effects of DC-HIL treatment on the cell cycle of activated T cells (FIG. 19L). The inventors cultured CD4+ T cells with immobilized anti-CD3 Ab plus DC-HIL-Fc or control Ig for 2 days, and assayed the T cells for DNA content (staining with 7-AAD) and proliferation (incorporation of BrdU stained with FITC-anti-BrdU Ab) by flow cytometry on a per cell basis. Based on ratios of fluorescence intensities of 7-AAD and FITC-BrdU, respectively, the distribution of T cells treated with anti-CD3 Ab/control Ig was: 95.2% in G0/G1 phase; 0.6% G2/M phase; 3.1% S phase; and 1.0% subdiploid (including cells headed for apoptosis). T cells treated with anti-CD3 Ab/DC-HIL-Fc sorted to similar portions except for markedly less cells in the S phase (0.4%). Altogether, binding of DC-HIL to T cells led to attenuated T cell responses to anti-CD3 Ab, accompanied by reduced cytokine secretion (IL-2, TNF-α, and IFN-γ) and blocked entry into the S phase.

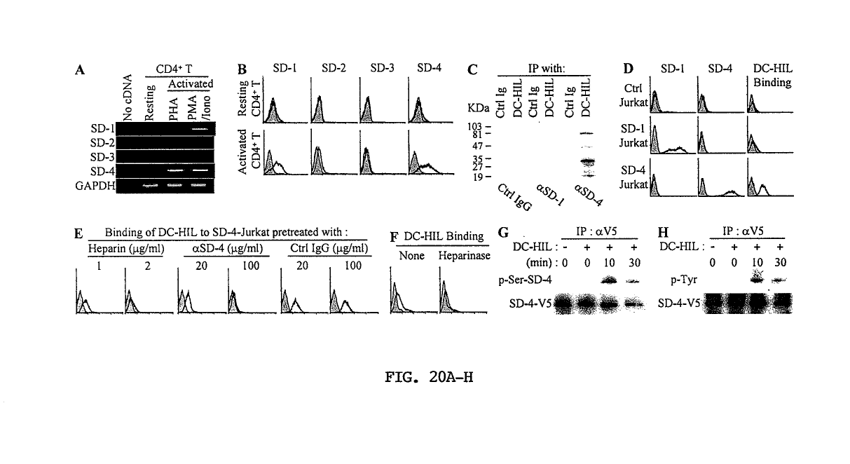

SD-4 is the T cell ligand of DC-HIL Having identified SD-4 as the ligand of DC-HIL in mice (Chung et al., 2007a), the inventors wished to ascertain a similar circumstance in humans. The inventors first examined mRNA and protein expression of the 4 known syndecans (SDs) by RT-PCR and flow cytometry, respectively. By RT-PCR analysis (FIG. 20A) and as predicted by studies in mice, SD-4 was expressed by activated (but not resting) T cells. Unlike mouse T cells, however, human CD4+ T cells also expressed SD-1 mRNA following activation with PMA/ionomycin (but not with PHA). Surface expression of both SD-1 and SD-4 on T cells was induced by activation with PMA/ionomycin (FIG. 20B). Similar findings were shown for CD8+ T cells (data not shown). By contrast, neither SD-2 nor SD-3 was expressed by T cells, even after activation.

The inventors next examined binding of DC-HIL to SD-4 and SD-1. Protein extracts from activated T cells were treated with DC-HIL-Fc or control Ig, and immunoprecipitates blotted with anti-SD-1, anti-SD-4 Ab, or control IgG (FIG. 20C). DC-HIL-Fc-precipitates contained SD-4 but not SD-1. The presence of multiple bands of SD-4 is due likely to different levels of glycosylation (Chamaux et al., 2005; Woods and Couchman, 2001). Specificity was indicated by failure of control Ig to precipitate SD-4.

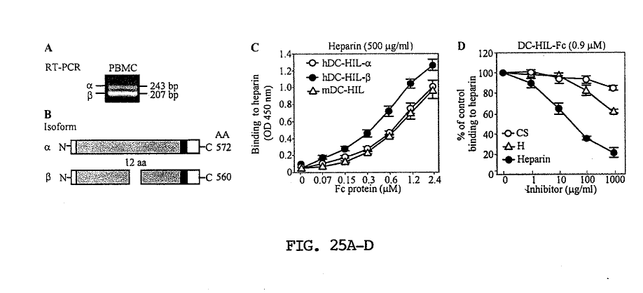

Taking advantage of the Jurkat T cell line naturally devoid of endogenous SD-1 and SD-4, the inventors transfected the SD-1 or SD-4 gene into these cells, and confirmed high cell surface expression of either gene in the respectively engineered cells (FIG. 20D). Surprisingly, neither SD-4+ nor SD-1+ Jurkat cells bound to DC-HIL. However, treatment with Con A induced binding of DC-HIL to SD-4+ (but not SD-1+) Jurkat cells. Specificity of binding to SD-4 was confirmed by pretreatment of Jurkat cells with anti-SD-4 Ab and by inability of control IgG to inhibit the binding (FIG. 20E). The requirement of a high concentration for blocking is due likely to the high expression of SD-4 on these Jurkat cells. Because DC-HIL binds to heparin (FIGS. 25A-D), the inventors examined a role for heparin in this process. Binding of DC-HIL was abrogated completely with addition of heparin to the binding assay (FIG. 20E). Moreover, pretreatment of SD-4+ Jurkat cells with heparinase abrogated binding of DC-HIL to the cells (FIG. 20F). These results indicate that human SD-4 (but not SD-1) is a binding partner of DC-HIL and its saccharide moiety (sensitive to heparinase) participates in the binding.

Binding of DC-HIL to SD-4 induces serine and tyrosine autophosphorylation. Because SD-4 autophosphorylates its intracellular tyrosine and serine residues following binding to ligands (Horowitz and Simons, 1998), the inventors ascertained a similar scenario for binding to DC-HIL. Jurkat cells were transfected transiently with a gene for V5-tagged SD-4 (SD4-V5) and stimulated with Con A before incubation with immobilized DC-HIL-Fc. At different time points after incubation, Jurkat cells were assayed for phosphorylation using immunoprecipitation of SD4-V5 with anti-V5 Ab, followed by immunoblotting with Ab to phosphorylated serine of SD-4 (FIG. 20G) or phosphotyrosine (FIG. 20H). Within 10 min after treatment, serine and tyrosine residues were phosphorylated, indicating that ligation to DC-HIL triggered SD-4-dependent signals.

Engagement of SD-4 attenuates anti-CD3 response of T cells. To study the function of SD-4 on T cells, Jurkat transfectants were incubated with co-immobilized anti-CD3 Ab (different doses) and DC-HIL-Fc or control Ig (a constant dose). Activation was measured by IL-2 production (FIG. 21A). Without DC-HIL, all 3 Jurkat transfectants (control, SD-1+ and SD-4+ cells) produced similar levels of IL-2 at a highest dose of anti-CD3 Ab. Co-immobilization of DC-HIL had very little effect on IL-2 production by SD-1+ Jurkat cells (and by control cells), whereas it strongly reduced IL-2 production by SD-4+ Jurkat cells. These results are consistent with the inability of SD-1+ Jurkat cells to bind DC-HIL and document SD-4 to mediate the inhibitory function of DC-HIL.

To evaluate the effect of anti-SD-4 Ab on the anti-CD3 response of CD4+ T cells, cells were cultured in microculture wells precoated with anti-SD-4, anti-SD-1, DC-HIL-Fc, or control Ig (FIG. 21B). T cell activation was measured by proliferative capacity. Again, immobilized DC-HIL-Fc markedly blocked proliferation of T cells activated by anti-CD3 Ab. To an even higher level, immobilized anti-SD-4 Ab blocked activation, whereas anti-SD-1 Ab did not. The inventors then compared this inhibitory effect to those of other inhibitory receptors using the same assay (FIG. 21C). Reproducibly, anti-SD-4 Ab almost completely abrogated proliferation of T cells triggered by 0.1 μg/ml of anti-CD3 Ab, with this inhibition still manifested at the highest dose (3 μg/ml) tested. By contrast, treatment with anti-PD-1 or anti-CTLA-4 Ab reduced proliferation by 50% at a dose of 0.1 μg/ml anti-CD3 Ab and 10% at the highest dose. Proliferation induced by a suboptimal dose of anti-CD3 Ab (0.3 μg/ml) was titrated with increasing doses of the 3 Ab (FIG. 21D). Anti-CTLA-4 and anti-PD-1 Ab at the highest dose (40 μg/ml) displayed 10% and 50% inhibition, whereas anti-SD-4 Ab inhibited the proliferation dose-dependently (completely at 40 μg/ml). Altogether, these results buttress the concept of SD-4 as a highly potent inhibitor of T cell activation.

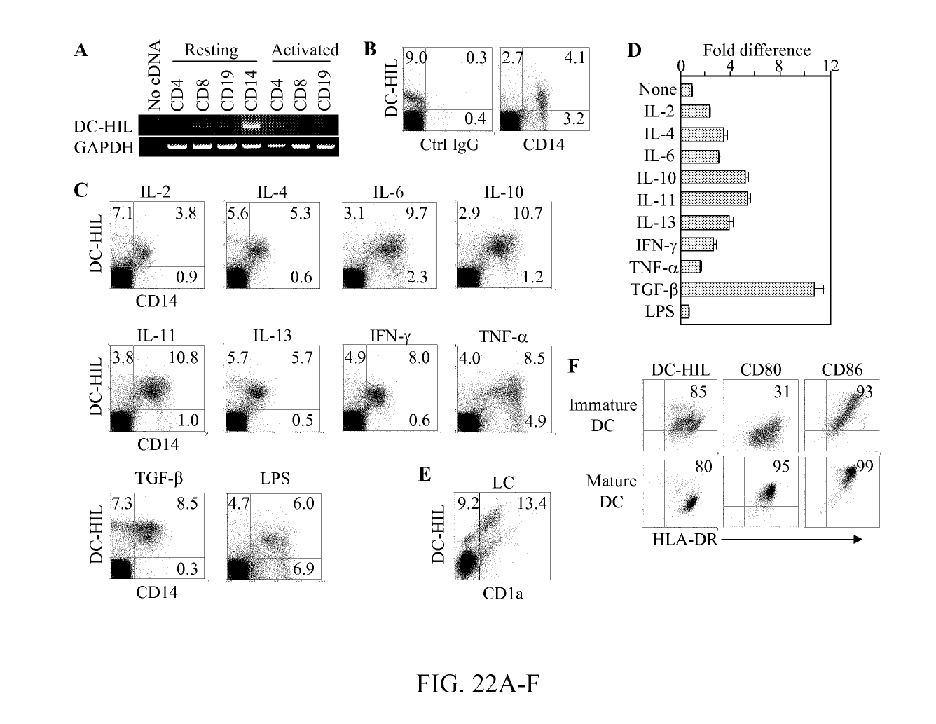

Among blood leukocytes, CD14+ monocytes display highest DC-HIL expression. Although a previous study showed high mRNA expression of DC-HIL by the human histiocytic lymphoma line U937 (Weterman et al., 1995), DC-HIL expression by normal leukocytes has not been examined. Human PBMC were sorted into CD4+ and CD8+ T cells, CD19+ B cells, and CD14+ monocytes. Total RNA from these cells was examined by RT-PCR for DC-HIL or GAPDH mRNA expression (FIG. 22A). DC-HIL mRNA was expressed highest by CD14+ cells. Actually, RT-PCR with RNA from CD14+ cells produced two PCR bands (FIG. 22A): a larger band (243 bp) corresponding to the full-length cDNA, and a smaller band (207 bp) that encoded a truncated isoform with a deletion of 12 amino acids in the proline-rich region (FIGS. 25A-D). This truncated form exhibits slightly higher potency with respect to binding activated T cells and inhibiting anti-CD3 Ab responses (unpublished data). A corresponding isoform has not been identified in mice.

By flow cytometry, 9% of PBMC stained with 3D5 anti-DC-HIL mAb, and 70% of these DC-HIL+ cells co-expressed the CD 14 marker (FIG. 22B). The inventors next assayed changes in surface expression following activation with 9 cytokines and LPS (FIGS. 22C and 22D). Two days after activation, CD14+ cells in PBMC were examined for surface expression of DC-HIL by flow cytometry. All 9 cytokines tested upregulated DC-HIL on CD14+ cells 2-to-10-fold and TGF-β was the strongest inducer. Indeed, TGF-P even induced DC-HIL-/CD14+ cells to express DC-HIL. By contrast, LPS had little to no effect on the expression.

DC-HIL is expressed preferentially by immature DC. To study correlation of DC-HIL expression levels with maturation status of APC, the inventors assayed the expression on immature vs. mature DC. Epidermal LC are known to represent the immature form of DC, and these cells were distinguished from other epidermal cells by expression of CD1a. In fact, Ficoll-enriched epidermal cell suspension contained 10% of CD1a+ LC, most of which expressed DC-HIL at very high levels (mean fluorescent intensity, MFI, of 524) (FIG. 22E). Note that DC-HIL+ CD1a− epidermal cells are melanocytes (unpublished data). The inventors next examined expression of DC-HIL on monocyte-derived DC. Immature DC were generated by culturing adherent PBMC with GM-CSF and IL-4 for 6 days, and induced to mature by culturing with a cytokine cocktail (IL-1β, TNF-α, and PGE2) for another 2 days. Phenotypic analysis showed the immature type to express lower levels of CD80 and CD86 (maturation markers) (FIG. 22F). Double-staining with anti-HLA-DR revealed the immature DC preparation to contain two subpopulations: HLA-DR_{high} and HLA-DR_{1ow} (FIG. 22F). Both expressed slightly higher levels of DC-HIL (MFI of 35.8) than mature DC (MFI of 24).

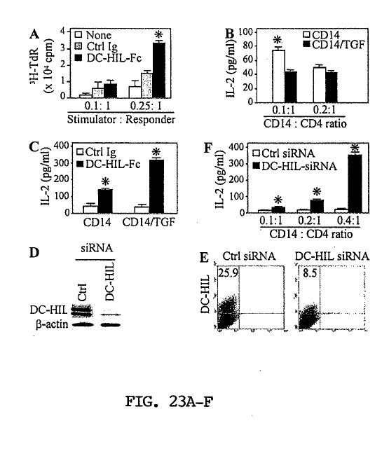

DC-HIL expression correlates inversely with allostimulatory capacity of CD14+ monocytes. The inventors next examined a role for DC-HIL in the mixed lymphocyte reaction (MLR), in which y-irradiated PBMC from one donor are mixed with purified CD4+ T cells from a different donor; T cell activation was assayed by 3H-thymidine incorporation. DC-HIL-Fc added to the MLR blocked the endogenous function of DC-HIL and augmented T cell proliferation 2-fold (FIG. 23A).

Because CD14+ monocytes possess APC capacity (Bhardwaj and Colston, 1988) and because TGF-β treatment amplified DC-HIL expression (FIG. 22D), the inventors next examined the effect of TGF-β-induced upregulation of DC-HIL expression on the APC function of CD14+ cells (FIG. 23B). CD14+ cells cultured for 2 days with or without TGF-β were mixed with allogeneic CD4+ T cells. At a CD14+ to CD4⁺ T cell ratio of 0.1:1, TGF-β-treated CD 14+ cells stimulated T cells to secrete about half of the IL-2 produced by T cells stimulated with untreated CD14+ cells. At a higher ratio (0.2:1), there was less to almost no difference. Under the higher ratio, the inventors examined the effect of DC-HIL-Fc on the MLR (FIG. 23C). Addition of soluble DC-HIL-Fc to CD14+ cells raised allostimulatory capacity to 3.1-fold higher than control cells, and its addition to TGF-β-treated CD14+ cells elevated such capacity even higher (7.7-fold). To more rigorously assess DC-HIL influence on APC capacity, the inventors examined effects of knocked-down DC-HIL expression on CD14+ cells. CD14+ cells were transfected with DC-HIL-targeted or control siRNA, and protein expression assayed by immunoblotting and flow cytometry (FIGS. 23D and E). DC-HIL siRNA markedly knocked-down protein expression in whole cell extracts (FIG. 23D) and surface expression on CD14+ cells (FIG. 23E). The inventors then assessed allostimulatory capacity of siRNA-transfected cells by titration of a constant number of CD4+ T cells with increasing numbers of transfected CD14+ cells (FIG. 23F). Compared to control cells, DC-HIL siRNA-transfected cells stimulated higher production of IL-2 by T cells at every dose point tested, up to 10-fold greater than control siRNA-CD14+ cells. Altogether, these results solidify the concepts that DC-HIL is a strong negative regulator of APC function and that TGF-β is a potent stimulator of DC-HIL expression.

### Example 9

### Discussion

These findings document the T cell inhibitory function of the DC-HIL/SD-4 pathway in humans. Human DC-HIL binds to SD-4 on activated (but not resting) T cells and this binding strongly blocks anti-CD3 responses of CD4+ and CD8+ T cells, including production of cytokines (IL-2, TNF-α, and IFN-γ) and entry into the S-phase. The inventors also showed that binding of DC-HIL to T cells transduces SD-4-dependent signaling. DC-HIL is expressed constitutively at high levels by CD14+ monocytes and immature DC (particularly epidermal LC) and this expression is regulated by TGF-β. The inventors also found DC-HIL to bind heparinase-sensitive structures on SD-4 (but not SD-1). Finally, SD-4 is a more potent inhibitor of the anti-CD3 Ab response than CTLA-4 and PD-1.

Whereas DC-HIL in mice is expressed highest by DC and macrophages and this expression is unaffected by treatment with cytokines or LPS (unpublished data), DC-HIL in human blood leukocytes is expressed highest by CD 14+ moncoytes and this expression is upregulated by various cytokines especially TGF-β. LPS, which is an activator of CD14+ cells, had almost no effect on DC-HIL expression, which is a feature in stark contrast with PD-L1, whose expression is increased markedly by the stimulus [22]. Note that TGF-β is the critical cytokine responsible for differentiation of CD14+ monocytes into epidermal LC (Geissmann et al., 1998), and the inventors have shown human LC to constitutively express DC-HIL at very high levels. Moreover, TGF-β can convert DC from immunostimulatory to tolerogenic phenotype, and it has been implicated as key to the ability of CD8+ regulatory T cells to suppress experimental autoimmune encephalomyelitis in mice (Rutella et al., 2006). Indeed, regulatory T cells produce TGF-β, which inhibits APC capacity of monocytes (Ersquerre et al., 2008), and malignant cells can secrete large amounts of TGF-β touted to play a role in suppressing anti-tumor immune responses (Teicher, 2007). Thus, the upregulated expression of DC-HIL on APC and the inhibitory effects of TGF-β may be interrelated.

Very recently, human CD4+ T cells were shown to express SD-2 and SD-4 upon activation, with both SD molecules capable of negatively regulating T cell activation induced by anti-CD3 Ab (Teixe et al., 2008). The inventors showed SD-4 expressed by activated T cells and anti-SD-4 Ab to inhibit the anti-CD3 Ab response of T cells. They were also able to show SD-1 on T cells, but not SD-2. These inconsistencies may be due to differences in PCR (real time vs. conventional PCR) and staining methods (intracellular vs. surface staining).

Unlike other inhibitory molecules (PD-1/PD-L1 and BTLA/HVEM) that bind their ligands via protein-protein interaction, DC-HIL appears to recognize SD-4 through non-peptide structures (heparin/heparan sulfate (HS) side chains), consistent with abrogation of its binding by heparinase treatment. Although SD-4 also bears chondroitin sulfate chains, these are not likely ligands for DC-HIL since chondroitin sulfate failed to block binding of DC-HIL to T cells (unpublished data). On the other hand, HS may not be the sole moiety responsible for DC-HIL/SD-4 binding since DC-HIL does not bind other HS-bearing proteins like SD-1, CD44, and glypicans. The inventors postulate DC-HIL to simultaneously recognize HS and a peptide epitope of SD-4. However, this possibility is confused by the observation that DC-HIL does not bind SD-4 engineered on Jurkat cells until after these cells are activated by Con A. Because different cells are known to express diverse HS structures corresponding to disparate binding activities (Esko and Selleck, 2002), the results suggest that DC-HIL recognizes a unique HS structure of SD-4 present on activated T cells, akin to interaction of selectins with ligands (PSGL-1) bearing T cell-specific glycosylation (Vestweber and Blanks, 1999). Regardless, there remains the question of why DC-HIL binds to SD-4 but not SD-1 on activated T cells especially since the inventors have no evidence of differences in HS chains on these molecules.

Autophosphorylation of serine and tyrosine residues on SD-4 following ligation by DC-HIL provides circumstantial evidence that intracellular signaling is responsible for DC-HIL/SD-4 inhibition of TCR-driven T cell activation. Because TCR signaling can be deleted by dephosphorylating molecules, and because CTLA-4, PD-1, and BTLA associate with protein tyrosine phosphatase (PTP) to mediate inhibitory function (Watanabe et al., 2003; Shlapatska et al., 2001; Chemnitz et al., 2004), the inventors postulate that SD-4 signaling involves some linkage with a PTP. However, unlike the other inhibitory molecules, SD-4 lacks an ITIM or other signaling motifs known to recruit the PTP-like SHP-1 and SHP-2 (Plas and Thomas, 1998). Absent direct linkage with PTP, our current investigation has focused on coupling of SD-4 function with a membrane-type PTP known to attenuate TCR signaling (Tangye et al., 1998).

Documenting the DC-HIL/SD-4 pathway to inhibit T cell activation in humans lays the foundation for future immunopharmacologic manipulation that may benefit patients with T cell-driven diseases like T cell lymphomas/leukemias, psoriasis, rheumatoid arthritis and inflammatory bowel disease.

### Example 10

### Results

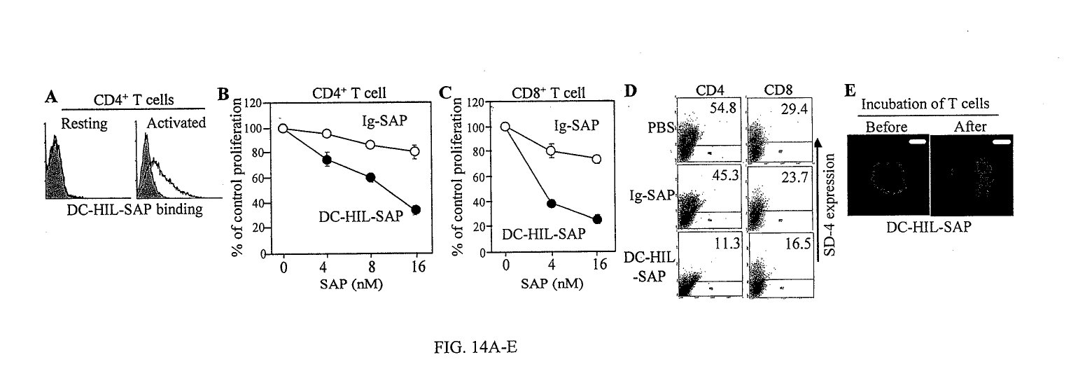

Toxin-conjugated DC-HIL selectively kills SD-4⁺ T-cells in vitro. The inventors chose saporin (SAP), an extensively-used and potent type I ribosome-inactivating protein as the toxin conjugated to DC-HIL. The inventors next verified that this immunotoxin retains the ability of DC-HIL to bind selectively to activated (but not resting) T-cells (FIG. 14A). Similar binding was noted for activated CD4⁺ and CD8⁺ T-cells (data not shown). The inventors then assessed the inhibitory effect of DC-HIL-SAP on T-cell activation (FIGS. 14B-C). CD4⁺ or CD8⁺ T-cells were activated by immobilized anti-CD3 Ab for 2 d and then incubated with DC-HIL-SAP (or control Ig-SAP) for 1 d. Next day, proliferation was measured by ³H-thymidine incorporation. DC-HIL-SAP blocked proliferation of CD4⁺ and CD8⁺ T-cells in a dose-dependent manner, with 70% reduction at the highest dose (16 nM saporin concentration). Reduced T-cell proliferation caused by DC-HIL-SAP was due to selective depletion of SD-4⁺ T-cells (FIG. 14D). The inventors next tracked internalization of DC-HIL-SAP into T-cells using a fluorescent marker (FIG. 14E). DC-HIL-SAP was allowed to bind activated T-cells on ice, then fluorescently labeled, incubated further at 37° C. for 30 min, and finally examined under confocal microscopy. Whereas all fluorescent labels were located on cell surfaces prior to experimentation, most labels clustered on the surface and then were internalized during incubation. Internalization would allow saporin to reach its target ribosomes where it can unleash its cytotoxic effect. These results indicate that toxin-conjugated DC-HIL selectively kills SD-4⁺ T-cells in vitro.

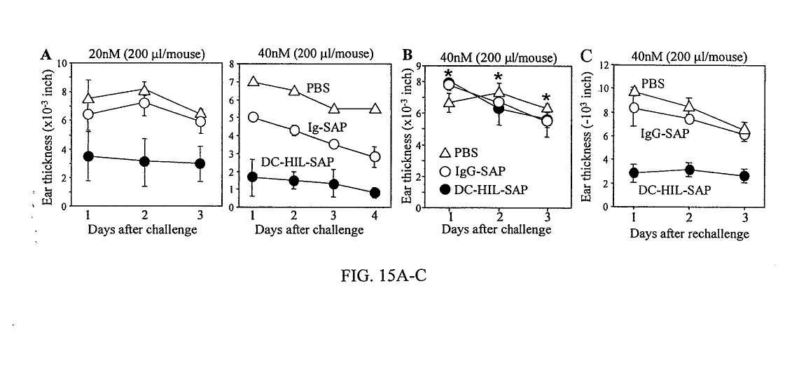

Infusion of DC-HIL-SAP blocks elicitation of CH. The inventors examined the effects of DC-HIL-SAP on CH to Ox (FIGS. 15A-B). BALB/c mice were sensitized with Ox on shaved abdominal skin (day 0) and elicited with Ox on ear skin (day 6); ear swelling was then measured daily. Mice were injected i.v. with DC-HIL-SAP (20 or 40 nM as saporin concentration), Ig-SAP, or PBS 3 h prior to challenge (FIG. 15A). Injection of the immunotoxin led to 60% and 80% reductions in ear swelling using 20 and 40 nM saporin, respectively. Ig-SAP also caused some diminution of ear swelling (10% and 30% respectively). Because the impact of DC-HIL is phase-dependent (affects elicitation but not sensitization), the inventors also examined effects of DC-HIL-SAP on the sensitization phase (FIG. 15B). As expected, neither DC-HIL-SAP nor control injected 3 hr before sensitization caused significant changes in ear swelling. To assess effects on memory, the same mice treated with DC-HIL-SAP at 40 nM (FIG. 15A) were kept for 2 wks and rechallenged by painting the ear with Ox. The unresponsiveness induced by DC-HIL-SAP persisted (validated in comparison to Ig-SAP-treated mice which showed robust ear swelling similar to PBS-controls) (FIG. 15C). These results indicate that a single infusion of DC-HIL-SAP during elicitation not only down-regulates the CH response but also produces an unresponsive state lasting at least 2 wks.

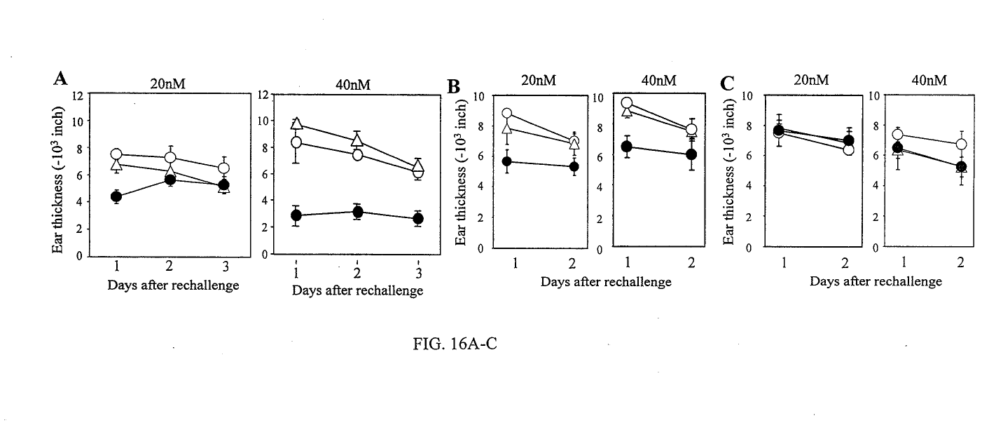

Antigen-specific unresponseviness induced by DC-HIL immunotoxin lasts until 3 weeks. To determine the duration of unresponsiveness to oxazolone in mice treated with DC-HIL-immunotoxin, BALB/c mice were sensitized and given intravenous injection of 20 or 40 mM of DC-HIL-SAP or Ig-SAP 3 h prior to challenge on day 6. Mice were kept for 1 week to completely recover to the baseline (no ear swelling) and then rechallenged with the same contact allergen oxazolone (the second challenge, FIG. 16A). No injection of SAP was done at the challenge. Mice injected with DC-HIL-SAP (40 nM) displayed unresponse to oxazolone (nearly 80% reduction in ear thickness). Injection of 20 nM DC-HIL-SAP showed 50% reduction. The inventors repeated this challenge 2 more times. At the third challenge, this unresponse was still noted in the mice treated with DC-HIL-Sap but not with PBS or ig-SAP control. (FIG. 16B). At the fourth challenge (3 weeks after injection), DC-HIL-SAP-treated mice developed ear swelling as strongly as control mice with PBS or Ig-SAP (FIG. 16C).

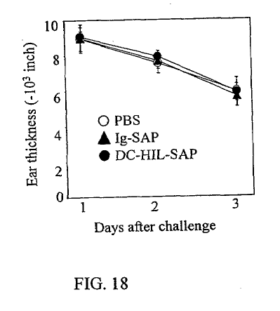

The inventors also examined whether this unresponse is achieved in an antigen-specific manner (FIG. 18). Mie were similarly treated with oxazolone and with injection of DC-HIL-SAP. One week after the first challenge, mice were then sensitized with a different contact allergen TNCB and mouse ears challenged with oxazolone (to left ears) or TNCB (to right ears). Left ears in mice with DC-HIL-SAP showed very little ear swelling, whereas their right ears showed ear swelling as strong as control mice. This result indicates that unresponsiveness induce by DC-HIL-SAP is antigen-specific.

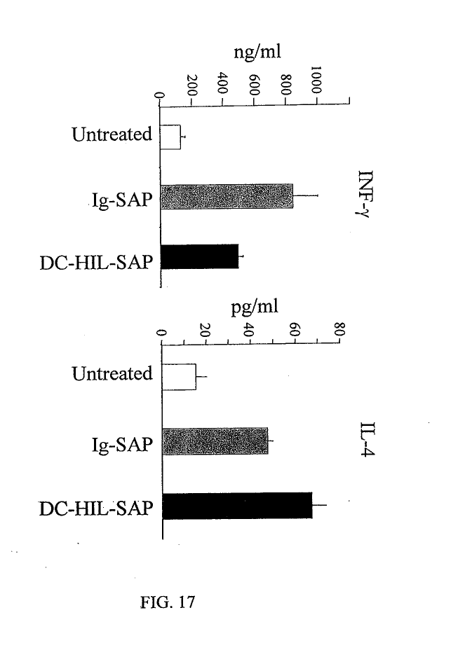

Shift from Th1 to Th2 response by DC-HIL-SAP. Acute T-cell-mediated inflammatory is caused by hyper Th2 response. In fact, mice immunized with oxazolone induce Th1-response that is denoted by high levels of IFNα production. To gain more insights into the mechanisms underlying blocking of CH elicitation by DC-HIL-SAP, the inventors analyzed balance of Th1 vs. Th2 response (indicated by IL-4 production) in lymph nodes of mice treated with DC-HIL-SAP. Lymph node cells were isolated from mice treated with DC-HIL-SAP or Ig-SAP (or untreated mice) and stimulated by anti-CD3 Ab. Production of IFN-g and IL-4 was measured in the culture supernatant (FIG. 17). DC-HIL-treated lymph node cells produced reduced levels of IFN-g (about 50% reduction), compared to Ig-SAP-treated mice. By contrast, IL-4 level was increased by mice with DC-HIL. This result shows that DC-HIL-SAP converted the Th1 type response to Th2-type, probably transiently. This fits the recent idea that Th2 response counteracts Th1 response.

### Example 11

### Materials and Methods

Cell isolation and culture. The human CTCL cell lines MJ (G11), Hut-78, and HH, obtained from American Type Culture Collection (ATCC, Rockville, Md.), were derived from peripheral blood of patients with MF, SS, and non-MF/SS aggressive CTCL, respectively (Gootenberg et al., 1981; Starkebaum et al., 1991; Popovic et al., 1983). Other T cell leukemia lines (Jurkat and Molt-4) were also obtained from ATCC. Samples of peripheral blood were obtained from 3 healthy donors, 6 SS patients, and MF with different malignancy stage (see Table 2). Blood samples were taken at two different time points from one SS patient. Samples were obtained during routine diagnostic assessments. The institutional review board of MD Anderson Cancer Center approved this study and the participants gave written informed consent. This study was conducted according to the Declaration of Helsinki Principles. CD4⁺ T cells were isolated from peripheral blood mononuclear cells (PBMCs) of healthy donors using CD4 T cell isolation kit (Myltenii, Biotec Auburn) according to the manufacturer's instructions. These normal T cells and the T cell lines were maintained in RPMI 1640 medium (Sigma Chemical Co., St Louis, Mo.) supplemented with 10% heat-inactivated fetal bovine serum (HyClone Laboratories, Logan, Utah).

TABLE 2
SD-4

| | Age/ | | | |
|---|---|---|---|---|
| Samples | Gender | Stages** | CD4+ 26− (%)^{#} | CD4+ SD4+ (%)^{#} |
| 1 | 61/M | SS IVA | 12.5 | 3.4 |
| 2 | 71/F | MF IV | 18.9 | 5.6 |
| 3* | 61/F | SS IVA | 28.7 | 5 |
| 4 | 70/F | SS IVB | 55.1 | 3.1 |
| 5 | 56/F | SS III | 75.3 | 3 |
| 6 | 39/F | SS IVB | 76.7 | 8.8 |
| 7* | 61/F | SS IVA | 87.1 | 13.6 |
| 8 | 75/F | SS | 93.15 | 2.9 |
| 9 | 58/F | SS IVB | 93.9 | 8.3 |
| 10 | 66/M | MF w/SS | 96 | 2.3 |
| 11 | 50/M | SS IVA | 96.6 | 9.2 |

*Samples #3 and #7 were from the same patient at different times.
**Malignancy stages.
^{#}Indicates positive cells among total PBMCs.

RT-PCR Analysis. Total RNA (1 μg) isolated from cell lines or normal CD4+ T cells was converted to the cDNA by reverse transcriptase (Life Technologies, Inc., Rockville, Md.) (Ariizumi et al., 1995). An aliquot (typically 5%) was used for PCR amplification (Ariizumi et al., 1995) using the primers: for CD160, 5′-GTTCACCATAAGCCAAGTCACACC-3′ (SEQ ID NO:20) and 5′-TTGCCCCAGCTTATATTTCCACAG-3′ (SEQ ID NO:21); for CTLA-4, 5′-GACCTGGCCCTGCACTCTCCT-3′ (SEQ ID NO:22) and 5′-AAAAACAACCCCGAACTAACTGCT-3′ (SEQ ID NO:23); for BTLA, 5′-ATGCCCTGTGAAATACTGTGCTAA-3′ (SEQ ID NO:24) and 5′-TGCCTGGTGCTTGCTTCTGT-3′ (SEQ ID NO:25); for PD-1, 5′-GGGCCCGGCGCAATGACA-3′ (SEQ ID NO:26) and 5′-GCGGGCGGGGGATGAGGT-3′ (SEQ ID NO:27); or for CD148, 5′-CTCCGCTCCAGCACCTTCTACAAC-3′ (SEQ ID NO:28) and 5′-GCACCGTCAGGGCTCTTCCAGTC-3′ (SEQ ID NO:29). Primers for SD-4 and GAPDH were the same as before (Chung et al., 2007a). Following 30-cycles of amplification, PCR products were separated electrophoretically on 1% agarose gel.

Western blot analysis. Whole cell extracts were prepared from T cell lines by lysis with 0.3% Triton X-100/DPBS for 15 min, followed by centrifugation for 20 min at 10,000×g (Sato et al., 2006b). An aliquot (40 μg) of extract was pretreated with heparinase I (0.1 U/ml) and III (0.2 U/ml) (Sigma) at 37° C. for 2 h before applying to SDS/4-20% gradient PAGE, followed by immunoblotting using anti-SD-4 Ab (μg/ml, eBiosciences). Color was developed by HRP-secondary Ab (1:10,000 dilution, Jackson ImmunoResearch, West Grove, Pa.) for 1 h and ECL plus system (Amersham Pharmacia Biotech, Piscataway, N.Y.).

Ab, Flow cytometry and confocal analysis. Abs used for fluorescent staining of cells include anti-SD-4 (5G9, H-140), anti-CD148 (143-41, H-300, CM, G15), anti-CD4 (RPA-T4), anti-CD3 (UGHT1), and anti-CD69 (FN50); all purchased from eBioscience (San Diego, Calif.). Three independent mAb directed against heparan sulfate (F58-10E4, HepSS-1, and F69-3G10) were obtained from Seikagaku Corporation (Japan). For analysis of surface expression, T cells or PBMCs (1×10⁵) were incubated with primary Ab (1-5 μg/ml) (or control IgG) and labeled fluorescently with 5 μg/ml PE- or FITC-conjugated secondary Ab, Jackson ImmunoResearch Laboratories Inc., West grove, PA). After extensively washing, cells were Fluorescence intensity of stained cells was analyzed by FACS Calibur (BD biosciences). For confocal analysis of CTCL lines, CTCL cells were stained with anti-SD-4 mAb (5 μg/ml) and FITC-anti-mouse IgG (5 μg/ml). After extensive washing, an aliquot was spotted on a slide glass, and fixed with 4% PFA at room temperature for 20 min. Fluorescently stained cells and skin tissues were examined using a Zeiss LSM510 laser scanning confocal microscope (Carl Zeiss Microimaging, Thornwood, N.Y.).

DC-HIL binding. CTCL lines or normal CD4⁺ T cells (day 3 after activation with 1 μg/ml immobilized anti-CD3 Ab) (1×10⁵ cells) were incubated with 10 μg/ml DC-HIL-Fc (fused with the Fc portion of mouse IgG) produced in transfected COS-1 cells as described previously (Chung et al., 2007). For experiments examining involvement of heparin, T cell lines (5×10⁵ cells) were pretreated with heparinase I (0.1 U/ml) and III (0.2 U/ml) (Sigma) at 37° C. for 2 h before binding assay. DC-HIL-Fc-treated cells were then fluorescently stained with PE-anti-mouse IgG (Fab) (5 μg/ml) followed by flowcytometry.

Cell cycle analysis. HH or HUT-78 cells were seeded onto a Petri dish (5×10⁵ cells/dish) and starved for 24 h in serum-free media to synchronize. After harvesting, cells were seed on microculture well (1×10⁵ cells/well) precoated with anti-CD3 Ab (increasing doses) plus DC-HIL-Fc or control Ig (each 10 μg/ml). After culturing for 18 h in the presence of 10% FCS-media, cells were subjected to cell cycle analysis using BrdU labeling and the BrdU flow kit (BD Biosciences), following the manufacturer's recommendations. BrdU incorporation (labeled with FITC) and DNA content (stained by 7-AAD) on a per-cell basis were analyzed by flow-cytometry.

T cell activation assay. HH or HUT-78 cells (without starvation) were cultured in microculture wells (1×10⁵ cells/well) precoated with anti-CD3 Ab (increasing doses) plus DC-HIL-Fc or control Ig (each 10 μg/ml) for 2 d. For HH line, cells were stained with anti-CD69 mAb and assayed by flow cytometry for frequency (%) of CD69+ cells in the culture. For HUT-78 line, culture supernatant was recovered and examined by ELIZA for production of IL-2 using IL-2 ELIZA kit (eBiosciences). To measure proliferation, cells were pulsed with ³H-thymidine (1 μCi/well) for the last 20 h of the culture period.

Anti-CTCL activity. DC-HIL-Fc or mouse IgG (as control) was biotinylated using EZ-link™ NHS-BIOTIN (Pierce, Rockford, Ill.) following manufacturer's recommendations. Normally, one Fc-protein molecule has 1-2 biotin molecules. Biotinilated protein was then conjugated with streptavidin-saporin (Advanced Targeting System, San Diego, Calif.) by mixing together at a molecular ratio (protein:saporin) of 1:1. Growing HH or HUT-78 cells were harvested, washed, seeded to microculture wells (5×10⁴ cells/well), and cultured with 10% FCS-media containing **100 U/ml of IL-2 (eBiosciences) and indicated concentration of saporin conjugates. Cells were pulsed with ³H-thymdine (1 μCi/well) for the last 20 h of the culture period (2 d).

Statistical analysis. Comparison of healthy donors and CTCL patients at frequency of SD-4 expression was made using Student's t test. The degree of linear relationship between two variables was evaluated using the Pearson correlation.

### Example 12

### Results

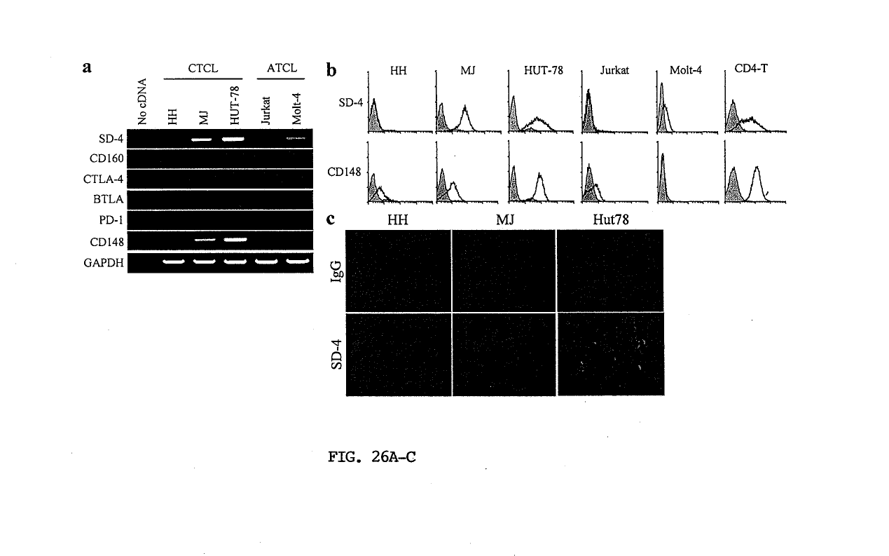

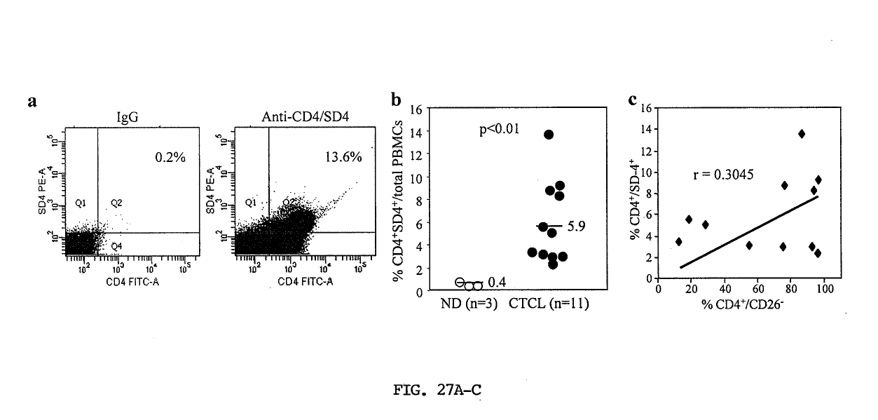

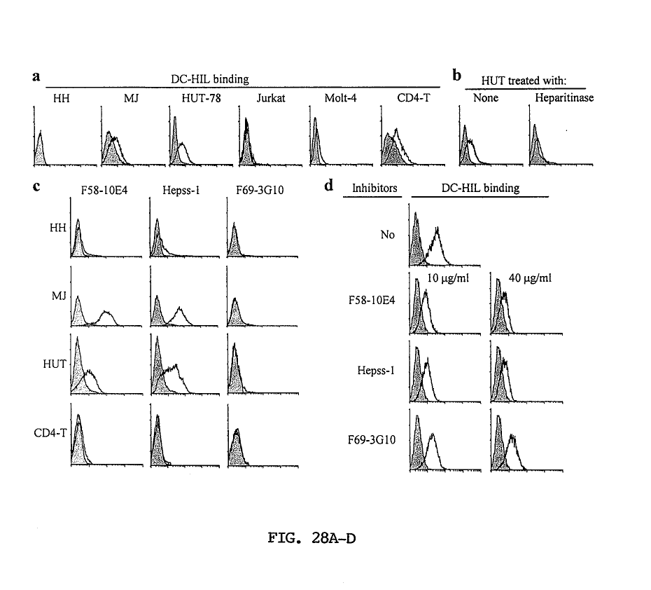

SD-4 is a co-inhibitory receptor expressed constitutively at high levels by CTCL. The inventors found previously that SD-4 is expressed primarily by effector/memory T cells in immunized mice. Because cutaneous T cell lymphoma (CTCL) cells also display effector/memory T cell phenotype, the inventors posited these cells to also express SD-4. The inventors examined mRNA expression of SD-4 in total RNA isolated from T cell lines including three CTCL lines (HH, MJ, and HUT-78) and two of acute T cell lymphoma (ATCL) lines (as control) (FIG. 26A). SD-4 mRNA was expressed constitutively at highest levels by HUT-78, at a lower level by MJ, and no expression by HH. By contrast, non-CTCL leukemia lines tested (Jurkat and Molt-4) expressed it no to very low levels. They then questioned whether other co-inhibitory receptors also are expressed by these CTCL cells. No mRNA for other receptors was expressed, which include CD160, CTLA-4, BTLA, and PD-1. Because their recent studies demonstrated that SD-4 employs tyrosine phosphatase activity of CD148 (manuscript in preparation), the inventors also examined mRNA expression of CD148 by CTCL cells. CD148 mRNA is co-expressed by MJ and HUT-78 cells, but not by SD-4-lacking cells tested. In consistent with mRNA expression levels, surface staining (flowcytometry) (FIG. 26B) and confocal analysis (FIG. 26C) revealed that MJ and HUT-78 cells express SD-4 and CD 148 on the cell, similarly to those by normal activated CD4⁺ T cells. Since MJ, Hut-78, and HH were derived from peripheral blood of patients with MF, SS, and non-MF/SS aggressive CTCL, respectively, our results suggest that MF and SS T cells express SD-4 but not other co-inhibitory receptors at the level of cell lines. Moreover, co-expression of CD148 suggests that SD-4 is functional in MJ and HUT-78 cells.

Peripheral blood CD4⁺ T cells of MF and SS patients express SD-4. The inventors next examined whether expression of SD-4 by CTCL lines is also true for CD4⁺ T cells from MF and SS patients. PBMCs isolated from 10 patients and 3 healthy donors (Table 2) were assayed by flowcytometry for frequency of CD4⁺ SD-4⁺ or of CD4⁺26⁻ cells (a marker for malignant T cells (Bemengo et al., 2001) (FIG. 27A). In healthy donors, frequency of CD4⁺ SD-4⁺ cells in PBMCs was 0.4% at the average, whereas this frequency was markedly increased to 5.9% in CTCL patients at a statistical significance of p<0.01 (FIG. 27B). The direct relationship between percentage values of CD4⁺CD26⁻ and CD4⁺ SD-4⁺ PBMCs was statistically significant to some extent (r=0.3045) (FIG. 27C). This relationship is further supported by flowcytometric data from one patient: Sample #3 and #7 were derived from the same patient but the different time point (Sample #3 was taken when CD4⁺CD26⁻ cells represented at 28.7%, and #7 at 93.15%). Sample #7 contained 13.6% CD4⁺ SD-4⁺ PBMCs, which was markedly elevated from 5% in Sample #3. These results indicate that CD4⁺ SD-4⁺ T cells in all MF and SS patients tested represent at a higher frequency than those in healthy normal donors. Thus, SD-4 expression level is likely correlate with the degree of peripheral blood involvement (assessed by CD4⁺ CD26⁻ percentage) in CTCL patients.

DC-HIL binds certain types of heparan sulfate expressed by CTCL cells. Since SD-4 is a receptor that binds to DC-HIL, the inventors examined binding of DC-HIL to the surface of CTCL cell lines and other T cells (FIG. 28A). As expected from expression levels of SD-4, DC-HIL bound to HUT-78 at highest levels and MJ at a lower level, and no binding to HH and other ATCL lines. Although normal activated CD4⁺ T cells expressed SD-4 at a level almost identical to that by these CTCL lines, they showed very low level of DC-HIL binding.

The inventors previous studies documented that DC-HIL binds to heparan sulfate-like molecules on SD-4. Thus, they addressed whether DC-HIL binding to CTCL cells is achieved in a similar manner. DC-HIL was pretreated with heparin or HUT-78 cells were pretreated with heparinase (a enzyme that remove heparan sulfate from the cell) prior to binding assay. Addition of heparin (data not shown) or heparinase treatment abrogated binding of DC-HIL to HUT-78 eclls (FIG. 28B). Taking advantage of 3 mAb that recognizes distinct structure of heparan sulfate, the inventors analyzed expression profile of these epitopes on CTCL cells (FIG. 28C). HUT-78 expressed 2 (F58-10E4 and Hepss-1) of 3 different epitope at highest levels, MJ at moderate levels, no to little expression by HH, which seems to correlate with the level of SD-4. Interestingly, no epitope expression was found on normal activated CD4⁺ T cells (even resting cells). The inventors then assayed inhibition of DC-HIL binding by these 3 anti-heparan sulfate mAb (FIG. 28D). HUT-78 cells were pretreated with/without each mAb before binding assay. In the absence of inhibitors, again DC-HIL bound to HUT-78 cells at high levels. This binding was greatly blocked by 10 or 40 mg/ml F58-10E4 or Hepss-1 mAb, but not with F69-3G10 mAb. These results indicate that CTCL cells bind DC-HIL at affinity much higher than normal CD4⁺ T cells through certain types of heparan sulfates.

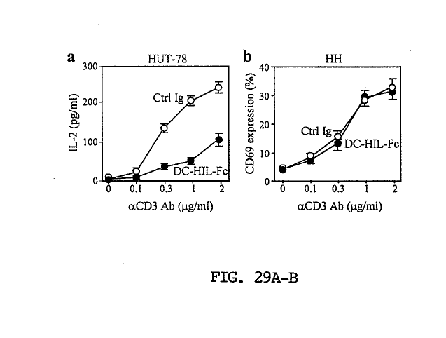

TCL-associated SD-4 responds to DC-HIL's inhibitory function by blocking secretion of IL-2 without blocking the entry to cell cycle. The inventors next examined whether SD-4 on CTCL cells is sensitive to DC-HIL's inhibitory function (FIGS. 29A-B). HUT-78 or HH (SD-4-control) cells were cultured in immobilized anti-CD3 Ab (increasing doses) plus DC-HIL-Fc or control Ig (a constant dose). Activation was measured by IL-2 production for HUT-78 cells or by % expression of CD69 for HH cells because the latter does not produce IL-2 upon activation. HUT-78 cells produced IL-2 triggered by anti-CD3 Ab in a dose-dependent manner (FIG. 29A). This production was inhibited by immobilized DC-HIl-Fc up to 70%. Expression of CD69 on HH cells was upregulated by anti-CD3 Ab dose-dependently, but it was not inhibited by co-application of DC-HIL-Fc. MJ cells were not available for the assay because they are devoid of CD3 expression. Thus, SD-4 on HUT-78 cells is functional similarly that on normal CD4⁺ T cells.

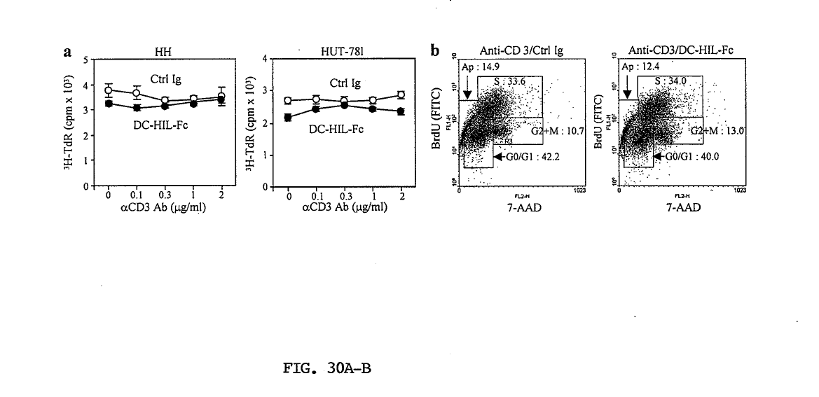

Since ligation of DC-HIL to SD-4 also inhibits proliferation of normal CD4⁺ T cells by blocking the entry to the cell cycle, the inventors assessed effect of DC-HIL binding on proliferation of HUT-78 or HH cells (FIGS. 30A-B). After starving, these cells were treated similarly in FIGS. 29A-B and proliferation was assayed by ³H-thymdine incorporation. Unlike normal CD4⁺ T cells, proliferative capacity of HUT-78 and even HH cells remained unchanged following DC-HIL treatment. This no effect on proliferation also was supported by cell cycle analysis: HT-78 cells were treated with anti-CD3 Ab plus DC-HIL and subjected to cell cycle analysis using FITC-anti-BrdU Ab (to detect proliferating cells) and 7-AAD (to measure DNA content). Depending on ratios of FITC vs. 7-AAD fluorescence, HUT-78 cells activated by anti-CD3 Ab were sorted into G0/G1 (42.2%), G2+M (10.7%), S (33.6%) and Ap (14.9%) (cells heating to apoptosis). DC-HIL treatment did not alter these frequencies. These results suggest that CTCL cells are deficient in a pathway for SD-4 to inhibit proliferation.

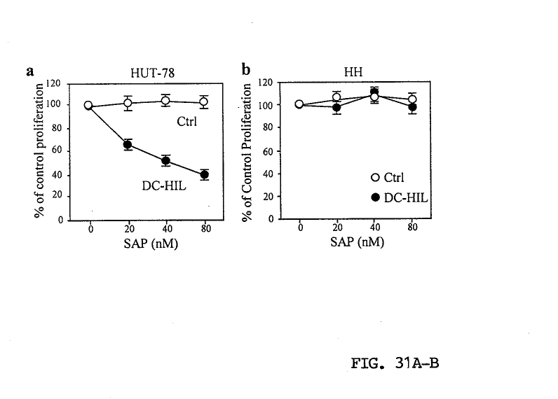

Toxin-bearing DC-HIL exhibits anti-CTCL activity. Having shown that DC-HIL binds to CTCL cells, the inventors posited that toxin-bearing DC-HIL kills CTCL cells. DC-HIL-Fc recombinant protein (or control Ig) was conjugated to the toxin saporin, an extensively-used and potent type I ribosome-inactivating protein (Flavell, 1998) and evaluated for efficacy to kill HUT-78 cells in vitro using ³H-thymidine incorporation assay (FIGS. 31A-B). Saporin-conjugated DC-HIL showed cytotoxic effect on proliferation of HUT-78 cells in a dose-dependent manner, with highest activity of 60% reduction at a highest concentration of 80 mM (FIG. 31A). Specificity for the reduction was supported by failure of control saporin to inhibit the proliferation and by no deleterious effect of DC-HIL-saporin on HH cells that lack SD-4 expression (FIG. 31B). These results raise the possibility that DC-HIL is a toxin carrier specifically delivering to CTCL cells.

### Example 13

### Discussion

CTCL-specific surface phenotypes can be identified, thus providing the ability to detect and monitor CTCL cells among circulating CD4⁺ T-lymphocytes (Nikolova et al., 2002). Such phenotypes include co-expression of the NK receptor (NKR) p140/KIR3DL2, SC5 inhibitory receptor, or CD26− by CD4⁺ T-lymphocytes, with the latter widely accepted to distinguish malignant from normal T cells. The inventors documented SD-4 to be expressed by some cells from MF and SS patients (but not from non-CTCL patients) at a level comparable to that found on normal CD4⁺ T cells activated by anti-CD3 Ab in vitro. Note little to no expression of SD-4 by normal resting CD4⁺ T cells. CD4⁺ PBMCs freshly isolated from 11 patients with MF or SS express it at markedly increased levels compared to those from normal donors. Importantly, such expression correlates positively and significantly with the degree of peripheral blood malignancy in MF and SS, as assessed by percentage of CD4⁺ CD26⁻ PBMCs. Thus, SD-4 expression by CTCL cells should augment precision in identifying susceptible CTCL cells and may contribute to new development of diagnosis for the peripheral blood malignancy in future.

There are a variety of therapeutic modalities available for CTCL patients, one of which is a newly developed Denileukin diftitox (or Ontak), a IL-2 recombinant protein fused with diphtheria toxin. This binds to the high affinity IL-2 receptor (or high CD25) and delivers the toxin into target cells, thus killing them. Recent studies have documented that high CD25 expression is associated with advanced CTCL and that in a phase III study, overall, 30% of the 71 patients with CTCL treated with denileukin diftitox had a response (20% partial responses rate; 10% complete response rate) (Zhang et al., 2008; Horwitz, 2008). Response rate and progression-free survival were superior for patients treated with denileukin diftotox compared with patients receiving placebo. However, there were frequent adverse events including constitutional and gastrointestinal symptoms. A conceptual disadvantage of denileukin diftotox is depletion of all activated T cells including those in the memory pool that are important for protective immunity. Like IL-2 protein, DC-HIL binds to activated effector/memory T cells in PBMCs of normal donors through the ligand, particular types of heparan sulfates on SD-4 expressed by T cells but not B cells nor by other leukocytes (Chung et al., 2007). Unlike IL-2 receptor, SD-4 is expressed by only 25% of activated CD25⁺ T cells. The inventors' finding that toxin-conjugated DC-HIL exhibits efficient anti-CTCL activity provides the rationale for its therapeutic use in CTCL.

SD-4 belongs to the syndecan family of transmembrane receptors heavily laden with heparin/heparan sulfate (HS) chains consisting of alternating disaccharide residues (glucuronic acid or iduronic acid with glucosamine) (Baciu et al., 2000; Charnaux et al., et al., 2005; Ishiguro et al., 2003). The inventors showed that DC-HIL ligands are likely particular types of HS detected by two different mAb clones (F58-10E4 and Hepss-1) that block DC-HIL binding. Limited information for structures of these mAb epitopes does not allow us to identify the exact structures. During studies about HS, the inventors found a distinct difference in the HS expression between normal and CTCL cells. CTCL cells express SD-4 as high as activated CD4⁺ T cells of normal donors, whereas the former express these two epitopes at high levels but at no to very low levels by normal cells, consistent with the extent of DC-HIL binding. This aberrant (or abnormally increased) glycosylation is a likely carbohydrate marker to discriminate CTCL cells from normal cells (Ohyama, 2008; Ono and Hakomori, 2004).

In normal T cells, ligation of DC-HIL to SD-4 inhibits production of IL-2 triggered by anti-CD3 Ab, accompanied with blocking of proliferation and the entry of T cells to the cell cycle. In CTCL cells, SD-4 is capable of inhibiting the IL-2 production but incapable of blocking proliferation of HUT-78 cells and the entry to the cell cycle. However, failure of blocking proliferation may be due to very low responsiveness to anti-CD3 Ab stimulation. Since CTCL cells have variability in phenotype and function, other CTCL lines may respond to DC-HIL's inhibitory signal to proliferation. Although the inventors showed that CTCL cells express SD-4 but not other known co-inhibitory receptors (PD-1, BTLA, CD160, and CTLA-4), it does not mean that it is only one inhibitory mechanism for TCR-driven activation. A previous report shows that a newly identified SC5 receptor also is expressed by CTCL cells and that it inhibits anti-CD3 Ab response of a CTCL line (inhibition of IL-2 production and proliferation) (Nikolova et al., 2001; Nikolova et al., 2002). It may be likely that CTCL cells retain several inhibitory mechanisms that normally limit activation of normal T cells. Then, there is a critical question of whether retaining of such feedback mechanisms contributes to uncontrolled growth of CTCL cells. Since SD-4 has functions other than adhesion and T cell inhibition (Couchman and Woods, 1999), the inventors speculate that another function of SD-4 may promote oncogenesis of CTCL cells. Thus, SD-4 may be a new surface marker to distinguish some CTCL lymphocytes. Moreover, as an inhibitor of the anti-CD3 Ab response it is a potentially useful target for treatment of CTCL.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,680,338
U.S. Pat. No. 4,843,092
U.S. Pat. No. 5,141,648
U.S. Pat. No. 5,440,013
U.S. Pat. No. 5,446,128
U.S. Pat. No. 5,475,085
U.S. Pat. No. 5,563,250
U.S. Pat. No. 5,618,914
U.S. Pat. No. 5,670,155
U.S. Pat. No. 5,672,681
U.S. Pat. No. 5,674,976
U.S. Pat. No. 5,710,245
U.S. Pat. No. 5,840,833
U.S. Pat. No. 5,856,456
U.S. Pat. No. 5,859,184
U.S. Pat. No. 5,880,270
U.S. Pat. No. 5,929,237
Acuto and Michel, Nat. Rev. Immunol., 3:939-951, 2003.
Anderson et al., Nat. Genet., 30:81-85, 2002.
Ariizumi et al., J. Immunol., 154:6031-6039, 1995.
Baciu et al., J Cell Sci., 113(Pt 2):315-324, 2000.
Bernengo et al., Br. J. Dermatol., 144:125-135, 2001.
Bhardwaj and Colston, Eur. J. Immunol. 18:691-696, 1988.
Brown et al., J. Immunol., 170:1257-1266, 2003.
Bycroft et al., EMBO J, 18:297-305, 1999.
Cardin and Weintraub, Arteriosclerosis, 9:21-32, 1989.
Carreno and Collins, Annu. Rev. Immunol., 20:29-53, 2002.
Chambers and Allison, Curr. Opin. Immunol., 9:396-404, 1997.
Charnaux et al., FEBSJ, 272:1937-1951, 2005.
Chemnitz et al., J. Immunol., 173:945-954, 2004.
Chung et al., Blood, 109(10):4320-4327, 2007a.
Chung et al., J. Immunol., 179(9):5778-5784, 2007b.
Couchman and Woods, J Cell Sci., 112(Pt 20):3415-3420, 1999.
Cruz and Bergstresser, Photodermatol., 5:151-161, 1988.
Cruz et al., Photodermatol., 5:126-132, 1988.
Demotz et al., Eur. J. Immunol., 23:425-432, 1993.
Dong and Chen, Mol. Med., 81:281-287, 2003.
Dong et al., Nat. Med., 5:1365-1369, 1999.
Esko and Selleck, Annu. Rev. Biochem., 71:435-471, 2002.
Esquerre et al., Proc. Natl. Acad Sci. USA, 105:2550-2555, 2008.
Flavell, Curr. Top Microbiol. Immunol., 234:57-61, 1998.
Freeman et al., J. Exp. Med., 192:1027-1034, 2000.
Geissmann et al., J. Exp. Med., 187:961-966, 1998.
Gibot et al., J. Exp. Med., 200:1419-1426, 2004.
Gootenberg et al., J. Exp. Med., 154:1403-1418, 1981.
Gorczynski et al., J. Immunol., 172:7744-7749, 2004.
Hahn and Tsao, In: Dubois' Lupus Erythematosus, 4^{th} Ed, Wallace and Hahn (Eds.), Lea and Febiger, Philadelphia, 195-201, 1993.
Horowitz and Simons, J. Biol. Chem., 273:25548-25551, 1998.
Horwitz, Clin. Lymphoma Myeloma, 8(Suppl 5):S187-S192, 2008.
Hsieh et al., Science, 260:547-549, 1993.
Inaba et al., J. Exp. Med., 176:1693-1702, 1992.
Ishiguro et al., Glycoconjugate J, 19:315-318, 2003.
Johannesson et al. J. Med. Chem., 42(22):4524-4537, 1999.
Johnson et al., In: Biotechnology And Pharmacy, Pezzuto et al. (Eds.), Chapman and Hall, NY, 1993.
Kay et al., FASEB J., 14:231-241, 2000.
Kotzin and O'Dell, In: Samler's Immunologic Diseases, 5^{th} Ed., Frank et al. (Eds.), Little Brown & Co., Boston, 667-697, 1995.
Kotzin, Cell, 85:303-306, 1996.
Krummel and Allison, J. Exp. Med., 182:459-465, 1995.
Latchmann et al., Nat. Immunol., 2:261-268, 2001.
Liang and Sha, Curr. Opin. Immunol., 14:384-390, 2002.
Linsley and Ledbetter, Annu. Rev. Immunol., 11:191-212, 1993.
Mantle et al., Adverse Drug React. Toxicol. Rev., 20(2):89-103, 2001.
Monney et al., Nature, 415:536-541, 2002.
Nikolova et al., J. Invest. Dermatol., 116:731-738, 2001.
Nikolova et al., Leuk. Lymphoma, 43:741-746, 2002.
Nishimura et al., Immunity, 11:141-151, 1999.
Nishimura et al., Int. Immunol., 10:1563-1572, 1998.
Ohnishi et al., Int. Immunol., 6:817-830, 1994.
Ohnishi et al., Nephrol. Dial. Transplant., 9:1747-1750, 1994.
Ohyama, Int. J. Clin. Oncol., 13:308-313, 2008.
Okazaki et al., Curr. Opin. Immunol., 14:779-782, 2002.
Ono and Hakomori, Glycoconj. J, 20:71-78, 2004.
Plas and Thomas, J. Mol. Med., 76:589-595, 1998.
Ponting et al., Curr. Biol., 9:R585-R588, 1999.
Popovic et al., Science, 219:856-859, 1983.
Remington's Pharmaceutical Sciences, 18th Ed. Mack Printing Company, 1289-1329, 1990.
Ruoslahti, Annu. Rev. Cell Dev. Biol., 12:697-715, 1996.
Rutella et al., Blood, 108:1435-1440, 2006.
Sabatos et al., Nat. Immunol., 4:1102-1110, 2003.
Safadi et al., J Cell Biol., 84:12-26, 2001.
Sanchez-Fueyo et al., Nat. Immunol., 4:1093-1101, 2003.
Sato et al., J. Biol. Chem., 281:38854-38866, 2006a.
Sato et al., Mol. Immunol., 43:1443-1453, 2006b.
Sedy et al., Nat. Immunol., 6:90-98, 2005.
Shi et al., Blood, 108:2678-2686, 2006.
Shikano et al., J. Biol. Chem., 276:8125-8134, 2001.
Shlapatska et al., J. Immunol., 166:5480-5487, 2001.
Starkebaum et al., Int. J Cancer, 49:246-253, 1991.
Subudhi et al., J. Clin. Invest., 113:694-700, 2004.
Takao et al., Photodermatol. Photoimmunol. Photomed., 18: 5-13, 2002.
Tangye et al., J. Immunol., 161:3249-3255, 1998.
Teicher, Clin. Cancer Res., 13:6247-6251, 2007.
Teixe et al., Mol. Immunol., 45:2905-2919, 2008.
Tivol et al., Immunity, 3:541-547, 1995.
Tseng et al., J. Exp. Med., 193:839-846, 2001.
Tsushima et al., Eur. J. Immunol., 33:2773-2782, 2003.
Vestweber and Blanks, Physiological Reviews, 79:181-213, 1999.
Vita et al., Biopolymers, 47:93-100, 1998.
Walunas et al., Immunity, 1:405-413, 1994.
Watanabe et al., Nat. Immunol., 4:670-679, 2003.
Wawrzynczak and Thorpe, FEBS Lett., 207(2):213-216, 1986.
Wegrowski et al., Clin. Exp. Immunol., 144:485-493, 2006.
Weisshoff et al., Eur. J. Biochem., 259(3):776-788, 1999.
Weterman et al., Int. J Cancer, 60:73-81, 1995.
Wilson et al., Biochem. J., 275:529-534, 1991.
Woods and Couchman, Opin. Cell Biol., 13:578-583, 2001.
Zhang et al., J. Invest. Dermatol, 128:2728-2735, 2008.

What is claimed:

1. A method of reducing T-cell induced inflammation in a subject comprising administering to said subject a conjugate comprising (a) DC-HIL or a syndecan-4-binding fragment thereof, and (b) a toxin.

2. The method of claim 1, wherein said T-cell induced inflammation is host-versus-graft disease, psoriasis, atopic dermatitis, contact hypersensitivity, autoimmune disease, or skin graft rejection.

3. The method of claim 1, wherein said subject is a human, a mouse, a rat, a dog or a cat.

4. The method of claim 1, wherein said conjugate comprises DC-HIL.

5. The method of claim 1, wherein said conjugate comprises a syndecan-4-binding fragment of DC-HIL comprising the DC-HIL Ig-like domain.

6. The method of claim 1, wherein said toxin is saporin, ricin, botulinum toxin or diptheria toxin.

7. The method of claim 1, wherein administration comprises intravenous, intra-arterial, topical, intralesional, subcutaneous, intraperitoneal, intradermal, or intransal administration.

8. The method of claim 1, further comprising administering to said subject a second anti-inflammatory treatment.

9. The method of claim 8, wherein said second anti-inflammatory treatment comprises an immunosuppressant, a steroid or an NSAID.

10. The method of claim 1, further comprising a second administration of said conjugate.

11. A method of inhibiting a syndecan-4-positive T-lymphoma or T-leukemia cell in a subject comprising administering to said subject a conjugate comprising (a) DC-HIL or a syndecan-4-binding fragment thereof, and (b) a toxin.

12. The method of claim 11, wherein inhibiting comprises reducing the viability or proliferation of said cell.

13. The method of claim 11, wherein said subject is a human, a mouse, a rat, a dog or a cat.

14. The method of claim 11, wherein said conjugate comprises DC-HIL.

15. The method of claim 11, wherein said conjugate comprises a syndecan-4-binding fragment of DC-HIL comprising the DC-HIL Ig-like domain.

16. The method of claim 11, wherein said toxin is saporin, ricin, botulinum toxin or diptheria toxin.

17. The method of claim 11, wherein administration comprises intravenous, intra-arterial, intra-lymphatic, intralesional, subcutaneous, intraperitoneal, intradermal or intranasal administration.

18. The method of claim 11, further comprising administering to said subject a second anti-lymphoma or -leukemia treatment.

19. The method of claim 18, wherein said second anti-lymphoma or -leukemia treatment comprises chemotherapy, radiotherapy, IFNα and/or anti-CD20 antibody.

20. The method of claim 11, further comprising a second administration of said conjugate.

21. A conjugate comprising (a) DC-HIL or a syndecan-4-binding fragment thereof; and (b) a toxin.

22. The conjugate of claim 21, wherein said conjugate comprises DC-HIL.

23. The conjugate of claim 21, wherein said conjugate comprises a syndecan-4-binding fragment of DC-HIL comprising the DC-HIL Ig-like domain.

24. The conjugate of claim 21, wherein said toxin is saporin, ricin, or diptheria toxin.

25. The conjugate of claim 21, wherein said conjugate is disposed in a pharmaceutically acceptable buffer, carrier or diluent.
